(19) United States
(12) Patent Application Publication
(10) Pub. No.: US 2013/0331630 A1
(43) Pub. Date: Dec. 12, 2013

(54) METHODS FOR SUPPRESSING DEHYDROGENATION

(71) Applicant: ELEVANCE RENEWABLE SCIENCES, INC., Woodridge, IL (US)

(72) Inventors: Bradon J. Dreyer, Woodridge, IL (US); Brian M. Pease, Aurora, IL (US)

(21) Appl. No.: 13/911,504

(22) Filed: Jun. 6, 2013

Related U.S. Application Data
(60) Provisional application No. 61/658,592, filed on Jun. 12, 2012.
(60) Provisional application No. 61/658,658, filed on Jun. 12, 2012.
(60) Provisional application No. 61/658,730, filed on Jun. 12, 2012.
(60) Provisional application No. 61/658,778, filed on Jun. 12, 2012.

(51) Int. Cl. C07C 6/04

(52) U.S. Cl. CPC C07C 6/04
USPC 585/645; 585/643

(57) ABSTRACT

A method for suppressing dehydrogenation includes reacting an optionally functionalized olefin reactant in a metathesis reaction to form an olefin metathesis product; and providing a dehydrogenation suppression agent in admixture with (a) the olefin metathesis product and/or the optionally functionalized olefin reactant, and (b) a metal-containing material selected from the group consisting of residual metathesis catalyst, a hydrogen transfer agent, and a combination thereof, under conditions that are sufficient to passivate at least a portion of the metal-containing material. Methods of refining natural oils and methods of producing fuel compositions are also described.

### CROSS REFERENCE TO RELATED APPLICATIONS

This application claims the benefit of U.S. Provisional Patent Application No. 61/658,592, filed Jun. 12, 2012, U.S. Provisional Patent Application No. 61/658,658, filed Jun. 12, 2012, U.S. Provisional Patent Application No. 61/658,730, filed Jun. 12, 2012, and U.S. Provisional Patent Application No. 61/658,778, filed Jun. 12, 2012, each of which is incorporated herein by reference.

### FEDERALLY SPONSORED RESEARCH OR DEVELOPMENT

This invention was made with government support under Contract No. DE-EE0002872 awarded by Department of Energy. The government has certain rights in the invention.

### BACKGROUND

The olefin metathesis reaction is a highly versatile and powerful technique for the synthesis of alkenes. Transition metal carbene complexes—particularly those incorporating ruthenium, molybdenum, or tungsten—are popular catalysts for metathesis. However, the yield of certain desired metathesis products can be significantly reduced by double bond isomerization. Such isomerization typically results from a metal-containing material (e.g., residual metathesis catalyst and/or its byproducts) being present in the reaction mixture. The problem becomes particularly acute if the metathesis mixture is heated and/or distilled in the presence of the metal-containing material.

In addition to being susceptible to undesirable olefin isomerization, some metathesis products—particularly though not exclusively methylene-interrupted polyolefin metathesis products—are susceptible to dehydrogenation (which can occur in combination with or separately from olefin isomerization). As with olefin isomerization, dehydrogenation typically results from a metal-containing material (e.g., a material that facilitates hydrogen transfer) being present in the reaction mixture. Moreover, the dehydrogenation of certain metathesis products can lead to the formation of volatile organic compounds (VOCs), including but not limited to benzene—a highly undesirable and carcinogenic byproduct.

The problem of unwanted dehydrogenation is particularly acute in metathesis reactions that involve polyunsaturated fatty acids and fatty acid derivatives (e.g., monoglycerides, diglycerides, triglycerides, etc.). When subjected to a metathesis reaction, these polyunsaturated fatty acids and their derivatives can generate a mixture of linear hydrocarbon olefins, olefinic fatty acid esters, and unsaturated cyclic byproducts. In the presence of a metal-containing material (e.g., such as a residual metathesis catalyst and/or its byproducts, a hydrogenation catalyst, and/or the like), the olefin metathesis products can be dehydrogenated (with or without a prior and/or subsequent isomerization). Moreover, some unsaturated cyclic byproducts—such as cyclohexadiene (CHD)—can be converted into benzene upon dehydrogenation, thereby contaminating a desired metathesis product with an IARC Group 1 carcinogen. Thus, although metathesis reactions involving natural oil feedstocks (e.g., vegetable and seed-based oils) are presently of considerable interest in the manufacture of biofuels, waxes, plastics, and the like, the problem of carcinogenic benzene formation (and/or other VOCs produced via unwanted dehydrogenations) must be addressed in order extend the feasibility of the approach.

A dehydrogenation suppression agent capable of passivating metal-containing materials, such as residual metathesis catalyst and/or hydrogen transfer agents present in admixture with olefinic metathesis product, is needed.

### SUMMARY

Methods are disclosed for suppressing dehydrogenation. In one embodiment, the method comprises reacting an optionally functionalized olefin reactant in a metathesis reaction to form an olefin metathesis product. The method further comprises providing a dehydrogenation suppression agent in admixture with (a) the olefin metathesis product and/or the optionally functionalized olefin reactant, and (b) a metal-containing material selected from the group consisting of residual metathesis catalyst, a hydrogen transfer agent, and a combination thereof, under conditions that are sufficient to passivate at least a portion of the metal-containing material. Non-passivated metal-containing material is configured to participate in, catalyze, promote, and/or facilitate dehydrogenation of the optionally functionalized olefin reactant and/or the olefin metathesis product.

In certain embodiments, the dehydrogenation suppression agent comprises phosphorous. In other embodiments, the dehydrogenation suppression agent comprises nitrogen. In yet other embodiments, the dehydrogenation suppression agent comprises a quinone, hydroquinone, or a combination thereof.

### BRIEF DESCRIPTION OF THE DRAWINGS

FIG. 1 shows one possible mechanistic pathway by which 1,4-CHD can form in a metathesis reaction involving an 18:3 fatty acid and/or a derivative thereof.

FIG. 2 shows one possible mechanistic pathway by which 1,4-CHD can form in a metathesis reaction involving an 18:2 fatty acid and/or a derivative thereof.

FIG. 3 shows representative phosphorous acid derivatives for use in accordance with the present teachings.

FIG. 4 shows representative phosphinic acid derivatives for use in accordance with the present teachings.

FIG. 5 shows a process flow diagram depicting a representative scheme for dehydrogenation suppression, and shows an optional extraction, separation, and transesterification.

FIG. 6 is a schematic diagram of one embodiment of a process to produce a fuel composition and a transesterified product from a natural oil.

FIG. 7 is a schematic diagram of a second embodiment of a process to produce a fuel composition and a transesterified product from a natural oil.

### DETAILED DESCRIPTION

An effective methodology for suppressing the dehydrogenation of olefin metathesis products and/or reactants has been discovered and is described herein. As further described below, the inventive methodology involves adding a dehydrogenation suppression agent to a mixture of (i) olefin metathesis product and/or reactant, and (ii) metal-containing material (e.g., residual metathesis catalyst, hydrogen transfer agent, and/or the like). In some embodiments, the dehydrogenation suppression agent includes phosphorous. In some embodiments, the dehydrogenation suppression agent includes nitrogen. In some embodiments, the dehydrogenation suppression agent includes a quinone, a hydroquinone, or a combination thereof. In some embodiments, the dehydrogenation suppression agent—in addition to or as an alternative to suppressing dehydrogenation—further acts as an isomerization suppression agent, thereby facilitating preservation of the original location of a carbon-carbon double bond created during a metathesis reaction.

### DEFINITIONS

Throughout this description and in the appended claims, the following definitions are to be understood:

The term “olefin” refers to a hydrocarbon compound containing at least one carbon-carbon double bond. As used herein, the term “olefin” encompasses hydrocarbons having more than one carbon-carbon double bond (e.g., di-olefins, tri-olefins, etc.). In some embodiments, the term “olefin” refers to a group of carbon-carbon double bond-containing compounds with different chain lengths. In some embodiments, the term “olefin” refers to poly-olefins, straight, branched, and/or cyclic olefins.

The term “suppressing” as used in reference to the dehydrogenation of an olefin refers to an inhibitory effect on the olefin's susceptibility towards dehydrogenation under a given set of conditions. Similarly, the term “suppressing” as used in reference to the isomerization of an olefin refers to an inhibitory effect on the olefin's susceptibility towards isomerization under a given set of conditions. It is to be understood that the term “suppressing” encompasses but does not necessarily imply 100% suppression (i.e., 0% dehydrogenation and/or isomerization).

The term “dehydrogenation” refers to an elimination of hydrogen from a molecule that results in the formation of a carbon-carbon double bond.

The term “isomerization” refers to the migration of a carbon-carbon double bond from one location in a molecule to another location within the molecule (e.g., from a terminal position to an internal position and/or from an internal position to a terminal position and/or from a first internal position to a second internal position and/or from a first terminal position to a second terminal position, etc.). As used herein, the term “isomerization” includes both single migrations from an initial position to a final position as well as successive migrations from an initial position through one or a plurality of intermediate positions to a final position.

The phrase “olefin metathesis product” refers to any product produced in a metathesis reaction that contains at least one carbon-carbon double bond. In some embodiments, the “olefin metathesis product” can refer to a major product and/or one or a plurality of minor products formed in the metathesis reaction. In some embodiments, the “olefin metathesis product” refers to one or a plurality of minor by-products. In some embodiments, the “olefin metathesis product” is formed directly from starting reagents via a single metathesis reaction. In some embodiments, the “olefin metathesis product” is formed via a plurality of metathesis reactions (e.g., through an intermediate metathesis product that, under the conditions of the reaction, undergoes further metathesis to yield the “olefin metathesis product”). In some embodiments, the “olefin metathesis product” is cyclic. In some embodiments, the “olefin metathesis product” is an unfunctionalized hydrocarbon compound. In some embodiments, the phrase “olefin metathesis product” subsumes the term “olefin.” In some embodiments, the “olefin metathesis product” is functionalized and contains one or a plurality of additional functional groups in addition to its at least one carbon-carbon double bond.

The term “functionalized” and the phrase “functional group” refer to the presence in a molecule of one or more heteroatoms at a terminal and/or an internal position, wherein the one or more heteroatoms is an atom other than carbon and hydrogen. In some embodiments, the heteroatom constitutes one atom of a polyatomic functional group. Representative functional groups including but are not limited to halides, alcohols, amines, carboxylic acids, carboxylic esters, ketones, aldehydes, anhydrides, ether groups, cyano groups, nitro groups, sulfur-containing groups, phosphorous-containing groups, amides, imides, N-containing heterocycles, aromatic N-containing heterocycles, salts thereof, and the like, and combinations thereof.

The phrase “metathesis reaction” refers to a chemical reaction involving a single type of olefin or a plurality of different types of olefin, which is conducted in the presence of a metathesis catalyst, and which results in the formation of at least one new olefin product. The phrase “metathesis reaction” encompasses self-metathesis, cross-metathesis (aka co-metathesis; CM), ring-opening metathesis (ROM), ring-opening metathesis polymerizations (ROMP), ring-closing metathesis (RCM), acyclic diene metathesis (ADMET), and the like, and combinations thereof. In some embodiments, the phrase “metathesis reaction” refers to a chemical reaction involving a natural oil feedstock.

The phrase “phosphorous oxo acid” refers to a molecule that comprises a P—OH moiety in which the hydrogen atom is ionizable.

The phrase “higher acid” as used in reference to a phosphorous oxo acid refers to an acid in which phosphorous is in an oxidation state of +5.

The phrase “lower acid” as used in reference to a phosphorous oxo acid refers to an acid in which phosphorous is in an oxidation state below +5 (e.g., P^{III}).

The phrase “ester of a phosphorous oxo acid” refers to a molecule that comprises a P—OR bond, wherein R denotes any substituted or unsubstituted alkyl or aryl group.

The phrase “substantially water-insoluble” as used in reference to an ester of a phosphorous oxo acid refers to a molecule that partitions into an organic phase in preference to an aqueous phase. It is to be understood that the phrase “substantially water-insoluble” encompasses but does not necessarily imply 0% aqueous solubility.

The term “quinone” refers to a molecule derived from an aromatic compound (e.g., benzene, naphthalene, anthracene, and the like, and combinations thereof) by conversion of an even number of —CH═ moieties into —C(═O)— groups together with whatever rearrangement of double bonds may be necessary to form one or a plurality of conjugated cyclic dione structures and/or substructures.

The term “hydroquinone” refers to a molecule derivable from a quinone by reduction (e.g., catechol is a hydroquinone derivable from 1,2-benzoquinone).

The phrases “natural oil,” “natural oil feedstock,” and the like refer to oils derived from plant or animal sources. As used herein, these phrases encompass natural oil derivatives as well, unless otherwise indicated.

The term “derivative” as used in reference to a substrate (e.g., a “functionalized derivative” of a carboxylic acid, such as 9-decenoic acid, etc.) refers to compounds and/or mixture of compounds derived from the substrate by any one or combination of methods known in the art, including but not limited to saponification, transesterification, esterification, amidification, amination, imide preparation, hydrogenation (partial or full), isomerization, oxidation, reduction, and the like, and combinations thereof.

The phrase “natural oil derivatives” refers to compounds and/or mixtures of compounds derived from a natural oil using any one or combination of methods known in the art, including but not limited to saponification, transesterification, esterification, amidification, amination, hydrogenation (partial or full), isomerization, oxidation, reduction, and the like, and combinations thereof.

The phrase “low-molecular-weight olefin” refers to any straight, branched, or cyclic olefin in the C₂ to C₃₀ range and/or any combination of such olefins. The phrase “low-molecular-weight olefin” encompasses mono-olefins, including but not limited to internal olefins, terminal olefins, and combinations thereof, as well as polyolefins, including but not limited to dienes, trienes, and the like, and combinations thereof. In some embodiments, the low-molecular-weight olefin is functionalized.

The term “ester” refers to compounds having a general formula R—COO—R′, wherein R and R′ denote any substituted or unsubstituted alkyl or aryl group. In some embodiments, the term “ester” refers to a group of compounds having a general formula as described above, wherein the compounds have different chain lengths.

The term “alkyl” refers to straight, branched, cyclic, and/or polycyclic aliphatic hydrocarbon groups, which optionally may incorporate one or more heteroatoms within their carbon-carbon backbones (e.g., so as to form ethers, heterocycles, and the like), and which optionally may be functionalized.

The phrase “residual metathesis catalyst” refers to a material left over from a metathesis reaction that is capable of participating in, catalyzing and/or otherwise promoting or facilitating dehydrogenation of an olefin metathesis product and/or isomerization of a carbon-carbon double bond even though the material itself may or may not still be capable of catalyzing a metathesis reaction. As used herein, the phrase “residual metathesis catalyst” encompasses wholly unreacted metathesis catalyst, partially reacted metathesis catalyst, and all manner of chemical entities derived from a metathesis catalyst over the course of a metathesis reaction, including but not limited to all manner of active or inactive intermediates (e.g., carbenes, metallocycles, etc.), degradation and/or decomposition products (e.g., metal hydrides, oxides, ligand fragments, etc.), metals, metal salts, metal complexes, and the like, and combinations thereof.

The phrase “hydrogen transfer agent” refers to a compound that is capable of participating in, catalyzing and/or otherwise promoting or facilitating hydrogen transfer in a molecule. Representative hydrogen transfer agents include but are not limited to dehydrogenation agents, hydrogenation agents (including but not limited to hydrogenation catalysts), and the like, and combinations thereof.

The term “passivate” as used in reference to a metal-containing material refers to any reduction in the activity of the metal-containing material vis-à-vis its ability and/or tendency to catalyze and/or otherwise participate in (e.g., via a stoichiometric chemical reaction, sequestration or the like), promote, and/or facilitate dehydrogenation of an olefin metathesis product and/or isomerization of a carbon-carbon double bond. It is to be understood that the term “passivate” encompasses but does not necessarily imply complete deactivation of a metal-containing material towards dehydrogenation and/or isomerization.

The phrase “conditions sufficient to passivate” as used in reference to the conditions under which a dehydrogenation suppression agent is added to a mixture comprising (i) olefin metathesis product and/or optionally functionalized olefin reactant, and (ii) metal-containing material refers to a variable combination of experimental parameters, which together result in the passivation of at least a portion of metal-containing material. The selection of these individual parameters lies well within the skill of the ordinary artisan in view of the guiding principles outlined herein, and will vary according to the target reduction in degree of dehydrogenation and/or isomerization that is being sought for a particular application. As used herein, the phrase “conditions sufficient to passivate” encompasses experimental parameters including but not limited to concentrations of reagents, the type of mixing and/or stirring provided (e.g., high-shear, low-intensity, etc.), reaction temperature, residence time, reaction pressure, reaction atmosphere (e.g., exposure to atmosphere vs. an inert gas, etc.), and the like, and combinations thereof.

The phrase “degree of isomerization” refers to an amount to which a carbon-carbon double bond in a molecule undergoes migration from its original position to a subsequent position (e.g., the degree to which an initially formed olefin metathesis product is converted into one or more non-identical isomers thereof). In some embodiments, the “degree of isomerization” refers to the degree to which an initially formed α-olefin metathesis product is converted into one or more internal isomers thereof under a given set of conditions (e.g., the amount of terminal-to-internal migration). In some embodiments, the “degree of isomerization” refers to the degree to which an olefin metathesis product containing an internal carbon-carbon double bond is converted into an α-olefin under a given set of conditions (e.g., the amount of internal-to-terminal migration). In some embodiments, the “degree of isomerization” refers to the degree to which an olefin metathesis product containing an internal carbon-carbon double bond is converted into one or more additional and non-identical internal isomers thereof under a given set of conditions (e.g., the amount of internal-to-internal migration). In some embodiments, the “degree of isomerization” refers to the degree to which an initially formed α-olefin metathesis product is converted into a different α-olefin under a given set of conditions (e.g., the amount of terminal-to-terminal migration). In some embodiments, the “degree of isomerization” refers to any combination of the amount of terminal-to-internal migration, the amount of internal-to-terminal migration, the amount of internal-to-internal migration, and/or the amount of terminal-to-terminal migration.

The term “attached” as used in reference to a solid support and a dehydrogenation suppression agent is to be understood broadly and without limitation to encompass a range of associative-type forces, including but not limited to covalent bonds, ionic bonds, physical and/or electrostatic attractive forces (e.g., hydrogen bonds, Van der Waals forces, etc.), and the like, and combinations thereof.

The term “paraffin” refers to hydrocarbon compounds having only single carbon-carbon bonds and having a general formula CₙH₂ₙ₊₂. In some embodiments, n is greater than 20.

The term “isomerizing” as used in reference to a “fuel composition” refers to the reaction and conversion of straight-chain hydrocarbon compounds, such as normal paraffins, into branched hydrocarbon compounds, such as iso-paraffins. As a representative and non-limiting example, n-pentane may be isomerized into a mixture of n-pentane, 2-methylbutane, and 2,2-dimethylpropane. Isomerization of normal paraffins may be used to improve the overall properties of a fuel composition. Additionally, isomerization may refer to the conversion of branched paraffins into further, more highly branched paraffins.

The term “yield” refers to the total weight of fuel produced from the metathesis and hydrogenation reactions. It may also refer to the total weight of the fuel following a separation step and/or isomerization reaction. It may be defined in terms of a yield %, wherein the total weight of the fuel produced is divided by the total weight of the natural oil feedstock and, in some embodiments, low-molecular-weight olefin, combined.

The term “fuel” and the phrase “fuel composition” refer to materials meeting certain specifications or a blend of components that are useful in formulating fuel compositions but, by themselves, do not meet all of the required specifications for a fuel.

The phrases “jet fuel” and “aviation fuel” refer to kerosene or naphtha-type fuel cuts, and/or military-grade jet fuel compositions. “Kerosene-type” jet fuel (including Jet A and Jet A-1) has a carbon number distribution between about 8 and about 16. Jet A and Jet A-1 typically have a flash point of at least approximately 38° C., an auto ignition temperature of approximately 210° C., a freeze point less than or equal to approximately −40° C. for Jet A and −47° C. for Jet A-1, a density of approximately 0.8 g/cc at 15° C., and an energy density of approximately 42.8-43.2 MJ/kg. “Naphtha-type” or “wide-cut” jet fuel (including Jet B) has a carbon number distribution between about 5 and about 15. Jet B typically comprises a flash point below approximately 0° C., an auto ignition temperature of approximately 250° C., a freeze point of approximately −51° C., a density of approximately 0.78 g/cc, and an energy density of approximately 42.8-43.5 MJ/kg. “Military grade” jet fuel refers to the Jet Propulsion or “JP” numbering system (JP-1, JP-2, JP-3, JP-4, JP-5, JP-6, JP-7, JP-8, etc.). Military grade jet fuels may comprise alternative or additional additives to have higher flash points than Jet A, Jet A-1, or Jet B in order to cope with heat and stress endured during supersonic flight.

The phrase “diesel fuel” refers to a hydrocarbon composition having a carbon number distribution between about 8 and about 25. Diesel fuels typically have a specific gravity of approximately 0.82-1.08 at 15.6° C. (60° F.) based on water having a specific gravity of 1 at 60° F. Diesel fuels typically comprise a distillation range between approximately 180-340° C. (356-644° F.). Additionally, diesel fuels have a minimum cetane index number of approximately 40.

As used herein, the phrase “carbon number distribution” refers to the range of compounds present in a composition, wherein each compound is defined by the number of carbon atoms present. As a non-limiting example, a naphtha-type jet fuel typically comprises a distribution of hydrocarbon compounds wherein a majority of those compounds have between 5 and 15 carbon atoms each. A kerosene-type jet fuel typically comprises a distribution of hydrocarbon compounds wherein a majority of those compounds have between 8 and 16 carbon atoms each. A diesel fuel typically comprises a distribution of hydrocarbon compounds wherein a majority of those compounds have between 8 and 25 carbon atoms each.

As used herein, the phrase “energy density” refers to the amount of energy stored in a given system per unit mass (MJ/kg) or per unit volume (MJ/L), where MJ refer to million Joules. As a non-limiting example, the energy density of kerosene- or naphtha-type jet fuel is typically greater than about 40 MJ/kg.

By way of general background, some olefin metathesis products and/or reactants, particularly in the presence of certain metal-containing materials (including but not limited to ones that facilitate hydrogen transfer), are—or can become (e.g., through initial olefin isomerization, etc.)—susceptible to dehydrogenation. Moreover, the dehydrogenation of certain metathesis products and/or reactants can lead to the formation of volatile organic compounds (VOCs), including but not limited to benzene—a highly undesirable and highly carcinogenic byproduct. For example, while neither desiring to be bound by any particular theory nor intending to limit in any measure the scope of the appended claims or their equivalents, it is presently believed that reactants and/or products that contain at least one methylene-interrupted polyolefin substructure (e.g., as may be found in certain polyunsaturated fatty acids and/or derivatives thereof, including but not limited to triglycerides) can form a CHD which, in the presence of a metal-containing material (e.g., residual metathesis catalyst from a metathesis reaction, hydrogenation catalyst added subsequent to a metathesis reaction in order to hydrogenate initially formed olefin metathesis product, and the like), can be dehydrogenated to produce benzene, thereby contaminating a desired metathesis product and producing an IARC Group 1 carcinogen. Representative methylene-interrupted polyolefins that can result in CHD and/or benzene formation in a metathesis reaction include but are not limited to 1,4-pentadiene, 1,4-hexadiene, 1,4-heptadiene, 1,4-octadiene, 1,4-nonadiene, 1,4-decadiene, 2,5-heptadiene, 2,5-octadiene, 2,5-nonadiene, 2,5-decadiene, 3,6-nonadiene, 3,6-decadiene, 1,4,6-octatriene, 1,4,7-octatriene, 1,4,6-nonatriene, 1,4,7-nonatriene, 1,4,6-decatriene, 1,4,7-decatriene, 2,5,8-decatriene, 18:2 and/or 18:3 fatty acids (e.g., linoleic acid, linolenic acid, etc.), 20:5 and/or 20:6 fatty acids (e.g., eicosapentaenoic acid, docosahexaenoic acid, etc.), and the like, and derivatives thereof, and combinations thereof.

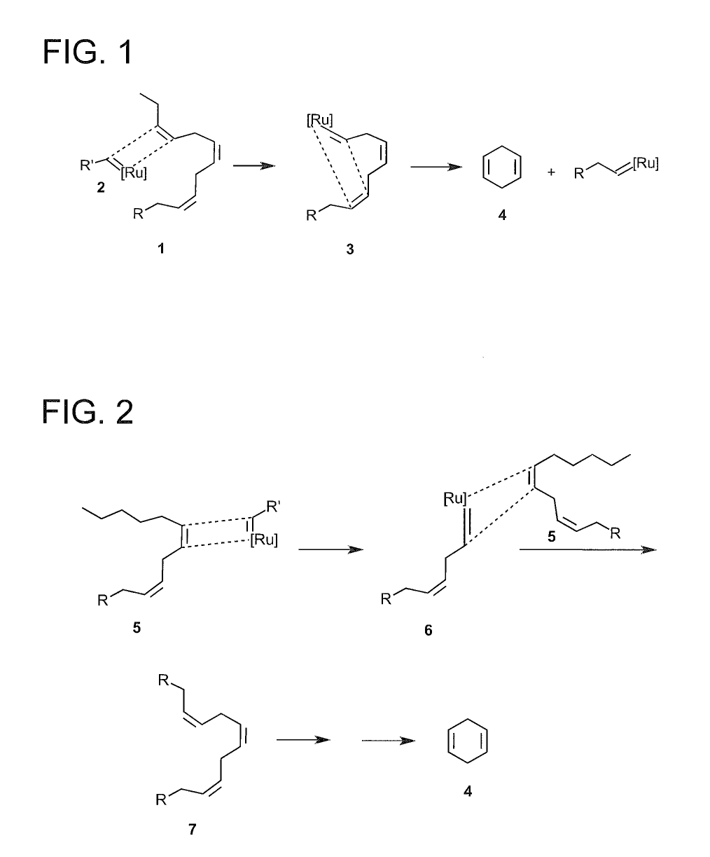

FIG. 1 shows one possible mechanistic pathway by which 1,4-CHD 4 can form in a metathesis reaction involving an 18:3 fatty acid and/or a derivative thereof 1. Similarly, FIG. 2 shows one possible mechanistic pathway by which 1,4-CHD 4 can form in a metathesis reaction involving an 18:2 fatty acid and/or a derivative thereof 5. In each of FIGS. 1 and 2, for the sake of clarity, only select portions of the molecules are shown, with remaining portions being depicted as generic residues R and R′. For simplicity, the carbon-carbon double bonds in FIGS. 1 and 2 are shown in the cis configuration, although trans carbon-carbon double bonds can react in similar fashion and are not meant to be excluded from this representative mechanistic scheme. In addition, in each of FIGS. 1 and 2, the broken lines are intended merely as visual aids to assist in tracking double bonds being reacted and/or formed, and are in no way intended to be indicative of actual mechanistic pathways.

As shown in FIG. 1, an 18:3 fatty acid starting material 1 (e.g., 9c12c15c α-linolenic acid) reacts with a ruthenium carbene catalyst 2 to form the ruthenium carbene intermediate 3. The ruthenium carbene intermediate 3, in turn, can undergo internal metathesis to provide 1,4-CHD 4. As shown in FIG. 2, an 18:2 fatty acid starting material 5 (e.g., 9c12c linoleic acid) reacts with the ruthenium carbene catalyst 2 to form a ruthenium carbene intermediate 6. The ruthenium carbene intermediate 6, in turn, can undergo metathesis with an additional molecule of starting material 5 to provide a polyolefin 7, which is structurally analogous to the starting material 1 shown in FIG. 1, and which can react similarly to 1 to form 1,4-CHD 4.

While neither desiring to be bound by any particular theory nor intending to limit in any measure the scope of the appended claims or their equivalents, it is presently believed that in the presence of metal-containing material, 1,4-CHD may initially undergo olefin isomerization to form 1,3-CHD prior to undergoing dehydrogenation to form benzene since the energy barrier to the dehydrogenation starting from the conjugated 1,3-isomer may be lower than from its non-conjugated 1,4-isomer.

It is to be understood that elements and features of the various representative embodiments described below may be combined in different ways to produce new embodiments that likewise fall within the scope of the present teachings.

By way of general introduction, a method for suppressing dehydrogenation in accordance with the present teachings comprises reacting an optionally functionalized olefin reactant in a metathesis reaction to form an olefin metathesis product, and providing a dehydrogenation suppression agent in admixture with (a) the olefin metathesis product and/or the optionally functionalized olefin reactant, and (b) a metal-containing material. In some embodiments, the adding is performed under conditions that are sufficient to passivate at least a portion of the metal-containing material. In some embodiments, non-passivated metal-containing material is configured to participate in, catalyze, promote, and/or facilitate dehydrogenation and/or isomerization of the optionally functionalized olefin reactant and/or the olefin metathesis product.

In some embodiments, the optionally functionalized olefin reactant and/or the olefin metathesis product comprises one or a plurality of substructures having a formula —CH═CH—CH₂—CH═CH—. In some embodiments, the optionally functionalized olefin reactant comprises a polyunsaturated fatty acid and/or a derivative thereof. Representative derivatives of polyunsaturated fatty acid include but are not limited to alcohols, esters, monoacylglycerides, diacylglycerides, triacylglycerides, and the like, and combinations thereof. In some embodiments, the derivative comprises an ester. In some embodiments, the derivative is selected from the group consisting of monoacylglycerides, diacylglycerides, triacylglycerides, and combinations thereof. In some embodiments, the derivative comprises a triacylglyceride.

In some embodiments, the optionally functionalized olefin reactant comprises an optionally functionalized low-molecular weight olefin. In some embodiments, the optionally functionalized olefin reactant comprises an optionally functionalized ester. In some embodiments, the optionally functionalized olefin reactant comprises a polyunsaturated hydrocarbon olefin and/or a derivative thereof. Representative optionally functionalized olefin reactants include but are not limited to 1,4-pentadiene, 1,4-hexadiene, 1,4-heptadiene, 1,4-octadiene, 1,4-nonadiene, 1,4-decadiene, 2,5-heptadiene, 2,5-octadiene, 2,5-nonadiene, 2,5-decadiene, 3,6-nonadiene, 3,6-decadiene, 1,4,6-octatriene, 1,4,7-octatriene, 1,4,6-nonatriene, 1,4,7-nonatriene, 1,4,6-decatriene, 1,4,7-decatriene, 2,5,8-decatriene, 18:2 and/or 18:3 fatty acids (e.g., linoleic acid, linolenic acid, etc.), 20:5 and/or 20:6 fatty acids (e.g., eicosapentaenoic acid, docosahexaenoic acid, etc.), and the like, and derivatives thereof, and combinations thereof.

In some embodiments, the optionally functionalized olefin reactant comprises an optionally functionalized polyunsaturated fatty acid and/or a derivative thereof, and the fatty acid comprises one or a plurality of methylene-interrupted diene substructures. In some embodiments, the fatty acid is selected from the group consisting of omega-3 fatty acids, omega-6 fatty acids, omega-9 fatty acids, and combinations thereof. In some embodiments, the fatty acid is selected from the group consisting of linoleic acid (18:2), linolenic acid (18:3; α- and/or γ-) eicosapentaenoic acid (20:5), docosahexaenoic acid (22:6), hexadecatrienoic acid (16:3), stearidonic acid (18:4), eicosatrienoic acid (20:3), eicosatetraenoic acid (20:4), heneicosapentaenoic acid (21:5), docosapentaenoic acid (22:5), tetracosapentaenoic acid (24:5), tetracosahexaenoic acid (24:6), eicosadienoic acid 20:2), dihomo-gamma-linolenic acid (20:3), arachidonic acid (20:4), docosadienoic acid (22:2), adrenic acid (22:4), tetracosatetraenoic acid (24:4), mead acid (20:3), and the like, and combinations thereof. In some embodiments, the fatty acid is selected from the group consisting of linoleic acid, linolenic acid, eicosapentaenoic acid, docosahexaenoic acid, and combinations thereof.

In some embodiments, the optionally functionalized olefin reactant comprises a natural oil. In some embodiments, the metathesis reaction that produced the olefin metathesis product comprises self-metathesis of a natural oil and/or a derivative thereof. In some embodiments, the metathesis reaction that produced the olefin metathesis product comprises cross-metathesis between a natural oil and/or a derivative thereof, and a low and/or a high molecular weight olefin. In some embodiments, the metathesis reaction that produced the olefin metathesis product comprises cross-metathesis between a natural oil and/or a derivative thereof, and a low molecular weight olefin. In some embodiments, the metathesis reaction that produced the olefin metathesis product comprises cross-metathesis between a natural oil and/or a derivative thereof, and a high molecular weight olefin.

Representative examples of natural oils for use in accordance with the present teachings include but are not limited to vegetable oils, algal oils, animal fats, tall oils (e.g., by-products of wood pulp manufacture), derivatives of these oils, and the like, and combinations thereof. Representative examples of vegetable oils for use in accordance with the present teachings include but are not limited to canola oil, rapeseed oil, coconut oil, corn oil, cottonseed oil, olive oil, palm oil, peanut oil, safflower oil, sesame oil, soybean oil, sunflower oil, high oleic sunflower oil, linseed oil, palm kernel oil, tung oil, jatropha oil, mustard oil, pennycress oil, camelina oil, hemp oil, castor oil, and the like, and combinations thereof. Representative examples of animal fats for use in accordance with the present teachings include but are not limited to lard, tallow, poultry fat, yellow grease, brown grease, fish oil, and the like, and combinations thereof. In some embodiments, the natural oil may be refined, bleached, and/or deodorized. In some embodiments, the natural oil is selected from the group consisting of canola oil, rapeseed oil, corn oil, cottonseed oil, peanut oil, sesame oil, soybean oil, sunflower oil, linseed oil, palm oil, tung oil, and combinations thereof.

Representative examples of natural oil derivatives for use in accordance with the present teachings include but are not limited to gums, phospholipids, soapstock, acidulated soapstock, distillate or distillate sludge, fatty acids, fatty acid alkyl esters (e.g., non-limiting examples such as 2-ethylhexyl ester, etc.), hydroxy-substituted variations thereof, and the like, and combinations thereof. In some embodiments, the natural oil derivative is a fatty acid methyl ester (FAME) derived from the glyceride of the natural oil.

In some embodiments, a plurality of olefin metathesis products is formed in the metathesis reaction, at least one of which is susceptible to dehydrogenation. In some embodiments, at least one of the olefin metathesis products is α,ω-di-functionalized. In some embodiments, at least one of the olefin metathesis products comprises a carboxylic acid moiety. In some embodiments, at least one of the olefin metathesis products comprises a terminal olefin and a carboxylic acid moiety. In some embodiments, at least one of the olefin metathesis products comprises an internal olefin and a carboxylic acid moiety. In some embodiments, at least one of the olefin metathesis products comprises a carboxylic ester moiety. In some embodiments, at least one of the olefin metathesis products comprises a terminal olefin and a carboxylic ester moiety. In some embodiments, at least one of the olefin metathesis products comprises an internal olefin and a carboxylic ester moiety. In some embodiments, at least one of the olefin metathesis products is selected from the group consisting of 9-decenoic acid, an ester of 9-decenoic acid, 9-undecenoic acid, an ester of 9-undecenoic acid, 9-dodecenoic acid, an ester of 9-dodecenoic acid, 1-decene, 2-dodecene, 3-dodecene, and combinations thereof. In some embodiments, the esters of 9-decenoic acid, 9-undecenoic acid, and 9-dodecenoic acid are alkyl esters, and, in some embodiments, methyl esters (e.g., methyl 9-decenoate, methyl 9-undecenoate, methyl 9-dodecenoate, etc.).

In some embodiments, at least one of the olefin metathesis products comprises at least one internal double bond, which in some embodiments is cis and in some embodiments is trans. In some embodiments, at least one of the olefin metathesis products comprises at least one terminal double bond and at least one internal double bond. In some embodiments, at least one of the olefin metathesis products comprises at least one terminal double bond and/or at least one internal double bond, and at least one additional functional group. In some embodiments, the at least one additional functional group is selected from the group consisting of carboxylic acids, carboxylic esters, mono-acylglycerides (MAGs), di-acylglycerides (DAGs), tri-acylglycerides (TAGs), and combinations thereof. In some embodiments, at least one of the olefin metathesis products is produced in a self-metathesis reaction. In some embodiments, at least one of the olefin metathesis products is produced in a cross-metathesis reaction. In some embodiments, at least one of the olefin metathesis products is a downstream derivative of a self-metathesis or cross-metathesis product (including but not limited to, for example, transesterification products, hydrolysis products, and the like, and combinations thereof). In some embodiments, at least one of the olefin metathesis products is produced in a metathesis reaction involving one or more previously formed olefin metathesis products (e.g., the production of 9-ODDAME from the cross-metathesis of 9-DAME and 9-DDAME—one or both of which is itself a product of a metathesis reaction).

In some embodiments, the at least one olefin metathesis product susceptible to dehydrogenation is a minor product of the metathesis reaction (e.g., a product that, in some embodiments, is formed in less than about 50% yield, in some embodiments less than about 40%, in some embodiments less than about 30%, in some embodiments less than about 20%, in some embodiments less than about 10%, and in some embodiments less than about 5%). In some embodiments, the at least one olefin metathesis product susceptible to dehydrogenation is cyclic. In some embodiments, the at least one olefin metathesis product susceptible to dehydrogenation and/or an isomer thereof is configured to form benzene via dehydrogenation. In some embodiments, the at least one olefin metathesis product susceptible to dehydrogenation comprises cyclohexadiene with representative cyclohexadienes including but not limited to 1,4-cyclohexadiene, 1,3-cyclohexadiene, and a combination thereof.

All manner of metathesis reactions are contemplated for use in accordance with the present teachings. Representative types of metathesis reactions include but are not limited to self-metathesis, CM, ROM, ROMP, RCM, ADMET, and the like, and combinations thereof. In some embodiments, the olefin metathesis product is produced in a metathesis reaction catalyzed by a ruthenium carbene complex. In some embodiments, the olefin metathesis product is produced in a metathesis reaction catalyzed by a molybdenum carbene complex. In some embodiments, the olefin metathesis product is produced in a metathesis reaction catalyzed by a tungsten carbene complex. In some embodiments, the metathesis reaction comprises ring-closing metathesis. In some embodiments, the metathesis reaction comprises self-metathesis of the optionally functionalized olefin reactant. In some embodiments, the optionally functionalized olefin reactant comprises a natural oil. In some embodiments, the metathesis reaction comprises cross-metathesis between the optionally functionalized olefin reactant and an optionally functionalized olefin co-reactant. In some embodiments, the optionally functionalized olefin reactant comprises a natural oil, and the optionally functionalized olefin co-reactant comprises a low-molecular weight olefin. In some embodiments, the optionally functionalized olefin reactant comprises a natural oil, and the optionally functionalized olefin co-reactant comprises a fatty acid methyl ester with representative FAMEs including but not limited to decenoic acid methyl esters (e.g., 9-DAME), undecenoic acid methyl esters (e.g., 9-UDAME), dodecenoic acid methyl esters (e.g., 9-DDAME), octadecenoic acid methyl esters (e.g., 9-ODDAME), and the like, and combinations thereof.

In some embodiments, the low-molecular-weight olefin is an “α-olefin” (aka “terminal olefin”) in which the unsaturated carbon-carbon bond is present at one end of the compound. In some embodiments, the low-molecular-weight olefin is an internal olefin. In some embodiments, the low-molecular-weight olefin is functionalized. In some embodiments, the low-molecular-weight olefin is a polyolefin. In some embodiments, the low-molecular-weight olefin comprises one or a plurality of substructures having a formula —CH═CH—CH₂—CH═CH—. In some embodiments, the low-molecular weight olefin is a C₂-C₃₀ olefin. In some embodiments, the low-molecular weight olefin is a C₂-C₃₀ α-olefin. In some embodiments, the low-molecular weight olefin is a C₂-C₂₅ olefin. In some embodiments, the low-molecular weight olefin is a C₂-C₂₅ α-olefin. In some embodiments, the low-molecular weight olefin is a C₂-C₂₀ olefin. In some embodiments, the low-molecular weight olefin is a C₂-C₂₀ α-olefin. In some embodiments, the low-molecular weight olefin is a C₂-C₁₅ olefin. In some embodiments, the low-molecular weight olefin is a C₂-C₁₅ α-olefin. In some embodiments, the low-molecular weight olefin is a C₂-C₁₄ olefin. In some embodiments, the low-molecular weight olefin is a C₂-C₁₄ α-olefin. In some embodiments, the low-molecular weight olefin is a C₂-C₁₀ olefin. In some embodiments, the low-molecular weight olefin is a C₂-C₁₀ α-olefin. In some embodiments, the low-molecular weight olefin is a C₂-C₈ olefin. In some embodiments, the low-molecular weight olefin is a C₂-C₈ α-olefin. In some embodiments, the low-molecular weight olefin is a C₂-C₆ olefin. In some embodiments, the low-molecular weight olefin is a C₂-C₆ α-olefin. Representative low-molecular-weight olefins include but are not limited to ethylene, propylene, 1-butene, 2-butene, isobutene, 1-pentene, 2-pentene, 3-pentene, 2-methyl-1-butene, 2-methyl-2-butene, 3-methyl-1-butene, cyclobutene, cyclopentene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, 1-hexene, 2-hexene, 3-hexene, cyclohexene, 1,4-pentadiene, 1,4-hexadiene, 1,4-heptadiene, 1,4-octadiene, 1,4-nonadiene, 1,4-decadiene, 2,5-heptadiene, 2,5-octadiene, 2,5-nonadiene, 2,5-decadiene, 3,6-nonadiene, 3,6-decadiene, 1,4,6-octatriene, 1,4,7-octatriene, 1,4,6-nonatriene, 1,4,7-nonatriene, 1,4,6-decatriene, 1,4,7-decatriene, 2,5,8-decatriene, and the like, and combinations thereof. In some embodiments, the low-molecular-weight olefin is an α-olefin selected from the group consisting of styrene, vinyl cyclohexane, and a combination thereof. In some embodiments, the low-molecular weight olefin is a mixture of linear and/or branched olefins in the C₄-C₁₀ range. In some embodiments, the low-molecular weight olefin is a mixture of linear and/or branched C₄ olefins (e.g., combinations of 1-butene, 2-butene, and/or iso-butene). In some embodiments, the low-molecular weight olefin is a mixture of linear and/or branched olefins in the higher C₁₁-C₁₄ range.

In some embodiments, the metathesis reaction that produced the olefin metathesis product comprises the reaction of two triglycerides present in a natural feedstock in the presence of a metathesis catalyst (self-metathesis), wherein each triglyceride comprises at least one carbon-carbon double bond, thereby forming a new mixture of olefins and esters that in some embodiments comprises a triglyceride dimer. In some embodiments, the triglyceride dimer comprises more than one carbon-carbon double bond, such that higher oligomers also can form. In some embodiments, the metathesis reaction that produced the olefin metathesis product comprises the reaction of an olefin (e.g., a low-molecular weight olefin) and a triglyceride in a natural feedstock that comprises at least one carbon-carbon double bond, thereby forming new olefinic molecules as well as new ester molecules (cross-metathesis).

The metal-containing material is not restricted and includes but is not limited to all manner of metal-containing materials configured to catalyze and/or otherwise facilitate or promote dehydrogenation and/or isomerization of the olefin metathesis product. In some embodiments, the metal-containing material comprises a transition metal. In some embodiments, the metal-containing material comprises residual metathesis catalyst from the metathesis reaction. In some embodiments, the metal-containing material comprises a hydrogen transfer agent. In some embodiments, the hydrogen transfer agent is selected from the group consisting of a hydrogenation catalyst, a dehydrogenation catalyst, and a combination thereof. In some embodiments, the metal-containing material is selected form the group consisting of residual metathesis catalyst, a hydrogenation catalyst, and a combination thereof.

In some embodiments, the metal-containing material comprises residual metathesis catalyst. In some embodiments, the residual metathesis catalyst comprises a transition metal. In some embodiments, the residual metathesis catalyst comprises ruthenium. In some embodiments, the residual metathesis catalyst comprises rhenium. In some embodiments, the residual metathesis catalyst comprises tantalum. In some embodiments, the residual metathesis catalyst comprises nickel. In some embodiments, the residual metathesis catalyst comprises tungsten. In some embodiments, the residual metathesis catalyst comprises molybdenum.

In some embodiments, the residual metathesis catalyst comprises a ruthenium carbene complex and/or an entity derived from such a complex. In some embodiments, the residual metathesis catalyst comprises a material selected from the group consisting of a ruthenium vinylidene complex, a ruthenium alkylidene complex, a ruthenium methylidene complex, a ruthenium benzylidene complex, and combinations thereof, and/or an entity derived from any such complex or combination of such complexes. In some embodiments, the residual metathesis catalyst comprises a ruthenium carbene complex comprising at least one phosphine ligand and/or an entity derived from such a complex. In some embodiments, the residual metathesis catalyst comprises a ruthenium carbene complex comprising at least one tricyclohexylphosphine ligand and/or an entity derived from such a complex. In some embodiments, the residual metathesis catalyst comprises a ruthenium carbene complex comprising at least two tricyclohexylphosphine ligands [e.g., (PCy₃)₂Cl₂Ru═CH—CH═C(CH₃)₂, etc.] and/or an entity derived from such a complex. In some embodiments, the residual metathesis catalyst comprises a ruthenium carbene complex comprising at least one imidazolidine ligand and/or an entity derived from such a complex. In some embodiments, the residual metathesis catalyst comprises a ruthenium carbene complex comprising an isopropyloxy group attached to a benzene ring and/or an entity derived from such a complex.

In some embodiments, the residual metathesis catalyst comprises a Grubbs-type olefin metathesis catalyst and/or an entity derived therefrom. In some embodiments, the residual metathesis catalyst comprises a first-generation Grubbs-type olefin metathesis catalyst and/or an entity derived therefrom. In some embodiments, the residual metathesis catalyst comprises a second-generation Grubbs-type olefin metathesis catalyst and/or an entity derived therefrom. In some embodiments, the residual metathesis catalyst comprises a first-generation Hoveda-Grubbs-type olefin metathesis catalyst and/or an entity derived therefrom. In some embodiments, the residual metathesis catalyst comprises a second-generation Hoveda-Grubbs-type olefin metathesis catalyst and/or an entity derived therefrom. In some embodiments, the residual metathesis catalyst comprises one or a plurality of the ruthenium carbene metathesis catalysts sold by Materia, Inc. of Pasadena, Calif. and/or one or more entities derived from such catalysts. Representative metathesis catalysts from Materia, Inc. for use in accordance with the present teachings include but are not limited to those sold under the following product numbers as well as combinations thereof: product no. C823 (CAS no. 172222-30-9), product no. C848 (CAS no. 246047-72-3), product no. C601 (CAS no. 203714-71-0), product no. C627 (CAS no. 301224-40-8), product no. C571 (CAS no. 927429-61-6), product no. C598 (CAS no. 802912-44-3), product no. C793 (CAS no. 927429-60-5), product no. C801 (CAS no. 194659-03-9), product no. C827 (CAS no. 253688-91-4), product no. C884 (CAS no. 900169-53-1), product no. C833 (CAS no. 1020085-61-3), product no. C859 (CAS no. 832146-68-6), product no. C711 (CAS no. 635679-24-2), product no. C933 (CAS no. 373640-75-6).

In some embodiments, the metal-containing material comprises a hydrogen transfer agent. In some embodiments, the hydrogen transfer agent comprises a hydrogenation catalyst. In some embodiments, the hydrogenation catalyst comprises a catalyst selected from the group consisting of homogeneous catalysts, heterogeneous catalysts, and combinations thereof. In some embodiments, the hydrogenation catalyst comprises a transition metal selected from the group consisting of nickel, palladium, platinum, rhodium, ruthenium, zinc, iron, cobalt, copper, and combinations thereof. In some embodiments, the metal-containing material comprises residual metathesis catalyst and a hydrogen transfer agent. In some embodiments, the metal-containing material comprises residual metathesis catalyst and a hydrogenation catalyst. In some embodiments, the hydrogenation catalyst is added to the mixture after the metathesis reaction.

Representative hydrogenation catalysts for use in accordance with the present teachings include but are not limited to those described in March's Advanced Organic Chemistry Reactions, Mechanisms, and Structure, 6^{th} Edition by Michael B. Smith and Jerry March (Wiley-Interscience: New Jersey, 2007, pages 1053-1074). Representative examples of such hydrogenation catalysts include but are not limited to Raney nickel, Urushibara nickel, palladium-on-charcoal, nickel boride, platinum metal and/or oxides thereof, rhodium, ruthenium, zinc oxide, chlorotris(triphenylphosphine)rhodium (Wilkinson's catalyst), (1,5-cyclooctadiene)(pyridine)(tricyclohexylphosphine)-Ir(I) hexafluorophosphate (Crabtree's catalyst), chlorotris(triphenylphosphine)hydridoruthenium(II), pentacyanocobaltate(II), colloidal palladium, polymer-bound ruthenium, polymer-incarcerated palladium, rhodium on mesoporous silica, nanoparticulate palladium in ionic liquid, and the like, and combinations thereof.

In some embodiments, the metal-containing material comprises a dehydrogenation catalyst. In some embodiments, the dehydrogenation catalyst comprises a transition metal. In some embodiments, the dehydrogenation catalyst comprises platinum supported on alumina. In some embodiments, the dehydrogenation catalyst comprises an oxidative dehydrogenation agent (including but not limited to a metal oxide). Representative dehydrogenation catalysts include but are not limited to those described, for example, in Industrial Organic Chemistry, Fourth, Completely Revised Edition by Klaus Weissermel and Hans-Jürgen Arpe (Wiley-VCH GmbH & Co. KGaA (2003; pages 39, 79, 112, 343, etc.).

In some embodiments, the dehydrogenation catalyst comprises a mixed metal oxide with representative metals including but not limited to molybdenum, vanadium, niobium, tellurium, magnesium, chromium, and/or aluminum. In some embodiments, the dehydrogenation catalyst comprises a phosphate of cerium/zirconium, zirconium, calcium/nickel, and/or alkaline earth/nickel. In some embodiments, the dehydrogenation catalyst comprises chromium, iron-chromium oxide, bismuth/molybdenum, tin/antimony, silver, copper, and/or combinations thereof.

In some embodiments, a dehydrogenation suppression agent in accordance with the present teachings passivates at least a portion of the metal-containing material. In some embodiments, the dehydrogenation suppression agent suppresses dehydrogenation of an olefin metathesis product and/or reactant. In some embodiments, the dehydrogenation suppression agent suppresses isomerization of an olefin metathesis product and/or reactant. In some embodiments, the dehydrogenation suppression agent suppresses dehydrogenation and isomerization of an olefin metathesis product and/or reactant. Representative dehydrogenation suppression agents for use in accordance with the present teachings include but are not limited to hydrogen transfer inhibitors.

In some embodiments, the dehydrogenation suppression agent comprises a quinone. While neither desiring to be bound by any particular theory nor intending to limit in any measure the scope of the appended claims or their equivalents, it is presently believed that a quinone dehydrogenation suppression agent can be added to a mixture prior to performing olefin metathesis as, in some embodiments, the quinone may not substantially impede and/or substantially modify the progression of the olefin metathesis reaction. Afterwards, the quinone dehydrogenation suppression agent can be removed from the metathesis mixture using a mass transfer process prior to sending the product mixture to various unit operations. Representative mass transfer processes for use in removing the dehydrogenation suppression agent include but are not limited to liquid-liquid extraction, crystallization, adsorption, stripping, and the like, and combinations thereof. Thus, in some embodiments, quinones provide additional flexibility with respect to the stage at which a dehydrogenation suppression agent in accordance with the present teachings is introduced into a reaction mixture (e.g., prior to metathesis vs. after metathesis).

In some embodiments, the dehydrogenation suppression agent comprises an electron-deficient quinone. As used herein, the phrase “electron-deficient” refers to substitution with one or a plurality of electron-withdrawing groups, with representative electron-withdrawing groups including but not limited to halogens (e.g., F, Cl, Br, and/or I), nitro, cyano, carbonyl groups (e.g., aldehydes, ketones, acids, esters, and the like, and combinations thereof), and the like, and combinations thereof. In some embodiments, the dehydrogenation suppression agent comprises an electron-rich quinone. As used herein, the phrase “electron-rich” refers to substitution with one or a plurality of electron-donating groups, with representative electron-donating groups including but not limited to hydroxyl, amines, alkyls, and the like, and combinations thereof.

In some embodiments, the dehydrogenation suppression agent is selected from the group consisting of optionally functionalized benzoquinones, optionally functionalized naphthoquinones, optionally functionalized anthraquinones, optionally functionalized hydroquinones, and the like, and combinations thereof. In some embodiments, the dehydrogenation suppression agent is selected from the group consisting of electron-deficient benzoquinones, electron-deficient naphthoquinones, electron-deficient anthraquinones, electron-deficient hydroquinones, and combinations thereof.

Representative quinones for use as dehydrogenation suppression agents in accordance with the present teachings include but are not limited to 1,2-benzoquinone, 1,4-benzoquinone, tetrachloro-p-benzoquinone, 2-chloro-1,4-benzoquinone, 2,6-dichloro-1,4-benzoquinone, difluoro-1,4-benzoquinone, trifluoro-1,4-benzoquinone, tetrafluoro-1,4-benzoquinone, 2,5-dichlorobenzoquinone, 2,3-dichloro-5,6-dicyano-1,4-benzoquinone, 1,2-naphthoquinone, 1,4-naphthoquinone, 2,6-naphthoquinone, 9,10-anthraquinone, 2-hydroxy-1,4-naphthoquinone, 2-chloro-1,4-naphthoquinone, 2,3-dichloro-1,4-naphthoquinone, 2-bromo-1,4-naphthoquinone, 2,3-dibromo-1,4-naphthoquinone, plastoquinone, phylloquinone, ubiquinone, 2,3-dihydroxy-9,10-anthraquinone, 2,6-dichloro-1,4-benzoquinone, tetrachloro-1,4-benzoquinone, 2,6-dimethoxy-1,4-benzoquinone, 2,6-di-tert-butyl-1,4-benzoquinone, and the like, and combinations thereof.

In some embodiments, the dehydrogenation suppression agent comprises a 1,4-benzoquinone. In some embodiments, the dehydrogenation suppression agent comprises an electron-deficient benzoquinone as described, for example, in JACS, 2005, 127, 17160-17161.

In some embodiments, the dehydrogenation suppression agent comprises a quinone derivative. In some embodiments, the dehydrogenation suppression agent comprises a hydroquinone. In some embodiments, the dehydrogenation suppression agent comprises a heterocyclic quinone derivative selected from the group consisting of pyridinones, thiopyranones, and the like, and combinations thereof.

In some embodiments, the dehydrogenation suppression agent comprises a radical inhibitor with a representative inhibitor including but not limited to tert-butyl hydroxytoluene.

In some embodiments, the dehydrogenation suppression agent comprises phosphorous. In some embodiments, the phosphorous-containing dehydrogenation suppression agent comprises a material selected from the group consisting of phosphine (PH₃), a phosphine (i.e., an organophosphorous compound), a phosphonium salt, a phosphine oxide, a phosphorous oxo acid, a salt of a phosphorous oxo acid, an ester of a phosphorous oxo acid, a derivative of a phosphorous oxo acid in which at least one P—H bond has been replaced by a P—C bond, a salt of the derivative, an ester of the derivative, and the like, and combinations thereof.

In some embodiments, the phosphorous-containing dehydrogenation suppression agent comprises a phosphine. In some embodiments, the phosphorous-containing dehydrogenation suppression agent comprises phosphine itself (PH₃) which, in some embodiments, can be dissolved in a non-polar solvent (e.g., an oil) at moderate to high concentrations. In some embodiments, the phosphorous-containing dehydrogenation suppression agent is selected from the group consisting of PH₃, primary phosphines, secondary phosphines, tertiary phosphines, and combinations thereof. In some embodiments, the phosphine is selected from the group consisting of optionally functionalized trialkylphosphines, optionally functionalized triarylphosphines, optionally functionalized mixed alkyl-aryl phosphines, and the like, and combinations thereof. In some embodiments, the phosphine comprises a structure P(R¹)(R²)(R³), wherein R¹, R², and R³ are alike or different. In some embodiments, R¹, R², and R³ are each independently selected from the group consisting of hydrogen, substituted or unsubstituted optionally functionalized C₁-C₁₀₀ alkyl, substituted or unsubstituted optionally functionalized aryl, and combinations thereof. In some embodiments, two of R¹, R², and R³ taken together may optionally form a ring with phosphorous. In some embodiments, covalent bonds may optionally exist between two or more of R¹, R², and R³. In some embodiments, the phosphine comprises at least one hydroxyl functionality.

In some embodiments, the phosphorous-containing dehydrogenation suppression agent comprises one or a plurality of the phosphorous-containing products sold by Rhodia, Inc. of Cranbury, N.J. and described, for example, in its brochure entitled Phosphorous Specialties (September 2008, pages 1-16).

Representative phosphines for use in accordance with the present teachings include but are not limited to phosphine, trimethylphosphine, triethylphosphine, tributylphosphine, triisopropylphosphine, triphenylphosphine, tricyclohexylphosphine, triallylphosphine, dimethylphenylphosphine, 2,2′-bis(diphenylphosphino)-1,1′-binaphthyl (BINAP), tris(4-methoxyphenyl)phosphine, tris(2,4,6-trimethoxyphenyl)phosphine, tris(hydroxymethyl)phosphine, tris(1-hydroxypropyl)phosphine, tris(3-hydroxypropyl)phosphine, dicyclohexylphosphine, tris(4-methylphenyl)phosphine, tris(2-methylphenyl)phosphine, tris(3-methylphenyl)phosphine, 1,1′-(1,2-ethanediyl)bis[1,1-diphenyl]phosphine, dibutylphosphine, (1,1-dimethylethyl)phosphine, bis(2-methylpropyl)phosphine, trioctylphosphine, tridodecylphosphine, 1,1′-(1,3-propanediyl)bis[1,1-diphenyl]phosphine, tricyclopentylphosphine, tris(phenylmethyl)phosphine, 1,1′-(1,4-butanediyl)bis[1,1-diphenyl]phosphine, diphenyl[2-(triethoxysilyl)ethyl]phosphine, (2,4,4-trimethylpentyl)phosphine, tris(3,5-dimethylphenyl)phosphine, mono isobutylphosphine, mono tert-butylphosphine, dicyclohexylphenylphosphine, mono cyclohexylphosphine, mono (2,4,4-trimethylpentyl)phosphine, di-isobutylphosphine, di-tert-butylphosphine, di-cyclopentylphosphine, dinorbornylphosphine, triisobutylphosphine, tricyclopentylphosphine, trihexylphosphine, tris(2-cyanoethyl)phosphine, 4,8-dimethyl-2-phosphabicyclo[3.3.1]nonane, 9-isobutyl-9-phosphabicyclo[3.3.1]nonane, 9-cyclohexyl-9-phosphabicyclo[3.3.1]nonane, 1,3,5,7-tetramethyl-2,4,8-trioxa-6-phenyl-6-phosphaadamantane, 2,2,6,6-tetramethyl-1-isobutyl-4-phosphorinanone, 1,2-(bis-isobutylphosphino)ethane, and the like, and combinations thereof. In some embodiments, the phosphine comprises tris(hydroxymethyl)phosphine.

In some embodiments, the phosphorous-containing dehydrogenation suppression agent comprises a phosphonium salt. In some embodiments, the phosphonium salt comprises a structure selected from the group consisting of [⁺P(R¹)(R²)(R³)(R⁴)]X⁻, [⁺P(R¹)(R²)(R³)(R⁴)]₂X²⁻, and a combination thereof, wherein R¹, R², R³, and R⁴ are alike or different and wherein X represents an anion. In some embodiments, R¹, R², R³, and R⁴ are each independently selected from the group consisting of hydrogen, substituted or unsubstituted optionally functionalized C₁-C₁₀₀ alkyl, substituted or unsubstituted optionally functionalized aryl, and combinations thereof. In some embodiments, two of R¹, R², R³, and R⁴ taken together may optionally form a ring with phosphorous. In some embodiments, covalent bonds may optionally exist between two or more of R¹, R², R³, and R⁴.

In some embodiments, the phosphorous-containing dehydrogenation suppression agent comprises one or a plurality of the phosphonium salts sold under the tradename CYPHOS by Cytec Industries, Inc. of Woodland Park, N.J.

Representative phosphonium salts for use in accordance with the present teachings include but are not limited to tetrakis(hydroxymethyl)phosphonium sulfate, tetrakis(hydroxymethyl)phosphonium chloride, tributylmethylphosphonium iodide, tetrabutylphosphonium iodide, triphenylmethylphosphonium iodide, triphenyl-propylphosphonium bromide, triphenylbenzylphosphonium chloride, tetrabutylphosphonium bromide, tetrabutylphosphonium chloride, tetradecyl(tributyl)phosphonium chloride (CYPHOS 3453W), hexadecyl(tributyl)phosphonium bromide (CYPHOS 3472P), tetraoctylphosphonium bromide (CYPPHOS 482), and the like, and combinations thereof. Representative phosphonium cations for use in accordance with the present teachings include but are not limited to tetrakis(hydroxymethyl)phosphonium, tributylmethylphosphonium, tetra-n-butylphosphonium, triphenylmethylphosphonium, triphenyl-propylphosphonium, triphenylbenzylphosphonium, and the like, and combinations thereof.

In some embodiments, the phosphorous-containing dehydrogenation suppression agent comprises a phosphine oxide. In some embodiments, the phosphine oxide comprises a primary phosphine oxide. In some embodiments, the phosphine oxide comprises a secondary phosphine oxide. In some embodiments, the phosphine oxide comprises a tertiary phosphine oxide. In some embodiments, the phosphine oxide comprises a structure P(═O)(R¹)(R²)(R³), wherein R¹, R², and R³ are alike or different. In some embodiments, R¹, R², and R³ are each independently selected from the group consisting of hydrogen, substituted or unsubstituted optionally functionalized C₁-C₁₀₀ alkyl, substituted or unsubstituted optionally functionalized aryl, and combinations thereof. In some embodiments, two of R¹, R², and R³ taken together may optionally form a ring with phosphorous. In some embodiments, covalent bonds may optionally exist between two or more of R¹, R², and R³. In some embodiments, the phosphine oxide comprises at least one hydroxyl functionality.

In some embodiments, the phosphorous-containing dehydrogenation suppression agent comprises one or a plurality of the phosphine oxides sold under the tradename CYANEX by Cytec Industries, Inc. of Woodland Park, N.J.

Representative phosphine oxides for use in accordance with the present teachings include but are not limited to tris(hydroxymethyl)phosphine oxide, tricyclohexylphosphine oxide, triphenylphosphine oxide, trimethylphosphine oxide, trioctylphosphine oxide, tributylphosphine oxide, tripropylphosphine oxide, (chloromethyl)dimethylphosphine oxide, trihexylphosphine oxide, tris(chloromethyl)phosphine oxide, tris(3-hydroxypropyl)phosphine oxide, trishydroxypropylphosphine oxide, bis(2,4,4-trimethylpentyl)phosphinic acid (CYANEX 272), trioctylphosphine oxide (CYANEX 921), mixed hexyl/octyl trialkylphosphine oxides (CYANEX 923) and the like, and combinations thereof.

In some embodiments, the phosphorous-containing dehydrogenation suppression agent comprises a phosphorous oxo acid and/or a salt thereof. In some embodiments, the phosphorous oxo acid comprises a higher acid. In some embodiments, the phosphorous oxo acid comprises a lower acid. Representative phosphorous oxo acids for use in accordance with the present teachings include but are not limited to those described in F. A. Cotton and G. Wilkinson's Advanced Inorganic Chemistry, Fifth Edition, New York: John Wiley & Sons, 1988, pages 382-443. By way of illustration, representative phosphorous oxo acids include but are not limited to phosphorous acid (H₃PO₃, aka “phosphonic acid”), phosphinic acid (H₃PO₂, aka “hypophosphorous acid), phosphoric acid (H₃PO₄, aka “orthophosphoric acid), pyrophosphoric acid (H₄P₂O₇), polyphosphoric acids, ultraphosphonic acid (H₂P₄O₁₁), di- and polyacids of phosphorous in lower formal oxidation states that comprise P—H and/or P—P bonds, and the like, and salts and anions thereof, and the like, and combinations thereof.

In some embodiments, the dehydrogenation suppression agent comprises phosphorous acid. Since phosphorous acid has a melting point of 73.6° C. and is typically a solid at room temperature, in some embodiments in accordance with the present teachings, neat phosphorous acid (i.e., in substantially solid form) is added to the mixture that comprises (i) an olefin metathesis product and/or optionally functionalized olefin reactant, and (ii) metal-containing material. In some embodiments, the dehydrogenation suppression agent comprises a solution of phosphorous acid. In some embodiments, the solution is aqueous. It is to be understood that the concentration of phosphorous acid is not restricted, and that all manner of concentrations are contemplated for use in accordance with the present teachings. In some embodiments, the dehydrogenation suppression agent comprises an aqueous solution of phosphorous acid in a concentration of between about 0.05 wt % and about 70 wt %. In some embodiments, the dehydrogenation suppression agent comprises an aqueous solution of phosphorous acid in a concentration of between about 0.1 wt % and about 70 wt %. In some embodiments, the dehydrogenation suppression agent comprises an aqueous solution of phosphorous acid in a concentration of between about 1 wt % and about 70 wt %. In some embodiments, the dehydrogenation suppression agent comprises an aqueous solution of phosphorous acid in a concentration of between about 5 wt % and about 50 wt %. In some embodiments, the dehydrogenation suppression agent comprises an aqueous solution of phosphorous acid in a concentration of between about 7 wt % and about 15 wt %. In some embodiments, the dehydrogenation suppression agent comprises an aqueous solution of phosphorous acid in a concentration of between about 1 and 50 wt %.

In alternative embodiments, the dehydrogenation suppression agent comprises an organic rather than aqueous solution of phosphorous acid. Representative organic solvents for use in forming organic solutions of phosphorous acid include but are not limited to alcohols (e.g., methanol, ethanol, etc.), acetonitrile, ethylene glycol, glycerol, glymes, polyethylene glycols, ionic liquids (i.e., salts in a liquid state) including but not limited to salts of 1-butyl-3-methylimidazolium (BMIM) (e.g., [BMIM][BF₄], [BMIM][PF₆], [BMIM][SbF₆], [BMIM][OTf], [BMIM][NTf₂], [MeACHTUNGTRENNUNG(C₂H₄O)₃MIM][BF₄], and the like, and combinations thereof.

In some embodiments, the dehydrogenation suppression agent comprises phosphorous acid and is attached to a solid support (e.g., silica gel). In some embodiments, the solid support comprises one or more polar functional groups. Representative solid supports for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof.

In some embodiments, the dehydrogenation suppression agent comprises phosphinic acid. However, since phosphinic acid and its salts are designated as a List I precursor chemical by the United States Drug Enforcement Administration (DEA)—thereby subjecting its handlers within the United States to stringent regulatory controls pursuant to the Controlled Substances Act and 21 CFR §§1309 and 1310—in some embodiments, the dehydrogenation suppression agent comprises phosphorous acid rather than phosphinic acid.

For embodiments in which the dehydrogenation suppression agent comprises phosphinic acid, the phosphinic acid can be added to a mixture in accordance with the present teachings in neat (i.e., in substantially solid form) and/or solution form. Since phosphinic acid has a melting point of 26.5° C., it may or may not be a solid at room temperature.

In some embodiments, the dehydrogenation suppression agent comprises a solution of phosphinic acid. In some embodiments, the solution is aqueous. It is to be understood that the concentration of phosphinic acid is not restricted, and that all manner of concentrations are contemplated for use in accordance with the present teachings. Typically, phosphinic acid is commercially available as a 50 wt % aqueous solution. In some embodiments, the dehydrogenation suppression agent comprises an aqueous solution of phosphinic acid in a concentration of between about 0.05 wt % and about 50 wt %. In some embodiments, the dehydrogenation suppression agent comprises an aqueous solution of phosphinic acid in a concentration of between about 0.1 wt % and about 50 wt %. In some embodiments, the dehydrogenation suppression agent comprises an aqueous solution of phosphinic acid in a concentration of between about 1 wt % and about 50 wt %. In some embodiments, the dehydrogenation suppression agent comprises an aqueous solution of phosphinic acid in a concentration of about 50 wt %. In alternative embodiments, the dehydrogenation suppression agent comprises an organic rather than aqueous solution of phosphinic acid. Representative organic solvents for use in forming organic solutions of phosphinic acid include but are not limited to alcohols (e.g., methanol, ethanol, etc.), acetonitrile, ethylene glycol, glycerol, glymes, polyethylene glycols, ionic liquids (i.e., salts in a liquid state) including but not limited to salts of 1-butyl-3-methylimidazolium (BMIM) (e.g., [BMIM][BF₄], [BMIM][PF₆], [BMIM][SbF₆], [BMIM][OTf], [BMIM][NTf₂], [MeACHTUNGTRENNUNG(C₂H₄O)₃MIM][BF₄], and the like, and combinations thereof.

In some embodiments, the dehydrogenation suppression agent comprises phosphinic acid and is attached to a solid support (e.g., silica gel). In some embodiments, the solid support comprises one or more polar functional groups. Representative solid supports for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof.

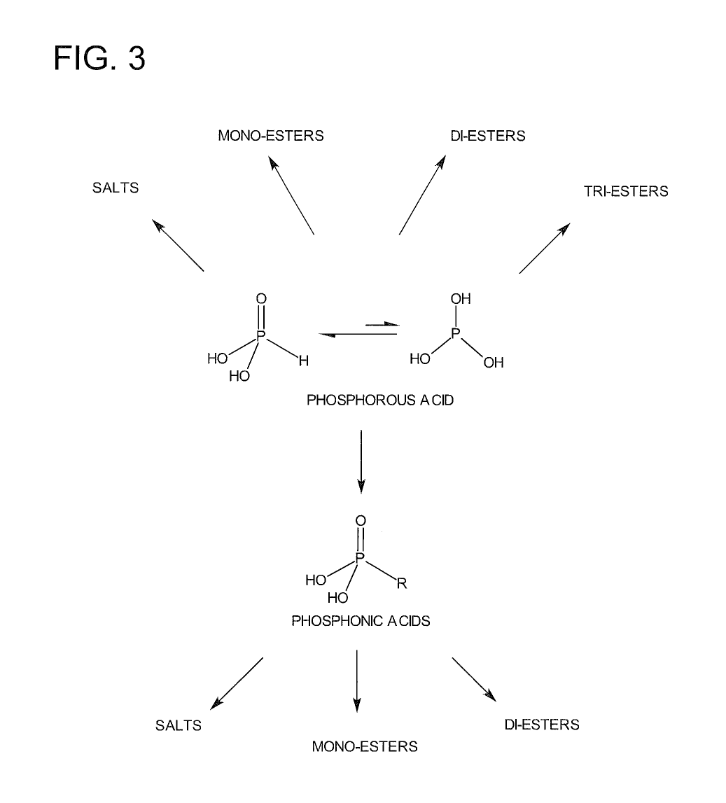

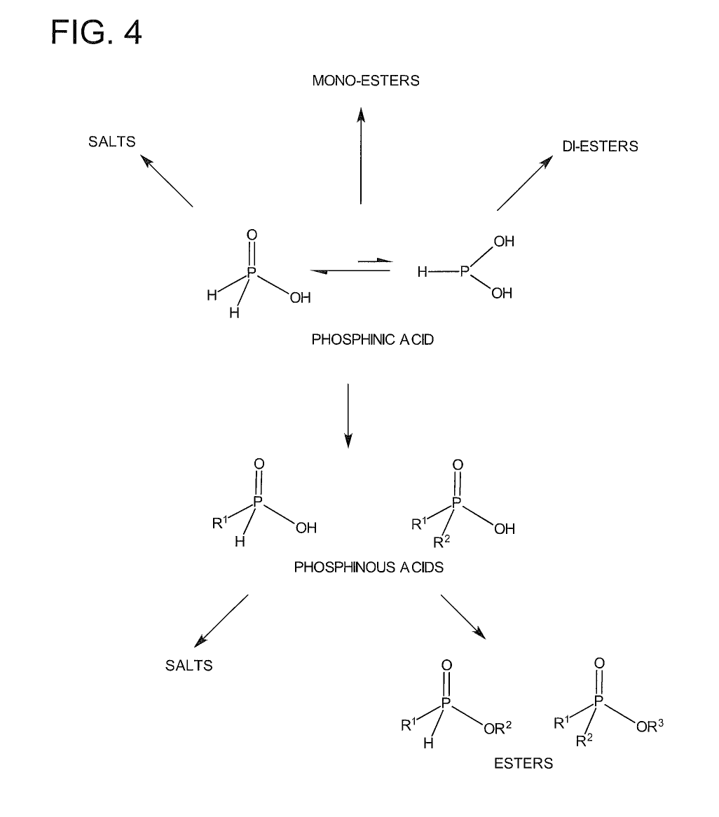

In some embodiments, the phosphorous-containing dehydrogenation suppression agent comprises an ester of a phosphorous oxo acid. In some embodiments, the phosphorous oxo acid comprises phosphorous acid and, as shown in FIG. 3, the ester of the phosphorous acid is selected from the group consisting of mono-esters, di-esters, tri-esters, and combinations thereof. In some embodiments, the phosphorous oxo acid comprises phosphinic acid and, as shown in FIG. 4, the ester of the phosphinic acid is selected from the group consisting of mono-esters, di-esters, and combinations thereof.

In some embodiments, the phosphorous-containing dehydrogenation suppression agent comprises a mono-ester of phosphorous acid. Representative mono-esters of phosphorous acid for use in accordance with the present teachings include but are not limited to phenylphosphoric acid, which is described in Eur. J. Chem., 2007, 918-924. In some embodiments, the phosphorous-containing dehydrogenation suppression agent comprises a tri-ester of phosphorous acid. In some embodiments, the phosphorous-containing dehydrogenation suppression agent comprises a phosphite ester.

In some embodiments, the phosphite ester comprises a structure P(OR¹)(OR²)(OR³), wherein R¹, R², and R³ are alike or different and are each independently selected from the group consisting of substituted or unsubstituted optionally functionalized C₁-C₁₀₀ alkyl, substituted or unsubstituted optionally functionalized aryl, and combinations thereof. In some embodiments, two of OR¹, OR², and OR³ taken together may optionally form a ring with phosphorous. In some embodiments, covalent bonds may optionally exist between two or more of R¹, R², and R³.

In some embodiments, the phosphite ester is selected from the group consisting of aryl organophosphites, alkyl organophosphites, aryl-alkyl mixed organophosphites, and combinations thereof. In some embodiments, the phosphite ester comprises one or a plurality of the high molecular weight phosphites commercially available from Dover Chemical Corporation of Dover, Ohio and/or Galata Chemicals of Southbury, Conn. Representative phosphites from Dover Chemical Corporation for use in accordance with the present teachings include both liquids and solids, and include but are not limited to those sold under the following product names as well as combinations thereof: trisnonylphenyl phosphite (DOVERPHOS® 4), trisnonylphenyl phosphite (+0.75% triisopropanolamine) (DOVERPHOS® 4-HR), trisnonylphenyl phosphite (+1.0% triisopropanolamine) (DOVERPHOS® 4-HR Plus), trisnonylphenyl phosphite containing maximum residual nonylphenol of 0.1% (DOVERPHOS® HIPURE 4), trisnonylphenyl phosphite (+0.75% triisopropanolamine) containing maximum residual nonylphenol of 0.1% (DOVERPHOS® HIPURE 4-HR), diphenyl phosphite (DOVERPHOS® 213), triphenyl phosphite (DOVERPHOS® 10), phenyl diisodecyl phosphite (DOVERPHOS® 7), diphenyl isodecyl phosphite (DOVERPHOS® 8), diphenyl isooctyl phosphite (DOVERPHOS® 9), tetraphenyl dipropyleneglycol diphosphite (DOVERPHOS® 11), poly (dipropyleneglycol) phenyl phosphite (DOVERPHOS® 12), C₁₂-C₁₅ alkyl bisphenol A phosphite (DOVERPHOS® 613), C₁₀ alkyl bisphenol A phosphite (DOVERPHOS® 675), triisodecyl phosphite (DOVERPHOS® 6), tris(tridecyl) phosphite (DOVERPHOS® 49), trilauryl phosphite (DOVERPHOS® 53), tris(dipropylene glycol) phosphite (DOVERPHOS® 72), dioleyl hydrogen phosphite (DOVERPHOS® 253), tris(2,4-di-tert-butylphenyl) phosphite (DOVERPHOS® S-480), distearyl pentaerythritol diphosphite (DOVERPHOS® S-680), distearyl pentaerythritol diphosphite (+triisopropanolamine) (DOVERPHOS® S-682), bis(2,4-dicumylphenyl) pentaerythritol diphosphite (DOVERPHOS® S-9228), and the like, and combinations thereof. Representative phosphites from Galata Chemicals for use in accordance with the present teachings include both liquids and solids, and include but are not limited to those sold under the following product names as well as combinations thereof: tris (nonylphenyl) phosphite, diphenyl phosphite, triphenyl phosphite, phenyl diisodecyl phosphite, diphenyl isodecyl phosphite, dodecyl nonylphenol phosphite blend, triisodecyl phosphite, triisotridecyl phosphite, 2-ethylhexyl diphenyl phosphite, poly (dipropylene glycol) phenyl phosphite, tetraphenyl dipropyleneglycol diphosphite, trilauryl phosphite, phenyl neopentylene glycol phosphite, heptakis (dipropyleneglycol) triphosphite, trilauryl trithio phosphite, diphenyl tridecyl phosphite, tris (dipropyleneglycol) phosphite, poly 4,4′ isopropylidenediphenol-C10 alcohol phosphite, 4,4′ isopropylidenediphenol-C12-15 alcohol phosphite, and the like, and combinations thereof.

In some embodiments, the phosphorous-containing dehydrogenation suppression agent comprises a C₁₂-C₁₅ alcohol phosphite. In some embodiments, the phosphorous-containing dehydrogenation suppression agent comprises bis(2,4,4-trimethylpentyl)phosphinic acid (CYANEX®272). In some embodiments, the phosphorous-containing dehydrogenation suppression agent comprises mono(2,4,4-trimethylpentyl)phosphonic acid (CYPHOS®SM 194).

In some embodiments, the phosphorous-containing dehydrogenation suppression agent comprises a substantially water-insoluble ester of a phosphorous oxo acid which, in some embodiments, may not partition to a significant degree in a polar solvent. It is to be understood that under a give set of biphasic conditions, a “substantially water-insoluble” ester of a phosphorous oxo acid may partition to some extent into the aqueous phase rather than the organic phase (albeit in an amount that is less than about 50 wt %, in some embodiments less than about 40 wt %, in some embodiments less than about 35 wt %, in some embodiments less than about 30 wt %, in some embodiments less than about 25 wt %, in some embodiments less than about 20 wt %, in some embodiments less than about 15 wt %, in some embodiments less than about 10 wt %, in some embodiments less than about 5 wt %, in some embodiments less than about 3 wt %, and in some embodiments less than about 1 wt %).

In some embodiments, the phosphorous-containing dehydrogenation suppression agent comprises a derivative of a phosphorous oxo acid in which at least one P—H bond has been replaced by a P—C bond and/or salts and/or esters of the derivative. In some embodiments, the phosphorous oxo acid comprises phosphorous acid and, as shown in FIG. 3, the derivative comprises a phosphonic acid. In some embodiments, the ester of the phosphonic acid derivative is selected from the group consisting of mono-esters, di-esters, and combinations thereof. In some embodiments, the ester comprises a phosphonate. In some embodiments, the ester comprises one or a plurality of the phosphonates commercially available from Thermphos International BV (Vlissingen, The Netherlands) and sold under the tradename DEQUEST. Representative phosphonates from Thermphos for use in accordance with the present teachings include but are not limited to those sold under the following product names as well as combinations thereof: amino trimethylene phosphonic acid and salts thereof (DEQUEST® 2000, DEQUEST® 2000EG, DEQUEST® 2000LC, DEQUEST® 2006), 1-hydroxyethylidene-1,1-diphosphonic acid and salts thereof (DEQUEST® 2010, DEQUEST® 2010CS, DEQUEST® 2010LA, DEQUEST® 2010LC, DEQUEST® 2014, DEQUEST® 2016, DEQUEST® 2016D, DEQUEST® 2016DG), DEQUEST® 2046, DEQUEST® 2047, DEQUEST® 2047G, diethylenetriamine penta(methylene phosphonic acid) and salts thereof (DEQUEST® 2060S, DEQUEST® 2066, DEQUEST® 2066A, DEQUEST® 2066C2), a proprietary polyamino phosphonic acid (DEQUEST® 2086), bis(hexamethylene triamine penta(methylene phosphonic acid)) and salts thereof (DEQUEST® 2090), diethylene triamine penta(methylene phosphonic acid) and salts thereof (DEQUEST® 4066), DEQUEST® 4266D, DEQUEST® 6004, and the like, and combinations thereof.

In some embodiments, the phosphorous oxo acid comprises phosphinic acid and, as shown in FIG. 4, the derivative of the phosphinic acid in which at least one P—H bond has been replaced by a P—C bond comprises a phosphinous acid. In some embodiments, the phosphinous acid is selected from the group consisting of R¹HP(O)OH, R²R³P(O)OH, and a combination thereof, wherein R¹, R², and R³ are alike or different and are each independently selected from the group consisting of substituted or unsubstituted optionally functionalized C₁-C₁₀₀ alkyl, substituted or unsubstituted optionally functionalized aryl, and combinations thereof, wherein a covalent bond may exist between R² and R³, such that when R² and R³ are taken together, a bidentate ligand to phosphorous is formed. In some embodiments, the ester of the phosphinous acid comprises a structure selected from the group consisting of R¹HP(O)OR², R³R⁴P(O)OR⁵, and a combination thereof, wherein R¹, R², R³, R⁴, and R⁵ are alike or different and are each independently selected from the group consisting of substituted or unsubstituted optionally functionalized C₁-C₁₀₀ alkyl, substituted or unsubstituted optionally functionalized aryl, and combinations thereof, wherein a covalent bond may exist between R¹ and R², such that when R¹ and R² are taken together, a bidentate ligand to phosphorous is formed, and wherein covalent bonds may optionally exist between two or more of R³, R⁴, and R⁵, such that when two or more of R³, R⁴, and R⁵ are taken together, a bidentate or tridentate ligand to phosphorous is formed.

In some embodiments, the dehydrogenation suppression agent is attached to a solid support (e.g., silica gel) and comprises (i) a salt and/or an ester of a phosphorous oxo acid, and/or (ii) a derivative of the phosphorous oxo acid in which at least one P—H bond has been replaced by a P—C bond, and/or (iii) a salt and/or an ester of the derivative. In some embodiments, the solid support comprises one or more polar functional groups. Representative solid supports for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof.

In some embodiments, the dehydrogenation suppression agent comprises nitrogen. In some embodiments, the nitrogen-containing dehydrogenation suppression agent comprises a material selected from the group consisting of ammonia, primary amines, secondary amines, tertiary amines, ammonium salts, polyamines, nitric acid, and the like, and combinations thereof.

In some embodiments, the nitrogen-containing dehydrogenation suppression agent comprises a primary amine. In some embodiments, the primary amine is selected from the group consisting of optionally functionalized alkyl amines, optionally functionalized aryl amines, and the like, and combinations thereof. In some embodiments, the primary amine comprises a structure having a formula NH₂R, wherein R is selected from the group consisting of substituted or unsubstituted optionally functionalized C₁-C₁₀₀ alkyl, substituted or unsubstituted optionally functionalized aryl, and combinations thereof.

Representative primary amines for use in accordance with the present teachings include but are not limited to methylamine, ethylamine, n-propylamine, iso-propylamine, n-butylamine, sec-butylamine, iso-butylamine, tert-butylamine, n-pentylamine, 1-amino-2-methylbutane, neo-pentylamine, pentan-3-amine, 2-methylbutan-2-amine, 3-methylbutan-2-amine, iso-pentylamine, 3-methylbutan-2-amine, ethylpropylamine, 3-methylbutan-2-amine, pentan-2-amine, 2-methylbutylamine, n-hexylamine, 2-ethylbutylamine, 3,3-dimethylbutan-2-amine, 1,3-dimethylbutylamine, 4-methylpentan-1-amine, 3-methylpentan-2-amine, 2,3-dimethylbutan-1-amine, 1,1-dimethylbutylamine, 3-methylpentan-3-amine, hexan-3-amine, 2-methylpentan-3-amine, 3-methylpentan-1-amine, 3,3-dimethylbutan-2-amine, cyclopropylamine, cyclobutylamine, cyclopentylamine, cyclohexylamine, aniline, 2-methoxyethylamine, 2-amino-2-hydroxymethyl-propane-1,3-diol, and the like, and combinations thereof.

In some embodiments, the nitrogen-containing dehydrogenation suppression agent comprises a secondary amine. In some embodiments, the secondary amines is selected from the group consisting of optionally functionalized alkyl amines, optionally functionalized aryl amines, optionally functionalized mixed alkyl-aryl amines, and the like, and combinations thereof. In some embodiments, the secondary amine comprises a structure having a formula NHR¹R², wherein R¹ and R² are alike or different and are each independently selected from the group consisting of substituted or unsubstituted optionally functionalized C₁-C₁₀₀ alkyl, substituted or unsubstituted optionally functionalized aryl, and combinations thereof. In some embodiments, R¹ and R² taken together may optionally form a ring with nitrogen. In some embodiments, covalent bonds may optionally exist between R¹ and R².

Representative secondary amines for use in accordance with the present teachings include but are not limited to dimethylamine, diethylamine, di-n-propylamine, ethyl(iso-propyl)amine, di-n-butylamine, di-tert-butylamine, di-sec-butylamine, di-iso-butylamine, N-methyl-2-butanamine, methyl(ethyl)amine, butyl(methyl)amine, tert-butyl(methyl)amine, methyl(iso-butyl)amine, butyl(ethyl)amine, tert-butyl(ethyl)amine, methyl(iso-amyl)amine, 1-ethylpropyl(methyl)amine, sec-butyl(ethyl)amine, methyl(2-methylbutan-2-yl)amine, methyl(2-methylbutyl)amine, propyl(iso-propyl)amine, 1,2-dimethylpropyl(methyl)amine, 2,2-dimethylpropyl(methyl)amine, N,N-diisopropylamine, methyl(pentyl)amine, pyrrolidine, piperidine, and the like, and combinations thereof.

In some embodiments, the nitrogen-containing dehydrogenation suppression agent comprises a tertiary amine. In some embodiments, the tertiary amine is selected from the group consisting of optionally functionalized alkyl amines, optionally functionalized aryl amines, optionally functionalized mixed alkyl-aryl amines, and combinations thereof. In some embodiments, the tertiary amine comprises a structure having a formula NR¹R²R³, wherein R¹, R², and R³ are alike or different and are each independently selected from the group consisting of substituted or unsubstituted optionally functionalized C₁-C₁₀₀ alkyl, substituted or unsubstituted optionally functionalized aryl, and combinations thereof. In some embodiments, two of R¹, R², and R³ taken together may optionally form a ring with nitrogen. In some embodiments, covalent bonds may optionally exist between two or more of R¹, R², and R³.

Representative tertiary amines for use in accordance with the present teachings include but are not limited to trimethylamine, triethylamine, tripropylamine, triphenylamine, N-methyldiphenylamine, N,N-dimethylethylamine, N,N-diethylmethylamine, N,N-diethylpropylamine, N,N-dimethylisopropylamine, tert-butyldimethylamine, N,N-dimethylaniline, N,N-dimethylcyclohexylamine, N-methylpyrrolidine, N-methylpiperidine, and the like, and combinations thereof.

In some embodiments, the nitrogen-containing dehydrogenation suppression agent comprises an ammonium salt. In some embodiments, the ammonium salt comprises an ammonium cation selected from the group consisting of ammonium ion itself (NH₄⁺) primary ammonium cations [(NH₃R)⁺], secondary ammonium cations [(NH₂R¹R²)⁺], tertiary ammonium cations [(NH₂R¹R²R³)⁺], quaternary ammonium cations [(NR¹R²R³R⁴)⁺], and combinations thereof, wherein R, R¹, R², R³, and R⁴ are alike or different. In some embodiments, the ammonium salt comprises a cation selected from the group consisting of optionally functionalized tetraalkylammoniums, optionally functionalized tetraarylammoniums, optionally functionalized mixed alkyl-aryl ammoniums, and the like, and combinations thereof. In some embodiments, the ammonium salt comprises a structure selected from the group consisting of [⁺N(R¹)(R²)(R³)(R⁴)]X⁻, [⁺N(R¹)(R²)(R³)(R⁴)]₂X²⁻, and a combination thereof, wherein R¹, R², R³, and R⁴ are alike or different and wherein X represents an anion. In some embodiments, R¹, R², R³, and R⁴ are each independently selected from the group consisting of hydrogen, substituted or unsubstituted optionally functionalized C₁-C₁₀₀ alkyl, substituted or unsubstituted optionally functionalized aryl, and combinations thereof. In some embodiments, two of R¹, R², R³, and R⁴ taken together may optionally form a ring with nitrogen. In some embodiments, covalent bonds may optionally exist between two or more of R¹, R², R³, and R⁴.

In some embodiments, ammonium salts for use in accordance with the present teachings comprise a cation obtained via the protonation of ammonia, primary amines, secondary amines, and/or tertiary amines. In some embodiments, ammonium salts for use in accordance with the present teachings comprise a cation selected from the group consisting of tetraalkyl ammonium cations, tetraaryl ammonium cations, mixed alkyl-aryl ammonium cations, and the like, and combinations thereof. Representative ammonium cations for use in accordance with the present teachings include but are not limited to protonated species obtained by protonation of any primary, secondary, and/or tertiary amine—including but not limited to the amines described herein—as well as tetrasubstituted ammonium salts (e.g., tetraalkyl, tetraaryl, and/or mixed alkyl-aryl), such as tetramethylammonium, tetraethylammonium, tetrapropylammonium, tetraphenylammonium, and the like, and combinations thereof.

In some embodiments, the nitrogen-containing dehydrogenation suppression agent comprises a polyamine. In some embodiments, the polyamine comprises a structure R⁶R⁷N-L-NR⁸R⁹, wherein R⁶, R⁷, R⁸, and R⁹ are alike or different and are each independently selected from the group consisting of hydrogen, substituted or unsubstituted optionally functionalized C₁-C₁₀₀ alkyl, substituted or unsubstituted optionally functionalized aryl, and combinations thereof. In some embodiments, L is a linker selected from the group consisting of (i) substituted or unsubstituted, optionally functionalized aryl groups, (ii) cyclic or acyclic, substituted or unsubstituted, optionally functionalized alkyl groups, and (iii) combinations thereof. Representative polyamines for use in accordance with the present teachings include but are not limited to tetraethylenepentamine, ethylene diamine, 1,3-diaminopropane, 1,2-diaminopropane, 1,2-diaminocyclohexane, 1,3-diaminocyclohexane, 1,4-diaminocyclohexane, 1,4-diaminobutane, 1,3-diaminobutane, 1,2-diaminobutane, N,N-dimethylethylenediamine, N,N′-dimethylethylenediamine, N,N,N′,N′-tetramethylethylenediamine, piperazine, 2-(N,N-diethylamino)ethylamine, N,N-dimethylcyclohexane-1,4-diamine, N,N′-dimethyl-cyclohexane-1,2-diamine, and the like, and combinations thereof.

In some embodiments, the nitrogen-containing dehydrogenation suppression agent comprises nitric acid. It is to be understood that the concentration, origin, purity, physical state, amount of dissolved NO₂, color, and the like of the nitric acid used in accordance with the present teachings is wholly unrestricted, and that all manner of nitric acid is contemplated for use in accordance with these teachings. In some embodiments, the nitric acid is selected from the group consisting of anhydrous nitric acid, fuming nitric acid, concentrated nitric acid, solid hydrates of nitric acid, solutions of nitric acid, and the like, and combinations thereof.

In some embodiments, the dehydrogenation suppression agent comprises anhydrous nitric acid [e.g., about 100 wt % HNO₃ (about 24 M)]. In some embodiments, the dehydrogenation suppression agent comprises fuming nitric acid which, in some embodiments, is selected from the group consisting of strong nitric acid, white fuming nitric acid, red fuming nitric acid, and combinations thereof. In some embodiments, the dehydrogenation suppression agent comprises concentrated nitric acid [e.g., about 68 to about 70 wt % HNO₃ (about 15 to about 16 M)], which, in some embodiments, is selected from the group consisting of technical grade concentrated nitric acid, reagent grade concentrated nitric acid, and a combination thereof. In some embodiments, the dehydrogenation suppression agent comprises mono- or poly-hydrated nitric acid which, in some embodiments, comprises a solid hydrate of nitric acid (e.g. HNO₃.H₂O, HNO₃.3H₂O, etc.). In some embodiments, the dehydrogenation suppression agent comprises a solution of nitric acid. In some embodiments, the solution is aqueous.

In some embodiments, the dehydrogenation suppression agent comprises an aqueous solution of nitric acid in a concentration of between about 0.01 wt % and about 99 wt %. In some embodiments, the concentration is between about 0.1 wt % and about 98 wt %. In some embodiments, the concentration is between about 0.5 wt % and about 90 wt %. In some embodiments, the concentration is between about 1 wt % and about 80 wt %. In some embodiments, the concentration is between about 2 wt % and about 75 wt %. In some embodiments, the concentration is between about 3 wt % and about 70 wt %. In some embodiments, the concentration is between about 4 wt % and about 60 wt %. In some embodiments, the concentration is between about 5 wt % and about 50 wt %. In some embodiments, the concentration is between about 6 wt % and about 40 wt %. In some embodiments, the concentration is between about 5 wt % and about 75 wt %.

In some embodiments, the dehydrogenation suppression agent comprises nitric acid and is attached to a solid support (e.g., silica gel). In some embodiments, the solid support comprises one or more polar functional groups. Representative solid supports for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof.

As presently contemplated, the addition of a dehydrogenation suppression agent to a mixture that comprises (i) olefin metathesis product and/or optionally functionalized olefin reactant, and (ii) metal-containing material in accordance with the present teachings can be practiced whenever it is desirable to prevent dehydrogenation of an olefin metathesis product—particularly though not exclusively cyclic by-products, such as cyclohexadiene—and/or isomerization of an olefin metathesis product—particularly though not exclusively potentially labile olefin products, such as terminal olefins—during any subsequent handling and/or processing including but not limited to heating, distillation, photolytic exposure, exposure to oxidants, and the like, and combinations thereof.

In some embodiments, the dehydrogenation suppression agent is added to a mixture in accordance with the present teachings in a molar excess relative to the metal-containing material. In some embodiments—including but not limited to some embodiments involving phosphorous-containing dehydrogenation suppression agents (e.g., phosphorous acid, phosphinic acid, and the like)—the molar excess is at least about 2 to 1. In some embodiments, the molar excess is at least about 3 to 1. In some embodiments, the molar excess is at least about 4 to 1. In some embodiments, the molar excess is at least about 5 to 1. In some embodiments, the molar excess is at least about 10 to 1. In some embodiments, the molar excess is at least about 15 to 1. In some embodiments, the molar excess is at least about 20 to 1. In some embodiments, the molar excess is at least about 25 to 1. In some embodiments, the molar excess is at least about 30 to 1. In some embodiments, the molar excess is at least about 35 to 1. In some embodiments, the molar excess is at least about 40 to 1. In some embodiments, the molar excess is at least about 45 to 1. In some embodiments, the molar excess is at least about 50 to 1. In some embodiments, the molar excess is at least about 55 to 1. In some embodiments, the molar excess is at least about 60 to 1. In some embodiments, the molar excess is at least about 65 to 1. In some embodiments, the molar excess is at least about 70 to 1. In some embodiments, the molar excess is at least about 75 to 1. In some embodiments, the molar excess is at least about 80 to 1. In some embodiments, the molar excess is at least about 85 to 1. In some embodiments, the molar excess is at least about 90 to 1. In some embodiments, the molar excess is at least about 95 to 1. In some embodiments, the molar excess is at least about 100 to 1.

In some embodiments—including but not limited to some embodiments involving nitric acid-containing dehydrogenation suppression agents—the molar excess the molar excess is less than or equal to about 2 to 1. In some embodiments, the molar excess is less than or equal to about 3 to 1. In some embodiments, the molar excess is less than or equal to about 4 to 1. In some embodiments, the molar excess is less than or equal to about 5 to 1. In some embodiments, the molar excess is less than or equal to about 10 to 1. In some embodiments, the molar excess is less than or equal to about 15 to 1. In some embodiments, the molar excess is less than or equal to about 20 to 1. In some embodiments, the molar excess is less than or equal to about 25 to 1. In some embodiments, the molar excess is less than or equal to about 30 to 1. In some embodiments, the molar excess is less than or equal to about 35 to 1. In some embodiments, the molar excess is less than or equal to about 40 to 1. In some embodiments, the molar excess is less than or equal to about 45 to 1. In some embodiments, the molar excess is less than or equal to about 50 to 1. In some embodiments, the molar excess is less than or equal to about 55 to 1. In some embodiments, the molar excess is less than or equal to about 60 to 1. In some embodiments, the molar excess is less than or equal to about 65 to 1. In some embodiments, the molar excess is less than or equal to about 70 to 1. In some embodiments, the molar excess is less than or equal to about 75 to 1. In some embodiments, the molar excess is less than or equal to about 80 to 1. In some embodiments, the molar excess is less than or equal to about 85 to 1. In some embodiments, the molar excess is less than or equal to about 90 to 1. In some embodiments, the molar excess is less than or equal to about 95 to 1. In some embodiments, the molar excess is less than or equal to about 100 to 1.

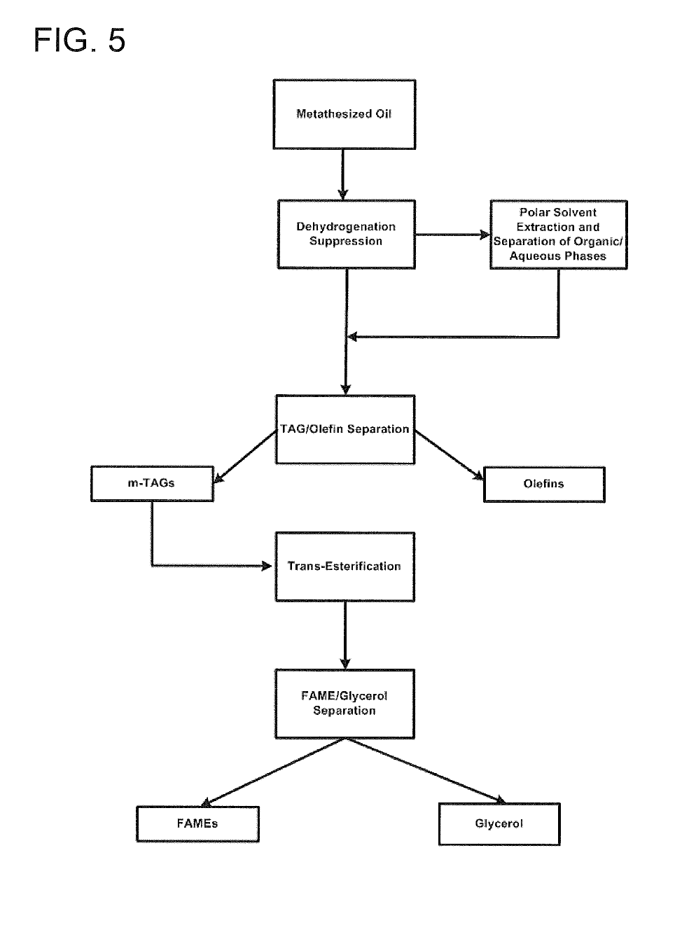

As shown in FIG. 5, in some embodiments, the mixture comprising the olefin metathesis product and/or reactant can be subjected directly to further processing in the presence of the dehydrogenation suppression agent. In other words, in some embodiments, it may not be possible, necessary, and/or desirable to remove the dehydrogenation suppression agent (e.g., via extraction with a polar solvent, such as water) prior to further processing, including but not limited to processing that involves heating. In some embodiments, one such dehydrogenation suppression agent comprises a phosphite ester having a sufficiently high molecular weight and exhibiting a desired degree of thermal stability. In some embodiments, one such dehydrogenation suppression agent comprises a substantially water-insoluble ester of a phosphorous oxo acid which, in some embodiments, may not partition to a significant degree in a polar solvent.

In some embodiments, the dehydrogenation suppression agent is left in the mixture and subjected to further processing but without being thermally stable. In such embodiments, the dehydrogenation suppression agent passivates the metal-containing material before thermally decomposing. One such dehydrogenation suppression agent is THMP which, in some embodiments, appears to undergo thermal decomposition.

In some embodiments, after the dehydrogenation suppression agent has been added to the mixture comprising the (i) olefin metathesis product and/or optionally functionalized olefin reactant, and (ii) metal-containing material, the dehydrogenation suppression agent, in some embodiments, can be left in the mixture and carried along, either in whole or in part, in a subsequent chemical reaction or processing step. In other embodiments, the dehydrogenation suppression agent can optionally be separated and removed from the mixture, either partially or completely, prior to any subsequent reaction or processing step. In some embodiments, passivation and extraction can be coupled into one step (e.g., by providing the dehydrogenation suppression agent in the extracting material).

For embodiments in which it is desirable to separate and/or remove dehydrogenation suppression agent following passivation of a metal-containing material, a method in accordance with the present teachings can optionally further comprise washing or extracting the mixture with a polar solvent (e.g., particularly, though not exclusively, for embodiments in which the dehydrogenation suppression agent is at least partially soluble in the polar solvent). In some embodiments, the polar solvent is at least partially non-miscible with the mixture, such that a separation of layers can occur. In some embodiments, at least a portion of the dehydrogenation suppression agent is partitioned into the polar solvent layer, which can then be separated from the non-miscible remaining layer and removed. Representative polar solvents for use in accordance with the present teachings include but are not limited to water, alcohols (e.g., methanol, ethanol, etc.), ethylene glycol, glycerol, DMF, multifunctional polar compounds including but not limited to polyethylene glycols and/or glymes, ionic liquids, and the like, and combinations thereof. In some embodiments, the mixture is extracted with water. In some embodiments, when a phosphite ester that is at least partially hydrolyzable (e.g., in some embodiments, a phosphite ester having a low molecular weight, including but not limited to trimethyl phosphite, triethyl phosphite, and a combination thereof) is used as a dehydrogenation suppression agent, washing the mixture with water may convert the phosphite ester into a corresponding acid. While neither desiring to be bound by any particular theory nor intending to limit in any measure the scope of the appended claims or their equivalents, it is presently believed that such a hydrolysis may occur more rapidly with lower molecular weight esters.

In some embodiments, the polar solvent used for washing or extracting the metathesis reaction mixture comprises an ionic liquid which, in some embodiments—particularly though not exclusively ones in which the ionic liquid comprises a phosphonium and/or ammonium cation—can further serve as a dehydrogenation suppression agent (thereby allowing passivation of the metal-containing material and washing/extraction to be combined into a single process).

In some embodiments, when extraction with a polar solvent is desired, the extracting may comprise high shear mixing (e.g., mixing of a type sufficient to disperse and/or transport at least a portion of a first phase and/or chemical species into a second phase with which the first phase and/or a chemical species would normally be at least partly immiscible) although such mixing, in some embodiments, may contribute to undesirable emulsion formation. In some embodiments, the extracting comprises low-intensity mixing (e.g., stirring that is not high shear). The present teachings are in no way restricted to any particular type or duration of mixing. However, for purposes of illustration, in some embodiments, the extracting comprises mixing the polar solvent and the mixture together for at least about 1 second. In some embodiments, the extracting comprises mixing the polar solvent and the mixture together for at least about 10 seconds. In some embodiments, the extracting comprises mixing the polar solvent and the mixture together for at least about 30 seconds. In some embodiments, the extracting comprises mixing the polar solvent and the mixture together for at least about 1 minute. In some embodiments, the mixture and the polar solvent are mixed together for at least about 2 minutes, in some embodiments for at least about 5 minutes, in some embodiments for at least about 10 minutes, in some embodiments for at least about 15 minutes, in some embodiments for at least about 20 minutes, in some embodiments for at least about 25 minutes, in some embodiments for at least about 30 minutes, in some embodiments for at least about 35 minutes, in some embodiments for at least about 40 minutes, in some embodiments for at least about 45 minutes, in some embodiments for at least about 50 minutes, in some embodiments for at least about 55 minutes, and in some embodiments for at least about 60 minutes. While neither desiring to be bound by any particular theory nor intending to limit in any measure the scope of the appended claims or their equivalents, it is presently believed that shorter mixing times (e.g., on the order of a second or seconds) are achievable when inline shear mixing is used for mixing.

In some embodiments, the addition of one or more co-solvents can provide a benefit with respect to the requisite mixing time and/or mixing intensity by altering the partition of the dehydrogenation suppression agent in the oil. By way of example, in a process in which the dehydrogenation suppression agent comprises THMP, an alcohol (e.g. isopropyl alcohol) can be added as a co-solvent.

When extraction with a polar solvent is desired, the present teachings are in no way restricted to any particular amount of polar solvent added to the mixture for the extracting. However, for purposes of illustration, in some embodiments, the amount by weight of polar solvent (e.g., water) added to the mixture for the extracting is more than the weight of the mixture. In some embodiments, the amount by weight of polar solvent (e.g., water) added to the mixture for the extracting is less than the weight of the mixture. In some embodiments, the weight ratio of the mixture to the water added to the mixture is at least about 1:1, in some embodiments at least about 2:1, in some embodiments at least about 3:1, in some embodiments at least about 4:1, in some embodiments at least about 5:1, in some embodiments at least about 6:1, in some embodiments at least about 7:1, in some embodiments at least about 8:1, in some embodiments at least about 9:1, in some embodiments at least about 10:1, in some embodiments at least about 20:1, in some embodiments at least about 40:1, and in some embodiments at least about 100:1. For higher oil to water ratios, extraction and separation using a centrifuge and/or coalescer may be desirable.

In some embodiments, when extraction with a polar solvent is desired, methods for suppressing dehydrogenation in accordance with the present teachings further comprise allowing a settling period following the polar solvent wash to promote phase separation. The present teachings are in no way restricted to any particular duration of settling period. However, for purposes of illustration, in some embodiments, the settling period is at least about 1 minute. In some embodiments, the settling period is at least about 2 minutes. In some embodiments, the settling period is at least about 5 minutes. In some embodiments, the settling period is at least about 10 minutes. In some embodiments, the settling period is at least about 15 minutes. In some embodiments, the settling period is at least about 30 minutes. In some embodiments, the settling period is at least about 60 minutes. In some embodiments, the settling period is at least about 120 minutes.

In some embodiments, when extraction with a polar solvent is desired, methods for suppressing dehydrogenation in accordance with the present teachings optionally further comprise separating an organic phase from an aqueous phase, as shown in FIG. 5. In some embodiments, particularly though not exclusively when the dehydrogenation suppression agent is at least partially hydrolysable, a majority of the dehydrogenation suppression agent is distributed in the aqueous phase. In some embodiments, a majority of the olefin metathesis product is distributed in the organic phase. In some embodiments, a majority of the dehydrogenation suppression agent is distributed in the aqueous phase and a majority of the olefin metathesis product is distributed in the organic phase.

In addition to or as an alternative to washing the mixture with a polar solvent to remove dehydrogenation suppression agent—which, in some embodiments, may serve to remove at least a portion of the dehydrogenation suppression agent—a method in accordance with the present teachings can optionally further comprise removing at least a portion of the dehydrogenation suppression agent by adsorbing it onto an adsorbent, which optionally can then be physically separated from the mixture (e.g., via filtration, centrifugation, crystallization, or the like). In some embodiments, the adsorbent is polar. Representative adsorbents for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof.

In some embodiments, the conditions under which a dehydrogenation suppression agent in accordance with the present teachings is added to a mixture that comprises (i) olefin metathesis product and/or optionally functionalized olefin reactant, and (ii) metal-containing material comprise mixing. In some embodiments, the mixing comprises high shear mixing.

In some embodiments, the conditions under which a dehydrogenation suppression agent in accordance with the present teachings is added to a mixture that comprises (i) olefin metathesis product and/or optionally functionalized olefin reactant, and (ii) metal-containing material comprise heating. The present teachings are in no way restricted to any particular heating temperature or range of temperatures. However, for purposes of illustration, in some embodiments, the conditions under which a dehydrogenation suppression agent in accordance with the present teachings is added to a mixture that comprises (i) olefin metathesis product and/or optionally functionalized olefin reactant, and (ii) metal-containing material comprise a heating temperature of about 40° C. or higher. In some embodiments, the heating comprises a temperature of about 50° C. or higher. In some embodiments, the heating comprises a temperature of about 60° C. or higher. In some embodiments, the heating comprises a temperature of about 70° C. or higher. In some embodiments, the heating comprises a temperature of about 80° C. or higher. In some embodiments, the heating comprises a temperature of about 90° C. or higher.

In some embodiments, the molar excess of dehydrogenation suppression agent (relative to catalyst) can affect the residence time required to achieve desired degrees of dehydrogenation and/or olefin isomerization suppression, with higher molar excesses generally corresponding to shorter residence times to achieve comparable degrees of suppression.

The present teachings are in no way restricted to any particular duration of residence time. However, for purposes of illustration, in some embodiments—including but not limited to some embodiments involving phosphorous-containing dehydrogenation suppression agents (e.g., phosphorous acid, phosphinic acid, and the like)—the conditions under which a dehydrogenation suppression agent in accordance with the present teachings is added to a mixture that comprises (i) olefin metathesis product and/or optionally functionalized olefin reactant, and (ii) metal-containing material comprise a residence time of at least about 1 second. In some embodiments, the conditions comprise a residence time of at least about 10 seconds. In some embodiments, the conditions comprise a residence time of at least about 30 seconds. In some embodiments, the conditions comprise a residence time of at least about 1 minute. In some embodiments, the conditions comprise a residence time of at least about 2 minutes. In some embodiments, the conditions comprise a residence time of at least about 3 minutes. In some embodiments, the conditions comprise a residence time of at least about 4 minutes. In some embodiments, the conditions comprise a residence time of at least about 5 minutes. In some embodiments, the conditions comprise a residence time of at least about 10 minutes. In some embodiments, the conditions comprise a residence time of at least about 15 minutes. In some embodiments, the conditions comprise a residence time of at least about 20 minutes. In some embodiments, the conditions comprise a residence time of at least about 25 minutes. In some embodiments, the conditions comprise a residence time of at least about 30 minutes. In some embodiments, the conditions comprise a residence time of at least about 35 minutes. In some embodiments, the conditions comprise a residence time of at least about 40 minutes. In some embodiments, the conditions comprise a residence time of at least about 45 minutes. In some embodiments, the conditions comprise a residence time of at least about 50 minutes. In some embodiments, the conditions comprise a residence time of at least about 55 minutes. In some embodiments, the conditions comprise a residence time of at least about 60 minutes. In some embodiments, the conditions comprise a residence time of one or more hours.

In some embodiments—including but not limited to some embodiments involving nitric acid-containing dehydrogenation suppression agents—the conditions under which a dehydrogenation suppression agent in accordance with the present teachings is added to a mixture that comprises (i) olefin metathesis product and/or optionally functionalized olefin reactant, and (ii) metal-containing material comprise a residence time of less than about 60 minutes. In some embodiments, the residence time is less than about 55 minutes. In some embodiments, the residence time is less than about 50 minutes. In some embodiments, the residence time is less than about 45 minutes. In some embodiments, the residence time is less than about 40 minutes. In some embodiments, the residence time is less than about 35 minutes. In some embodiments, the residence time is less than about 30 minutes. In some embodiments, the residence time is less than about 25 minutes. In some embodiments, the residence time is less than about 20 minutes. In some embodiments, the residence time is less than about 15 minutes. In some embodiments, the residence time is less than about 10 minutes. In some embodiments, the residence time is less than about 5 minutes.

In addition to or as an alternative to facilitating dehydrogenation, the presence of metal-containing material—particularly though not exclusively during heating and/or distillation of an olefin metathesis product and/or reactant—can also result in the isomerization of a carbon-carbon double bond in the product and/or reactant, such that one or more isomers of the original olefin metathesis product and/or reactant are formed. Such isomerization may be undesirable, for example, when end-group functionalization within a product molecule is the goal and isomerization of a desired terminal olefin to an internal olefin is to be avoided. In addition, such isomerization is generally undesirable when it leads to a mixture of products and the goal is a well-defined product in high yield and in high purity. Labile olefins and/or olefins that are not as thermodynamically stable as other isomers readily accessible through isomerization are particularly—though by no means exclusively—susceptible to isomerization (e.g., terminal olefins, vinyl olefins, vinylidene olefins, and the like).

In some embodiments, the olefin metathesis product and/or reactant comprises at least one terminal double bond and, in some embodiments, the isomerization comprises conversion of the terminal double bond to an internal double bond. In some embodiments, the olefin metathesis product and/or reactant comprises at least one internal double bond and, in some embodiments, the isomerization comprises conversion of the internal double bond to a different internal double bond (i.e., an internal double bond between two carbon atoms at least one of which was not part of the original internal double bond). In some embodiments, the olefin metathesis product and/or reactant comprises at least one internal double bond and, in some embodiments, the isomerization comprises conversion of the internal double bond to a terminal double bond. In some embodiments, the suppressing of the isomerization comprises an observed degree of isomerization that is less than about 5%, in some embodiments less than about 4%, in some embodiments less than about 3%, in some embodiments less than about 2%, in some embodiments less than about 1%, in some embodiments less than about 0.9%, in some embodiments less than about 0.8%, in some embodiments less than about 0.7%, in some embodiments less than about 0.6%, in some embodiments less than about 0.5%, in some embodiments less than about 0.4%, in some embodiments less than about 0.3%, in some embodiments less than about 0.2%, and in some embodiments less than about 0.1%.

In some embodiments, methods for suppressing dehydrogenation in accordance with the present teachings can be used in combination with metathesis-based methods for refining natural oil feedstocks. Representative metathesis-based methods for refining natural oil feedstocks include but are not limited to those described in U.S. patent application Ser. No. 12/901,829 (published as United States Patent Application Publication No. 2011/0113679 A1), filed Oct. 11, 2010 (Attorney Docket No. 13687/216), which is assigned to the assignee of the present invention and is incorporated herein by reference in its entirety, except that in the event of any inconsistent disclosure or definition from the present specification, the disclosure or definition herein shall be deemed to prevail.

A number of valuable compositions may be targeted through the self-metathesis reaction of a natural oil feedstock, or the cross-metathesis reaction of the natural oil feedstock with a low-molecular-weight olefin, in the presence of a metathesis catalyst. Such valuable compositions may include but are not limited to fuel compositions, non-limiting examples of which include but are not limited to jet, kerosene, or diesel fuel. Additionally, transesterified products may also be targeted, non-limiting examples of which include but are not limited to: fatty acid methyl esters; biodiesel; 9-decenoic acid (“9DA”) esters, 9-undecenoic acid (“9UDA”) esters, and/or 9-dodecenoic acid (“9DDA”) esters; 9DA, 9UDA, and/or 9DDA; alkali metal salts and alkaline earth metal salts of 9DA, 9UDA, and/or 9DDA; dimers of the transesterified products; and mixtures thereof.

In some embodiments, prior to a metathesis reaction, a natural oil feedstock may be treated to render the natural oil more suitable for the subsequent metathesis reaction. In some embodiments, the natural oil is a vegetable oil or vegetable oil derivative, such as soybean oil.

In some embodiments, the treatment of the natural oil involves the removal of catalyst poisons (i.e., poisons with respect to metathesis activity), such as peroxides, which may potentially diminish the activity of the metathesis catalyst. Non-limiting examples of natural oil feedstock treatment methods to diminish catalyst poisons include but are not limited to those described in WO 2009/020665 A1, WO 2009/020667 A1, and U.S. Patent Application Publication Nos. 2011/0160472 A1 and 2011/0313180 A1. In some embodiments, the natural oil feedstock is thermally treated by heating the feedstock to a temperature greater than 100° C. in the absence of oxygen and held at the temperature for a time sufficient to diminish catalyst poisons in the feedstock. In other embodiments, the temperature is between approximately 100° C. and 300° C., between approximately 120° C. and 250° C., between approximately 150° C. and 210° C., or approximately between 190 and 200° C. In some embodiments, the absence of oxygen is achieved by sparging the natural oil feedstock with a dry, inert gas (including but not limited to nitrogen, argon, and the like, and combinations thereof).

In some embodiments, the natural oil feedstock is chemically treated under conditions sufficient to diminish the catalyst poisons in the feedstock through a chemical reaction of the catalyst poisons. In some embodiments, the feedstock is treated with a reducing agent or a cation-inorganic base composition. Non-limiting examples of reducing agents include but are not limited to bisulfate, borohydride, phosphine, thiosulfate, individually or combinations thereof.

In some embodiments, the natural oil feedstock is treated with an adsorbent to remove catalyst poisons. In some embodiments, the feedstock is treated with a combination of thermal and adsorbent methods. In some embodiments, the feedstock is treated with a combination of chemical and adsorbent methods. In some embodiments, the treatment involves a partial hydrogenation treatment to modify the natural oil feedstock's reactivity with the metathesis catalyst. Additional non-limiting examples of feedstock treatment are also described below when discussing the various metathesis catalysts.

Additionally, in some embodiments, the low-molecular-weight olefin may also be treated prior to the metathesis reaction. Like the natural oil treatment, the low-molecular-weight olefin may be treated to remove poisons that may impact or diminish activity of the catalyst with respect to metathesis.

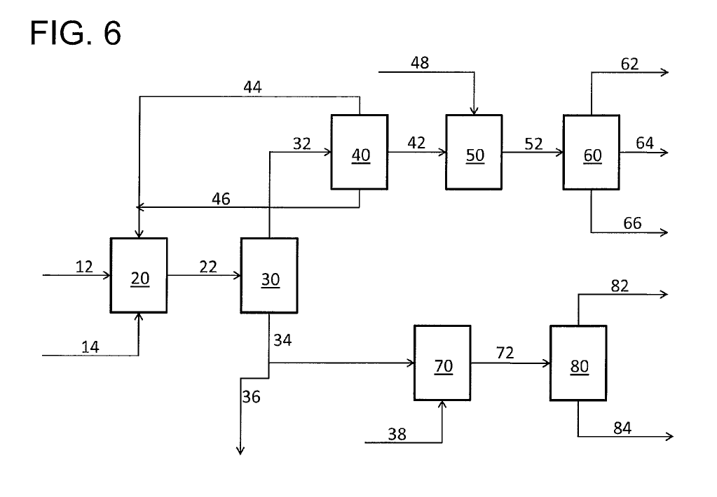

As shown in FIG. 6, after this optional treatment of the natural oil feedstock and/or low-molecular-weight olefin, the natural oil 12 is reacted with itself, or combined with a low-molecular-weight olefin 14 in a metathesis reactor 20 in the presence of a metathesis catalyst. Metathesis catalysts and metathesis reaction conditions are discussed in greater detail below. In some embodiments, in the presence of a metathesis catalyst, the natural oil 12 undergoes a self-metathesis reaction. In other embodiments, in the presence of the metathesis catalyst, the natural oil 12 undergoes a cross-metathesis reaction with the low-molecular-weight olefin 14. In some embodiments, the natural oil 12 undergoes both self- and cross-metathesis reactions in parallel metathesis reactors. The self-metathesis and/or cross-metathesis reaction form a metathesized product 22 wherein the metathesized product 22 comprises olefins 32 and esters 34.

In some embodiments, the low-molecular-weight olefin 14 is in the C₂ to C₆ range. In some embodiments, the low-molecular-weight olefin 14 is selected from the group consisting of ethylene, propylene, 1-butene, 2-butene, isobutene, 1-pentene, 2-pentene, 3-pentene, 2-methyl-1-butene, 2-methyl-2-butene, 3-methyl-1-butene, cyclopentene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, 1,4-pentadiene, 1,4-hexadiene, 1,4-heptadiene, 1,4-octadiene, 1,4-nonadiene, 1,4-decadiene, 2,5-heptadiene, 2,5-octadiene, 2,5-nonadiene, 2,5-decadiene, 3,6-nonadiene, 3,6-decadiene, 1,4,6-octatriene, 1,4,7-octatriene, 1,4,6-nonatriene, 1,4,7-nonatriene, 1,4,6-decatriene, 1,4,7-decatriene, 2,5,8-decatriene, cyclohexene, and the like, and combinations thereof. In some embodiments, the low-molecular-weight olefin 14 comprises at least one of styrene and vinyl cyclohexane. In some embodiments, the low-molecular-weight olefin 14 comprises at least one of ethylene, propylene, 1-butene, 2-butene, and isobutene. In some embodiments, the low-molecular-weight olefin 14 comprises at least one alpha-olefin or terminal olefin in the C₂ to C₁₀ range.

In some embodiments, the low-molecular-weight olefin 14 comprises at least one branched low-molecular-weight olefin in the C₄ to C₁₀ range. Non-limiting examples of branched low-molecular-weight olefins include but are not limited to isobutene, 3-methyl-1-butene, 2-methyl-3-pentene, and 2,2-dimethyl-3-pentene. By using these branched low-molecular-weight olefins in the metathesis reaction, the metathesized product will include but are not limited to branched olefins, which can be subsequently hydrogenated to iso-paraffins. In some embodiments, the branched low-molecular-weight olefins may help achieve the desired performance properties for a fuel composition, such as jet, kerosene, or diesel fuel.

As noted, it is possible to use a mixture of various linear or branched low-molecular-weight olefins in the reaction to achieve the desired metathesis product distribution. In some embodiments, a mixture of butenes (1-butene, 2-butenes, and, optionally, isobutene) may be employed as the low-molecular-weight olefin, offering a low cost, commercially available feedstock instead of a purified source of one particular butene. Such low cost mixed butene feedstocks are typically diluted with n-butane and/or isobutane.

In some embodiments, recycled streams from downstream separation units may be introduced to the metathesis reactor 20 in addition to the natural oil 12 and, in some embodiments, the low-molecular-weight olefin 14. For instance, in some embodiments, a C₂-C₆ recycle olefin stream or a C₃-C₄ bottoms stream from an overhead separation unit may be returned to the metathesis reactor. In some embodiments, as shown in FIG. 6, a light weight olefin stream 44 from an olefin separation unit 40 may be returned to the metathesis reactor 20. In some embodiments, the C₃-C₄ bottoms stream and the light weight olefin stream 44 are combined together and returned to the metathesis reactor 20. In some embodiments, a C₁₅₊ bottoms stream 46 from the olefin separation unit 40 is returned to the metathesis reactor 20. In some embodiments, all of the aforementioned recycle streams are returned to the metathesis reactor 20.

The metathesis reaction in the metathesis reactor 20 produces a metathesized product 22. In some embodiments, the metathesized product 22 enters a flash vessel operated under temperature and pressure conditions which target C₂ or C₂-C₃ compounds to flash off and be removed overhead. The C₂ or C₂-C₃ light ends are comprised of a majority of hydrocarbon compounds having a carbon number of 2 or 3. In some embodiments, the C₂ or C₂-C₃ light ends are then sent to an overhead separation unit, wherein the C₂ or C₂-C₃ compounds are further separated overhead from the heavier compounds that flashed off with the C₂-C₃ compounds. These heavier compounds are typically C₃-C₅ compounds carried overhead with the C₂ or C₂-C₃ compounds. After separation in the overhead separation unit, the overhead C₂ or C₂-C₃ stream may then be used as a fuel source. These hydrocarbons have their own value outside the scope of a fuel composition, and may be used or separated at this stage for other valued compositions and applications. In some embodiments, the bottoms stream from the overhead separation unit containing mostly C₃-C₅ compounds is returned as a recycle stream to the metathesis reactor. In the flash vessel, the metathesized product 22 that does not flash overhead is sent downstream for separation in a separation unit 30, such as a distillation column.

Prior to the separation unit 30, in some embodiments, the metathesized product 22 may be introduced to an adsorbent bed to facilitate the separation of the metathesized product 22 from the metathesis catalyst. In some embodiments, the adsorbent is a clay bed. The clay bed will adsorb the metathesis catalyst, and after a filtration step, the metathesized product 22 can be sent to the separation unit 30 for further processing. In some embodiments, the dehydrogenation suppression agent is a water soluble phosphine reagent (e.g., THMP). Catalyst may be separated with a water soluble phosphine through known liquid-liquid extraction mechanisms by decanting the aqueous phase from the organic phase. In other embodiments, the metathesized product 22 may be contacted with a reactant to deactivate or to extract the catalyst, with a representative reactant being a dehydrogenation suppression agent in accordance with the present teachings.

In the separation unit 30, in some embodiments, the metathesized product 22 is separated into at least two product streams. In some embodiments, the metathesized product 22 is sent to the separation unit 30, or distillation column, to separate the olefins 32 from the esters 34. In some embodiments, a byproduct stream comprising C₇s and cyclohexadiene may be removed in a side-stream from the separation unit 30. In some embodiments, the separated olefins 32 may comprise hydrocarbons with carbon numbers up to C₂₄. In some embodiments, the esters 34 may comprise metathesized glycerides. In other words, the lighter end olefins 32 are preferably separated or distilled overhead for processing into olefin compositions, while the esters 34, comprised mostly of compounds having carboxylic acid/ester functionality, are drawn into a bottoms stream. Based on the quality of the separation, it is possible for some ester compounds to be carried into the overhead olefin stream 32, and it is also possible for some heavier olefin hydrocarbons to be carried into the ester stream 34.

In some embodiments, the olefins 32 may be collected and sold for any number of known uses. In other embodiments, the olefins 32 are further processed in an olefin separation unit 40 and/or hydrogenation unit 50 (where the olefinic bonds are saturated with hydrogen gas 48, as described below). In other embodiments, esters 34 comprising heavier end glycerides and free fatty acids are separated or distilled as a bottoms product for further processing into various products. In some embodiments, further processing may target the production of the following non-limiting examples: fatty acid methyl esters; biodiesel; 9DA esters, 9UDA esters, and/or 9DDA esters; 9DA, 9UDA, and/or 9DDA; alkali metal salts and alkaline earth metal salts of 9DA, 9UDA, and/or 9DDA; diacids, and/or diesters of the transesterified products; and mixtures thereof. In some embodiments, further processing may target the production of C₁₅-C₁₈ fatty acids and/or esters. In other embodiments, further processing may target the production of diacids and/or diesters. In yet other embodiments, further processing may target the production of compounds having molecular weights greater than the molecular weights of stearic acid and/or linolenic acid.

As shown in FIG. 6, regarding the overhead olefins 32 from the separation unit 30, the olefins 32 may be further separated or distilled in the olefin separation unit 40 to separate the stream's various components. In some embodiments, light end olefins 44 consisting of mainly C₂-C₉ compounds may be distilled into an overhead stream from the olefin separation unit 40. In some embodiments, the light end olefins 44 are comprised of a majority of C₃-C₈ hydrocarbon compounds. In other embodiments, heavier olefins having higher carbon numbers may be separated overhead into the light end olefin stream 44 to assist in targeting a specific fuel composition. The light end olefins 44 may be recycled to the metathesis reactor 20, purged from the system for further processing and sold, or a combination of the two. In some embodiments, the light end olefins 44 may be partially purged from the system and partially recycled to the metathesis reactor 20. With regards to the other streams in the olefin separation unit 40, a heavier C₁₆₊, C₁₈₊, C₂₀₊, C₂₂₊, or C₂₄₊ compound stream may be separated out as an olefin bottoms stream 46. This olefin bottoms stream 46 may be purged or recycled to the metathesis reactor 20 for further processing, or a combination of the two. In some embodiments, a center-cut olefin stream 42 may be separated out of the olefin distillation unit for further processing. The center-cut olefins 42 may be designed to target a selected carbon number range for a specific fuel composition. As a non-limiting example, a C₅-C₁₅ distribution may be targeted for further processing into a naphtha-type jet fuel. Alternatively, a C₅-C₁₆ distribution may be targeted for further processing into a kerosene-type jet fuel. In some embodiments, a C₈-C₂₅ distribution may be targeted for further processing into a diesel fuel.

In some embodiments, the olefins 32 may be oligomerized to form poly-alpha-olefins (PAOs) or poly-internal-olefins (PIOs), mineral oil substitutes, and/or biodiesel fuel. The oligomerization reaction may take place after the distillation unit 30 or after the overhead olefin separation unit 40. In some embodiments, byproducts from the oligomerization reactions may be recycled back to the metathesis reactor 20 for further processing.

As mentioned, in some embodiments, the olefins 32 from the separation unit 30 may be sent directly to the hydrogenation unit 50. In some embodiments, the center-cut olefins 42 from the overhead olefin separation unit 40 may be sent to the hydrogenation unit 50. Hydrogenation may be conducted according to any known method in the art for hydrogenating double bond-containing compounds such as the olefins 32 or center-cut olefins 42. In some embodiments, in the hydrogenation unit 50, hydrogen gas 48 is reacted with the olefins 32 or center-cut olefins 42 in the presence of a hydrogenation catalyst to produce a hydrogenated product 52.

In some embodiments, the olefins are hydrogenated in the presence of a hydrogenation catalyst. In some embodiments, the hydrogenation catalyst comprises a metal selected from the group consisting of nickel, copper, palladium, platinum, molybdenum, iron, ruthenium, osmium, rhodium, iridium, and combinations thereof. Useful catalyst may be heterogeneous or homogeneous. In some embodiments, the catalysts are supported nickel or sponge nickel type catalysts.

In some embodiments, the hydrogenation catalyst comprises nickel that has been chemically reduced with hydrogen to an active state (i.e., reduced nickel) provided on a support. The support may comprise porous silica (e.g., kieselguhr, infusorial, diatomaceous, or siliceous earth) or alumina. The catalysts are characterized by a high nickel surface area per gram of nickel.

Commercial examples of supported nickel hydrogenation catalysts include but are not limited to those available under the trade designations “NYSOFACT”, “NYSOSEL”, and “NI 5248 D” (from BASF Catalysts LLC, Iselin, N.J.). Additional supported nickel hydrogenation catalysts include but are not limited to those commercially available under the trade designations “PRICAT 9910”, “PRICAT 9920”, “PRICAT 9908”, “PRICAT 9936” (from Johnson Matthey Catalysts, Ward Hill, Mass.).

The supported nickel catalysts may be of the type described in U.S. Pat. No. 3,351,566, U.S. Pat. No. 6,846,772, EP 0168091, and EP 0167201. Hydrogenation may be carried out in a batch or in a continuous process and may be partial hydrogenation or complete hydrogenation. In some embodiments, the temperature ranges from about 50° C. to about 350° C., about 100° C. to about 300° C., about 150° C. to about 250° C., or about 100° C. to about 150° C. The desired temperature may vary, for example, with hydrogen gas pressure. Typically, a higher gas pressure will require a lower temperature. Hydrogen gas is pumped into the reaction vessel to achieve a desired pressure of H₂ gas. In some embodiments, the H₂ gas pressure ranges from about 15 psig (1 atm) to about 3000 psig (204.1 atm), about 15 psig (1 atm) to about 90 psig (6.1 atm), or about 100 psig (6.8 atm) to about 500 psig (34 atm). As the gas pressure increases, more specialized high-pressure processing equipment may be required. In some embodiments, the reaction conditions are “mild,” wherein the temperature is approximately between approximately 50° C. and approximately 100° C. and the H₂ gas pressure is less than approximately 100 psig. In other embodiments, the temperature is between about 100° C. and about 150° C., and the pressure is between about 100 psig (6.8 atm) and about 500 psig (34 atm). When the desired degree of hydrogenation is reached, the reaction mass is cooled to the desired filtration temperature.

The amount of hydrogenation catalyst is typically selected in view of a number of factors including, for example, the type of hydrogenation catalyst used, the amount of hydrogenation catalyst used, the degree of unsaturation in the material to be hydrogenated, the desired rate of hydrogenation, the desired degree of hydrogenation (e.g., as measure by iodine value (IV)), the purity of the reagent, and the H₂ gas pressure. In some embodiments, the hydrogenation catalyst is used in an amount of about 10 weight % or less, for example, about 5 weight % or less or about 1 weight % or less.

During hydrogenation, the carbon-carbon double bond containing compounds in the olefins are partially to fully saturated by the hydrogen gas 48. In some embodiments, the resulting hydrogenated product 52 includes hydrocarbons with a distribution centered between approximately C₁₀ and C₁₂ hydrocarbons for naphtha- and kerosene-type jet fuel compositions. In some embodiments, the distribution is centered between approximately C₁₆ and C₁₈ for a diesel fuel composition.

In some embodiments, after hydrogenation, the hydrogenation catalyst may be removed from the hydrogenated product 52 using known techniques in the art, for example, by filtration. In some embodiments, the hydrogenation catalyst is removed using a plate and frame filter such as those commercially available from Sparkler Filters, Inc., Conroe Tex. In some embodiments, the filtration is performed with the assistance of pressure or a vacuum. In order to improve filtering performance, a filter aid may be used. A filter aid may be added to the product directly or it may be applied to the filter. Representative non-limiting examples of filtering aids include but are not limited to diatomaceous earth, silica, alumina, and carbon. Typically, the filtering aid is used in an amount of about 10 weight % or less, for example, about 5 weight % or less or about 1 weight % or less. Other filtering techniques and filtering aids also may be employed to remove the used hydrogenation catalyst. In other embodiments the hydrogenation catalyst is removed using centrifugation followed by decantation of the product.

In some embodiments, based upon the quality of the hydrogenated product 52 produced in the hydrogenation unit 50, it may be preferable to isomerize the olefin hydrogenated product 52 to assist in targeting of desired fuel properties such as flash point, freeze point, energy density, cetane number, or end point distillation temperature, among other parameters. Isomerization reactions are well-known in the art, as described in U.S. Pat. Nos. 3,150,205; 4,210,771; 5,095,169; and 6,214,764. In some embodiments, the isomerization reaction at this stage may also crack some of the C₁₅₊ compounds remaining, which may further assist in producing a fuel composition having compounds within the desired carbon number range, such as C₅-C₁₆ for a jet fuel composition.

In some embodiments, the isomerization may occur concurrently with the hydrogenation step in the hydrogenation unit 50, thereby targeting a desired fuel product. In other embodiments, the isomerization step may occur before the hydrogenation step (i.e., the olefins 32 or center-cut olefins 42 may be isomerized before the hydrogenation unit 50). In yet other embodiments, it is possible that the isomerization step may be avoided or reduced in scope based upon the selection of low-molecular-weight olefin(s) 14 used in the metathesis reaction.

In some embodiments, the hydrogenated product 52 comprises approximately 15-25 weight % C₇, approximately <5 weight % C₈, approximately 20-40 weight % C₉, approximately 20-40 weight % C₁₀, approximately <5 weight % C₁₁, approximately 15-25 weight % C₁₂, approximately <5 weight % C₁₃, approximately <5 weight % C₁₄, approximately <5 weight % C₁₅, approximately <1 weight % C₁₆, approximately <1 weight % C₁₇, and approximately <1 weight % C₁₈+. In some embodiments, the hydrogenated product 52 comprises a heat of combustion of at least approximately 40, 41, 42, 43, or 44 MJ/kg (as measured by ASTM D3338). In some embodiments, the hydrogenated product 52 contains less than approximately 1 mg sulfur per kg hydrogenated product (as measured by ASTM D5453). In other embodiments, the hydrogenated product 52 comprises a density of approximately 0.70-0.75 (as measured by ASTM D4052). In other embodiments, the hydrogenated product has a final boiling point of approximately 220-240° C. (as measured by ASTM D86).

The hydrogenated product 52 produced from the hydrogenation unit 50 may be used as a fuel composition, examples of which include but are not limited to jet, kerosene, and diesel fuel. In some embodiments, the hydrogenated product 52 may contain byproducts from the hydrogenation, isomerization, and/or metathesis reactions. As shown in FIG. 6, the hydrogenated product 52 may be further processed in a fuel composition separation unit 60, removing any remaining byproducts from the hydrogenated product 52, such as hydrogen gas, water, C₂-C₉ hydrocarbons, or C₁₅+ hydrocarbons, thereby producing a targeted fuel composition. In some embodiments, the hydrogenated product 52 may be separated into the desired fuel C₉-C₁₅ product 64, and a light-ends C₂-C₉ fraction 62 and/or a C₁₅+ heavy-ends fraction 66. Distillation may be used to separate the fractions. Alternatively, in other embodiments, such as for a naphtha- or kerosene-type jet fuel composition, the heavy ends fraction 66 can be separated from the desired fuel product 64 by cooling the hydrogenated product 52 to approximately −40° C., −47° C., or −65° C. and then removing the solid, heavy ends fraction 66 by techniques known in the art such as filtration, decantation, or centrifugation.

With regard to the esters 34 from the distillation unit 30, in some embodiments, the esters 34 may be entirely withdrawn as an ester product stream 36 and processed further or sold for its own value, as shown in FIG. 6. As a non-limiting example, the esters 34 may comprise various triglycerides that could be used, for example, as a lubricant. Based upon the quality of separation between olefins and esters, the esters 34 may comprise some heavier olefin components carried with the triglycerides. In other embodiments, the esters 34 may be further processed in a biorefinery or another chemical or fuel processing unit known in the art, thereby producing various products such as biodiesel or specialty chemicals that have higher value than that of the triglycerides, for example. Alternatively, in some embodiments, the esters 34 may be partially withdrawn from the system and sold, with the remainder further processed in the biorefinery or another chemical or fuel processing unit known in the art.

In some embodiments, the ester stream 34 is sent to a transesterification unit 70. Within the transesterification unit 70, the esters 34 are reacted with at least one alcohol 38 in the presence of a transesterification catalyst. In some embodiments, the alcohol comprises methanol and/or ethanol. In some embodiments, the transesterification reaction is conducted at approximately 60-70° C. and approximately 1 atm. In some embodiments, the transesterification catalyst is a homogeneous sodium methoxide catalyst. Varying amounts of catalyst may be used in the reaction, and, in some embodiments, the transesterification catalyst is present in the amount of approximately 0.5-1.0 weight % of the esters 34.

The transesterification reaction may produce transesterified products 72 including saturated and/or unsaturated fatty acid methyl esters (“FAME”), glycerin, methanol, and/or free fatty acids. In some embodiments, the transesterified products 72, or a fraction thereof, may comprise a source for biodiesel. In some embodiments, the transesterified products 72 comprise 9DA esters, 9UDA esters, and/or 9DDA esters. Non-limiting examples of 9DA esters, 9UDA esters and 9DDA esters include but are not limited to methyl 9-decenoate (“9-DAME”), methyl 9-undecenoate (“9-UDAME”), and methyl 9-dodecenoate (“9-DDAME”), respectively. As a non-limiting example, in a transesterification reaction, a 9DA moiety of a metathesized glyceride is removed from the glycerol backbone to form a 9DA ester.

In some embodiments, a glycerin alcohol may be used in the reaction with a glyceride stream. This reaction may produce monoglycerides and/or diglycerides.

In some embodiments, the transesterified products 72 from the transesterification unit 70 can be sent to a liquid-liquid separation unit, wherein the transesterified products 72 (i.e., FAME, free fatty acids, and/or alcohols) are separated from glycerin. Additionally, in some embodiments, the glycerin byproduct stream may be further processed in a secondary separation unit, wherein the glycerin is removed and any remaining alcohols are recycled back to the transesterification unit 70 for further processing.

In some embodiments, the transesterified products 72 are further processed in a water-washing unit. In this unit, the transesterified products undergo a liquid-liquid extraction when washed with water. Excess alcohol, water, and glycerin are removed from the transesterified products 72. In some embodiments, the water-washing step is followed by a drying unit in which excess water is further removed from the desired mixture of esters (i.e., specialty chemicals). In some embodiments, a dry wash process can be used in place of water washing. Such specialty chemicals include but are not limited to examples such as 9DA, 9UDA, and/or 9DDA, alkali metal salts and alkaline earth metal salts of the preceding, individually or in combinations thereof.

In some embodiments, the specialty chemical (e.g., 9DA) may be further processed in an oligomerization reaction to form a lactone, which may serve as a precursor to a surfactant.

In some embodiments, the transesterified products 72 from the transesterification unit 70 or specialty chemicals from the water-washing unit or drying unit are sent to an ester distillation column 80 for further separation of various individual or groups of compounds, as shown in FIG. 6. This separation may include but is not limited to the separation of 9DA esters, 9UDA esters, and/or 9DDA esters. In some embodiments, the 9DA ester 82 may be distilled or individually separated from the remaining mixture 84 of transesterified products or specialty chemicals. In certain process conditions, the 9DA ester 82 should be the lightest component in the transesterified product or specialty chemical stream, and come out at the top of the ester distillation column 80. In some embodiments, the remaining mixture 84, or heavier components, of the transesterified products or specialty chemicals may be separated off the bottom end of the column. In some embodiments, this bottoms stream 84 may potentially be sold as biodiesel.

The 9DA esters, 9UDA esters, and/or 9DDA esters may be further processed after the distillation step in the ester distillation column. In some embodiments, under known operating conditions, the 9DA ester, 9UDA ester, and/or 9DDA ester may then undergo a hydrolysis reaction with water to form 9DA, 9UDA, and/or 9DDA, alkali metal salts and alkaline earth metal salts of the preceding, individually or in combinations thereof.

In some embodiments, the fatty acid methyl esters from the transesterified products 72 may be reacted with each other to form other specialty chemicals such as dimers.

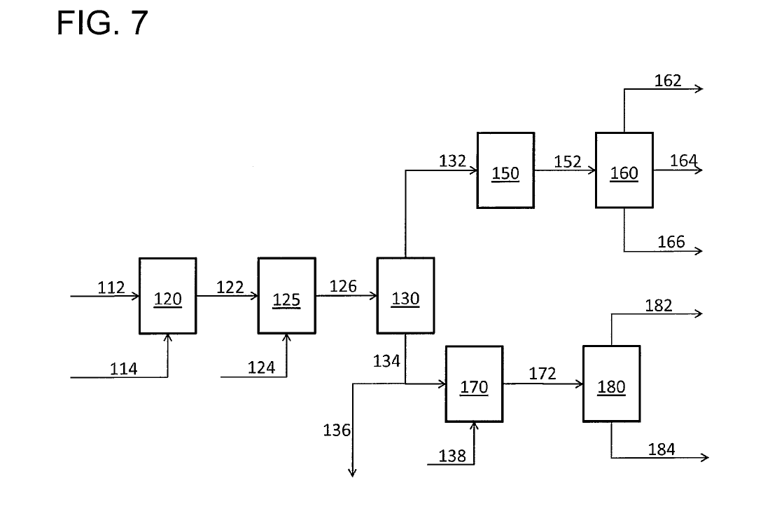

FIG. 7 represents some embodiments for processing the natural oil into fuel compositions and specialty chemicals. As described above, the natural oil feedstock and/or low-molecular-weight olefin in FIG. 7 may undergo a pretreatment step prior to the metathesis reaction. In FIG. 7, the natural oil feedstock 112 is reacted with itself, or combined with a low-molecular-weight olefin 114 in a metathesis reactor 120 in the presence of a metathesis catalyst. In some embodiments, in the presence of a metathesis catalyst, the natural oil 112 undergoes a self-metathesis reaction with itself. In other embodiments, in the presence of the metathesis catalyst, the natural oil 112 undergoes a cross-metathesis reaction with the low-molecular-weight olefin 114. In some embodiments, the natural oil 112 undergoes both self- and/or cross-metathesis reactions in parallel metathesis reactors. The self-metathesis and/or cross-metathesis reaction form a metathesized product 122 wherein the metathesized product 122 comprises olefins 132 and esters 134.

In some embodiments, the low-molecular-weight olefin 114 is in the C₂ to C₆ range. In some embodiments, the low-molecular-weight olefin 114 is selected from the group consisting of ethylene, propylene, 1-butene, 2-butene, isobutene, 1-pentene, 2-pentene, 3-pentene, 2-methyl-1-butene, 2-methyl-2-butene, 3-methyl-1-butene, cyclopentene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, 1,4-pentadiene, 1,4-hexadiene, 1,4-heptadiene, 1,4-octadiene, 1,4-nonadiene, 1,4-decadiene, 2,5-heptadiene, 2,5-octadiene, 2,5-nonadiene, 2,5-decadiene, 3,6-nonadiene, 3,6-decadiene, 1,4,6-octatriene, 1,4,7-octatriene, 1,4,6-nonatriene, 1,4,7-nonatriene, 1,4,6-decatriene, 1,4,7-decatriene, 2,5,8-decatriene, cyclohexene, and the like, and combinations thereof. In some embodiments, the low-molecular-weight olefin 114 comprises at least one of styrene and vinyl cyclohexane. In some embodiments, the low-molecular-weight olefin 114 comprises at least one of ethylene, propylene, 1-butene, 2-butene, and isobutene. In some embodiments, the low-molecular-weight olefin 114 comprises at least one alpha-olefin or terminal olefin in the C₂ to C₁₀ range.

In some embodiments, the low-molecular-weight olefin 114 comprises at least one branched low-molecular-weight olefin in the C₄ to C₁₀ range. Representative examples of branched low-molecular-weight olefins include but are not limited to isobutene, 3-methyl-1-butene, 2-methyl-3-pentene, and 2,2-dimethyl-3-pentene. In some embodiments, the branched low-molecular-weight olefins may help achieve the desired performance properties for the fuel composition, such as jet, kerosene, or diesel fuel.

As noted, it is possible to use a mixture of various linear or branched low-molecular-weight olefins in the reaction to achieve the desired metathesis product distribution. In some embodiments, a mixture of butenes (1-butene, 2-butene, and isobutene) may be employed as the low-molecular-weight olefin 114.

In some embodiments, recycled streams from downstream separation units may be introduced to the metathesis reactor 120 in addition to the natural oil 112 and, in some embodiments, the low-molecular-weight olefin 114 to improve the yield of the targeted fuel composition and/or targeted transesterification products.

After the metathesis unit 120 and before the hydrogenation unit 125, in some embodiments, the metathesized product 122 may be introduced to an adsorbent bed to facilitate the separation of the metathesized product 122 from the metathesis catalyst. In some embodiments, the adsorbent is a clay. The clay will adsorb the metathesis catalyst, and after a filtration step, the metathesized product 122 can be sent to the hydrogenation unit 125 for further processing. In some embodiments, the dehydrogenation suppression agent is a water soluble phosphine reagent (e.g., THMP). Catalyst may be separated from the reaction mixture with a water soluble phosphine through known liquid-liquid extraction mechanisms by decanting the aqueous phase from the organic phase. In other embodiments, addition of a reactant to deactivate or extract the catalyst might be used, with a representative reactant being a dehydrogenation suppression agent in accordance with the present teachings.

As shown in FIG. 7, the metathesis product 122 is sent to a hydrogenation unit 125, wherein the carbon-carbon double bonds in the olefins and esters are partially to fully saturated with hydrogen gas 124. As described above, hydrogenation may be conducted according to any known method in the art for hydrogenating double bond-containing compounds such as the olefins and esters present in the metathesis product 122. In some embodiments, in the hydrogenation unit 125, hydrogen gas 124 is reacted with the metathesis product 122 in the presence of a hydrogenation catalyst to produce a hydrogenated product 126 comprising partially to fully hydrogenated paraffins/olefins and partially to fully hydrogenated esters.

Representative hydrogenation catalysts have been already described with reference to embodiments in FIG. 6. Reaction conditions have also been described. In some embodiments, the temperature ranges from about 50° C. to about 350° C., about 100° C. to about 300° C., about 150° C. to about 250° C., or about 50° C. to about 150° C. The desired temperature may vary, for example, with hydrogen gas pressure. Typically, a higher gas pressure might allow the use of a lower reaction temperature. Hydrogen gas is pumped into the reaction vessel to achieve a desired pressure of H₂ gas. In some embodiments, the H₂ gas pressure ranges from about 15 psig (1 atm) to about 3000 psig (204.1 atm), or about 15 psig (1 atm) to about 500 psig (34 atm). In some embodiments, the reaction conditions are “mild,” wherein the temperature is approximately between approximately 50° C. and approximately 150° C. and the H₂ gas pressure is less than approximately 400 psig. When the desired degree of hydrogenation is reached, the reaction mass is cooled to the desired filtration temperature.

During hydrogenation, the carbon-carbon double bonds are partially to fully saturated by the hydrogen gas 124. In some embodiments, the olefins in the metathesis product 122 are reacted with hydrogen to form a fuel composition comprising only or mostly paraffin. Additionally, the esters from the metathesis product are fully or nearly fully saturated in the hydrogenation unit 125. In some embodiments, the resulting hydrogenated product 126 includes only partially saturated paraffins/olefins and partially saturated esters.

In FIG. 7, the hydrogenated product 126 is sent to a separation unit 130 to separate the product into at least two product streams. In some embodiments, the hydrogenated product 126 is sent to the separation unit 130, or distillation column, to separate the partially to fully saturated paraffins/olefins, or fuel composition 132, from the partially to fully saturated esters 134. In some embodiments, a byproduct stream comprising C₇s, cyclohexadiene, cyclohexene, and/or cyclohexane may be removed in a side-stream from the separation unit 130. In some embodiments, the fuel composition 132 may comprise hydrocarbons with carbon numbers up to C₂₄. In some embodiments, the fuel composition 132 consists essentially of saturated hydrocarbons.

In some embodiments, the esters 134 may comprise metathesized, partially to fully hydrogenated glycerides. In other words, the lighter end paraffins/olefins 132 are preferably separated or distilled overhead for processing into fuel compositions, while the esters 134, comprised mostly of compounds having carboxylic acid/ester functionality are drawn as a bottoms stream. Based on the quality of the separation, it is possible for some ester compounds to be carried into the overhead paraffin/olefin stream 132, and it is also possible for some heavier paraffin/olefin hydrocarbons to be carried into the ester stream 134.

In some embodiments, it may be preferable to isomerize the fuel composition 132 to improve the quality of the product stream and target the desired fuel properties such as flash point, freeze point, energy density, cetane number, or end point distillation temperature, among other parameters. Isomerization reactions are well-known in the art, as described in U.S. Pat. Nos. 3,150,205; 4,210,771; 5,095,169; and 6,214,764. In some embodiments, as shown in FIG. 7, the fuel composition 132 is sent to an isomerization reaction unit 150 wherein an isomerized fuel composition 152 is produced. Under typical reaction conditions, the isomerization reaction at this stage may also crack some of the compounds present in stream 132, which may further assist in producing an improved fuel composition having compounds within the desired carbon number range, such as C₅-C₁₆ for a jet fuel composition.

In some embodiments, the fuel composition 132 or isomerized fuel composition 152 comprises approximately 15-25 weight % C₇, approximately <5 weight % C₈, approximately 20-40 weight % C₉, approximately 20-40 weight % C₁₀, approximately <5 weight % C₁₁, approximately 15-25 weight % C₁₂, approximately <5 weight % C₁₃, approximately <5 weight % C₁₄, approximately <5 weight % C₁₅, approximately <1 weight % C₁₆, approximately <1 weight % C₁₇, and approximately <1 weight % C₁₈+. In some embodiments, the fuel composition 132 or isomerized fuel composition 152 comprises a heat of combustion of at least approximately 40, 41, 42, 43, or 44 MJ/kg (as measured by ASTM D3338). In some embodiments, the fuel composition 132 or isomerized fuel composition 152 contains less than approximately 1 mg sulfur per kg fuel composition (as measured by ASTM D5453). In other embodiments, the fuel composition 132 or isomerized fuel composition 152 comprises a density of approximately 0.70-0.75 (as measured by ASTM D4052). In other embodiments, the fuel composition 132 or isomerized fuel composition 152 has a final boiling point of approximately 220-240° C. (as measured by ASTM D86).

The fuel composition 132 or the isomerized fuel composition 152 may be used as jet, kerosene, or diesel fuel, depending on the fuel's characteristics. In some embodiments, the fuel composition may contain byproducts from the hydrogenation, isomerization, and/or metathesis reactions. The fuel composition 132 or isomerized fuel composition 152 may be further processed in a fuel composition separation unit 160 as shown in FIG. 7. The separation unit 160 may be operated to remove any remaining byproducts from the mixture, such as hydrogen gas, water, C₂-C₉ hydrocarbons, or C₁₅₊ hydrocarbons, thereby producing a desired fuel product 164. In some embodiments, the mixture may be separated into the desired fuel C₉-C₁₅ product 164, and a light-ends C₂-C₉ (or C₃-C₈) fraction 162 and/or a C₁₈+ heavy-ends fraction 166. Distillation, crystallization, and/or other separation techniques may be used to separate the fractions. Alternatively, in other embodiments, such as for a naphtha- or kerosene-type jet fuel composition, the heavy ends fraction 166 can be separated from the desired fuel product 164 by cooling the paraffins/olefins to approximately −40° C., −47° C., or −65° C. and then removing the solid, heavy ends fraction 166 by techniques known in the art such as filtration, decantation, or centrifugation.

With regard to the partially to fully saturated esters 134 from the separation unit 130, in some embodiments, the esters 134 may be entirely withdrawn as a partially to fully hydrogenated ester product stream 136 and processed further or sold for its own value, as shown in FIG. 7. As a non-limiting example, the esters 134 may comprise various partially to fully saturated triglycerides that could be used as a lubricant. Based upon the quality of separation between the paraffins/olefins (fuel composition 132) and the esters, the esters 134 may comprise some heavier paraffin and olefin components carried with the triglycerides. In other embodiments, the esters 134 may be further processed in a biorefinery or another chemical or fuel processing unit known in the art, thereby producing various products such as biodiesel or specialty chemicals that have higher value than that of the triglycerides, for example. Alternatively, the esters 134 may be partially withdrawn from the system and sold, with the remainder further processed in the biorefinery or another chemical or fuel processing unit known in the art.

In some embodiments, the ester stream 134 is sent to a transesterification unit 170. Within the transesterification unit 170, the esters 134 are reacted with at least one alcohol 138 in the presence of a transesterification catalyst. In some embodiments, the alcohol comprises methanol and/or ethanol. In some embodiments, the transesterification reaction is conducted at approximately 60-70° C. and 1 atm. In some embodiments, the transesterification catalyst is a homogeneous sodium methoxide catalyst. Varying amounts of catalyst may be used in the reaction, and, in some embodiments, the transesterification catalyst is present in the amount of approximately 0.5-1.0 weight % of the esters 134.

The transesterification reaction may produce transesterified products 172 including saturated and/or unsaturated fatty acid methyl esters (“FAME”), glycerin, methanol, and/or free fatty acids. In some embodiments, the transesterified products 172, or a fraction thereof, may comprise a source for biodiesel. In some embodiments, the transesterified products 172 comprise decenoic acid esters, decanoic acid esters, undecenoic acid esters, undecanoic acid esters, dodecenoic acid esters, and/or dodecaonic acid esters. In some embodiments, in a transesterification reaction, a decanoic acid moiety of a metathesized glyceride is removed from the glycerol backbone to form a decanoic acid ester. In some embodiments, a decenoic acid moiety of a metathesized glyceride is removed from the glycerol backbone to form a decenoic acid ester.

In some embodiments, a glycerin alcohol may be used in the reaction with a triglyceride stream 134. This reaction may produce monoglycerides and/or diglycerides.

In some embodiments, the transesterified products 172 from the transesterification unit 170 can be sent to a liquid-liquid separation unit, wherein the transesterified products 172 (i.e., FAME, free fatty acids, and/or alcohols) are separated from glycerin. Additionally, in some embodiments, the glycerin byproduct stream may be further processed in a secondary separation unit, wherein the glycerin is removed and any remaining alcohols are recycled back to the transesterification unit 170 for further processing.

In some embodiments, the transesterified products 172 are further processed in a water-washing unit. In this unit, the transesterified products undergo a liquid-liquid extraction when washed with water. Excess alcohol, water, and glycerin are removed from the transesterified products 172. In some embodiments, the water-washing step is followed by a drying unit in which excess water is further removed from the desired mixture of esters (i.e., specialty chemicals). Such hydrogenated specialty chemicals include but are not limited to examples such as decenoic acid, decanoic acid, undecenoic acid, undecanoic acid, dodecenoic acid, dodecanoic acid, and mixtures thereof.

As shown in FIG. 7, the transesterified products 172 from the transesterification unit 170 or specialty chemicals from the water-washing unit or drying unit may be sent to an ester distillation column 180 for further separation of various individual or groups of compounds. This separation may include but is not limited to the separation of decenoic acid esters, decanoic acid esters, undecenoic acid esters, undecanoic acid esters, dodecenoic acid esters, and/or dodecanoic acid esters. In some embodiments, a decanoic acid ester or decenoic acid ester 182 may be distilled or individually separated from the remaining mixture 184 of transesterified products or specialty chemicals. In certain process conditions, the decanoic acid ester or decenoic acid ester 182 should be the lightest component in the transesterified product or specialty chemical stream, and come out at the top of the ester distillation column 180. In some embodiments, the remaining mixture 184, or heavier components, of the transesterified products or specialty chemicals may be separated off the bottom end of the column. In some embodiments, this bottoms stream 184 may potentially be sold as biodiesel.

The decenoic acid esters, decanoic acid esters, undecenoic acid esters, undecanoic acid esters, dodecenoic acid esters, and/or dodecanoic acid esters may be further processed after the distillation step in the ester distillation column. In some embodiments, under known operating conditions, the decenoic acid ester, decanoic acid ester, undecenoic acid ester, undecanoic acid ester, dodecenoic acid ester, and/or dodecanoic acid ester may then undergo a hydrolysis reaction with water to form decenoic acid, decanoic acid, undecenoic acid undecanoic acid, dodecenoic acid, and/or dodecanoic acid.

As noted, the self-metathesis of the natural oil or the cross-metathesis between the natural oil and low-molecular-weight olefin occurs in the presence of a metathesis catalyst. The phrase “metathesis catalyst” includes any catalyst or catalyst system that catalyzes a metathesis reaction. Any known or future-developed metathesis catalyst may be used, individually or in combination with one or more additional catalysts. Non-limiting exemplary metathesis catalysts and process conditions are described in WO 2009/020667 A1 (e.g., pp. 18-47). A number of the metathesis catalysts as shown are manufactured by Materia, Inc. (Pasadena, Calif.).

The metathesis process can be conducted under any conditions adequate to produce the desired metathesis products. For example, stoichiometry, atmosphere, solvent, temperature, and pressure can be selected by one skilled in the art to produce a desired product and to minimize undesirable byproducts. The metathesis process may be conducted under an inert atmosphere. Similarly, if a reagent is supplied as a gas, an inert gaseous diluent can be used. The inert atmosphere or inert gaseous diluent typically is an inert gas, meaning that the gas does not interact with the metathesis catalyst to substantially impede catalysis. For example, particular inert gases are selected from the group consisting of helium, neon, argon, nitrogen, and combinations thereof.

In some embodiments, the metathesis catalyst is dissolved in a solvent prior to conducting the metathesis reaction. In some embodiments, the solvent chosen may be selected to be substantially inert with respect to the metathesis catalyst. For example, substantially inert solvents include but are not limited to, without limitation, aromatic hydrocarbons, such as benzene, toluene, xylenes, etc.; halogenated aromatic hydrocarbons, such as chlorobenzene and dichlorobenzene; aliphatic solvents, including pentane, hexane, heptane, cyclohexane, etc.; and chlorinated alkanes, such as dichloromethane, chloroform, dichloroethane, etc. In some embodiments, the solvent comprises toluene.

In some embodiments, the metathesis catalyst is dissolved in a triglyceride prior to conducting the metathesis reaction. In some embodiments, the triglyceride comprises a saturated, mono-unsaturated, and/or polyunsaturated triglyceride.

The metathesis reaction temperature may be a rate-controlling variable where the temperature is selected to provide a desired product at an acceptable rate. In some embodiments, the metathesis reaction temperature is greater than about −40° C., greater than about −20° C., greater than about 0° C., or greater than about 10° C. In some embodiments, the metathesis reaction temperature is less than about 150° C., or less than about 120° C. In some embodiments, the metathesis reaction temperature is between about 10° C. and about 120° C.

The metathesis reaction can be run under any desired pressure. Typically, it will be desirable to maintain a total pressure that is high enough to keep the cross-metathesis reagent in solution. Therefore, as the molecular weight of the cross-metathesis reagent increases, the lower pressure range typically decreases since the boiling point of the cross-metathesis reagent increases. The total pressure may be selected to be greater than about 0.1 atm (10 kPa), in some embodiments greater than about 0.3 atm (30 kPa), or greater than about 1 atm (100 kPa). Typically, the reaction pressure is no more than about 70 atm (7000 kPa), in some embodiments no more than about 30 atm (3000 kPa). A non-limiting exemplary pressure range for the metathesis reaction is from about 1 atm (100 kPa) to about 30 atm (3000 kPa). In some embodiments, the metathesis reaction can be run under vacuum. By way of example, for self metathesis or cross metathesis of two low vapor pressure reactants, the reaction could be run under reduced pressure (vacuum) to drive away ethylene and other light olefins evolved in process, thereby driving the reaction equilibrium towards higher molecular weight metathesis products (assuming the catalyst remains active).

By way of non-limiting example, in reference to FIG. 6, methods for suppressing dehydrogenation in accordance with the present teachings can be implemented prior to introducing the metathesized product 22 to the separation unit 30 (e.g., a distillation column) and/or at one or more additional stages in the process, including but not limited to prior to initiation of the metathesis reaction (e.g., by introducing a dehydrogenation suppression agent into natural oil 12 and/or low-molecular-weight olefin 14). By way of further non-limiting example, in reference to FIG. 7, methods for suppressing dehydrogenation in accordance with the present teachings can be implemented prior to introducing the metathesized product 122 to the separation unit 130 and/or the hydrogenation unit 125 and/or at one or more additional stages in the process, including but not limited to prior to initiation of the metathesis reaction (e.g., by introducing a dehydrogenation suppression agent into natural oil 112 and/or low-molecular-weight olefin 114). Moreover, in some embodiments—including but not limited to ones in which the dehydrogenation suppression agent is thermally stabile (e.g., a phosphite ester having a sufficiently high molecular weight)—the dehydrogenation suppression agent can be left in the mixture comprising the olefin metathesis product and/or reactant and carried along for further processing (e.g., to the separation units 30 and/or 130 shown, respectively, in FIGS. 6 and 7 and/or to one or more additional units in these or analogous systems). In some embodiments, the dehydrogenation suppression agent is not thermally stabile but is nonetheless left in the mixture comprising the olefin metathesis product and/or reactant and carried along for further processing.

In some embodiments, as shown in FIG. 5, methods for suppressing dehydrogenation of an olefin metathesis product in accordance with the present teachings may optionally further comprise a polar solvent wash—in other words, extracting the mixture to which a dehydrogenation suppression agent has been added with a polar solvent. However, as described above, in some embodiments it may not be possible, necessary, and/or desirable to remove a dehydrogenation suppression agent in accordance with the present teachings via extraction with a polar solvent prior to further processing, which in some embodiments includes but is not limited to processing involving heating.

In some embodiments, the metathesis mixture (e.g., a neat mixture that comprises, in some embodiments, natural oil, metal-containing material, olefin metathesis product, optionally functionalized olefin reactant, and, optionally, low-molecular-weight olefin) is substantially immiscible with the polar solvent, such that two layers are formed. For the sake of convenience, these immiscible layers are described herein as being “aqueous” and “organic” although, in some embodiments, the so-called aqueous layer may be comprised of a polar solvent other than or in addition to water. In some embodiments, the polar solvent extraction (e.g., washing with water) can serve to remove at least a portion of the dehydrogenation suppression agent—particularly though not exclusively when the dehydrogenation suppression agent is at least partially hydrolyzable (e.g., in some embodiments, a phosphite ester having a low molecular weight, including but not limited to trimethyl phosphite, triethyl phosphite, and a combination thereof)—which, in some embodiments, can result in the conversion of a dehydrogenation suppression agent in accordance with the present teachings (e.g., an ester of a phosphorous oxo acid) into a corresponding acid.

In some embodiments—particularly though not exclusively those involving metathesis-based methods for refining natural oil feedstocks—methods for suppressing dehydrogenation in accordance with the present teachings further comprise separating the olefin metathesis product into a metathesized triacylglyceride (m-TAG) fraction and an olefinic fraction, as shown in FIG. 5. In some embodiments, a majority of the triacylglyceride fraction is comprised by molecules comprising one or more carbon-carbon double bonds and, optionally, one or more additional functional groups, whereas a majority of the olefinic fraction is comprised by molecules comprising one or more unsaturated carbon-carbon bonds and no additional functional groups. In some embodiments (e.g., metathesis of palm oil), a majority of the triacylglyceride fraction is comprised by saturated molecules.

In some embodiments—particularly though not exclusively those involving metathesis-based methods for refining natural oil feedstocks—methods for suppressing dehydrogenation in accordance with the present teachings further comprise transesterifying the triacylglyceride fraction to produce one or a plurality of transesterification products, as shown in FIG. 5. In some embodiments, the transesterification products comprise fatty acid methyl esters (FAMEs). In some embodiments—particularly though not exclusively those involving metathesis-based methods for refining natural oil feedstocks—methods for suppressing dehydrogenation in accordance with the present teachings further comprise separating the transesterification products from a glycerol-containing phase, as shown in FIG. 5.

In some embodiments—particularly though not exclusively those involving metathesis-based methods for refining natural oil feedstocks—methods for suppressing dehydrogenation in accordance with the present teachings further comprise separating the olefin metathesis product into a triacylglyceride fraction and an olefinic fraction, transesterifying the triacylglyceride fraction to produce one or a plurality of transesterification products (e.g., FAMEs), and separating the transesterification products from a glycerol-containing phase, as shown in FIG. 5.

In some embodiments, method of refining a natural oil in accordance with the present teachings comprises providing a feedstock comprising a natural oil; reacting the feedstock in the presence of a metathesis catalyst to form a metathesized product comprising olefins and esters; providing a dehydrogenation suppression agent in admixture with the feedstock and/or the metathesized product; passivating a metal-containing material with the dehydrogenation suppression agent; separating the olefins in the metathesized product from the esters in the metathesized product; and transesterifying the esters in the presence of an alcohol to form a transesterified product and/or hydrogenating the olefins to form a fully or partially saturated hydrogenated product. In some embodiments, the metal-containing material comprises residual metathesis catalyst, a hydrogen transfer agent, or a combination thereof. In some embodiments, non-passivated metal-containing material is configured to participate in, catalyze, promote, and/or facilitate dehydrogenation of the natural oil and/or the metathesized product. In some embodiments, the dehydrogenation suppression agent comprises phosphorous. In some embodiments, the dehydrogenation suppression agent comprises nitrogen. In some embodiments, the dehydrogenation suppression agent comprises a quinone, a hydroquinone, or a combination thereof. In some embodiments, the natural oil and/or the metathesized product comprises one or a plurality of substructures having a formula —CH═CH—CH₂—CH═CH—.

In some embodiments, a method of refining a natural oil in accordance with the present teachings further comprises treating the feedstock, prior to reacting the feedstock in the presence of the metathesis catalyst, under conditions sufficient to diminish catalyst poisons in the feedstock. In some embodiments, the feedstock is chemically treated through a chemical reaction to diminish the catalyst poisons. In some embodiments, the feedstock is heated to a temperature greater than 100° C. in an absence of oxygen and held at the temperature for a time sufficient to diminish the catalyst poisons. In some embodiments, the metathesis catalyst is dissolved in a solvent which, in some embodiments, comprises toluene.

In some embodiments, a method of refining a natural oil in accordance with the present teachings further comprises hydrogenating the olefins to form a fuel composition that comprises (a) a jet fuel composition having a carbon number distribution between 5 and 16 and/or (b) a diesel fuel composition having a carbon number distribution between 8 and 25. In some embodiments, the method further comprises oligomerizing the olefins to form a material selected from the group consisting of poly-alpha-olefins, poly-internal-olefins, mineral oil replacements, biodiesel, and the like, and combinations thereof. In some embodiments, the method further comprises separating glycerin from the transesterified product through a liquid-liquid separation, washing the transesterified product with water after separating the glycerin to further remove the glycerin, and drying the transesterified product after the washing to separate the water from the transesterified product.

In some embodiments, the method further comprises distilling the transesterified product to separate a specialty chemical selected from the group consisting of an ester of 9-decenoic acid, an ester of 9-undecenoic acid, an ester of 9-dodecenoic acid, and combinations thereof. In some embodiments, the method further comprises hydrolyzing the specialty chemical, thereby forming an acid selected from the group consisting of 9-decenoic acid, 9-undecenoic acid, 9-dodecenonic acid, alkali metal salts thereof, alkaline metal salts thereof, and combinations thereof.

A first method of producing a fuel composition in accordance with the present teachings comprises providing a feedstock comprising a natural oil; reacting the feedstock in the presence of a metathesis catalyst to form a metathesized product comprising olefins and esters; providing a dehydrogenation suppression agent in admixture with the feedstock and/or the metathesized product; passivating a metal-containing material with the dehydrogenation suppression agent; separating the olefins in the metathesized product from the esters in the metathesized product; and hydrogenating the olefins to form a fuel composition. In some embodiments, the metal-containing material comprises residual metathesis catalyst, a hydrogen transfer agent, or a combination thereof. In some embodiments, non-passivated metal-containing material is configured to participate in, catalyze, promote, and/or facilitate dehydrogenation of the natural oil and/or the metathesized product. In some embodiments, the dehydrogenation suppression agent comprises phosphorous. In some embodiments, the dehydrogenation suppression agent comprises nitrogen. In some embodiments, the dehydrogenation suppression agent comprises a quinone, a hydroquinone, or a combination thereof.

In some embodiments, the fuel composition comprises (a) a kerosene-type jet fuel having a carbon number distribution between 8 and 16, a flash point between about 38° C. and about 66° C., an auto ignition temperature of about 210° C., and a freeze point between about −47° C. and about −40° C.; (b) a naphtha-type jet fuel having a carbon number distribution between 5 and 15, a flash point between about −23° C. and about 0° C., an auto ignition temperature of about 250° C., and a freeze point of about −65° C.; or (c) a diesel fuel having a carbon number distribution between 8 and 25, a specific gravity of between about 0.82 and about 1.08 at about 15.6° C., a cetane number of greater than about 40, and a distillation range between about 180° C. and about 340° C.

In some embodiments, a method of producing a fuel composition in accordance with the present teachings further comprises flash-separating a light end stream from the metathesized product prior to separating the olefins from the esters, the light end stream having a majority of hydrocarbons with carbon number between 2 and 4. In some embodiments, the method further comprises separating a light end stream from the olefins prior to hydrogenating the olefins, the light end stream having a majority of hydrocarbons with carbon numbers between 3 and 8. In some embodiments, the method further comprises separating a C₁₈₊ heavy end stream from the olefins prior to hydrogenating the olefins, the heavy end stream having a majority of hydrocarbons with carbon numbers of at least 18. In some embodiments, the method further comprises separating a C₁₈₊ heavy end stream from the fuel composition, the heavy end stream having a majority of hydrocarbons with carbon numbers of at least 18. In some embodiments, the method further comprises isomerizing the fuel composition, wherein a fraction of normal-paraffin compounds in the fuel composition are isomerized into iso-paraffin compounds.

A second method of producing a fuel composition in accordance with the present teachings comprises providing a feedstock comprising a natural oil; reacting the feedstock in the presence of a metathesis catalyst under conditions sufficient to form a metathesized product comprising olefins and esters; providing a dehydrogenation suppression agent in admixture with the feedstock and/or the metathesized product; passivating a metal-containing material with the dehydrogenation suppression agent; hydrogenating the metathesized product to form a fuel composition and at least partially saturated esters; and separating the fuel composition from the at least partially saturated esters. In some embodiments, the metal-containing material comprises residual metathesis catalyst, a hydrogen transfer agent, or a combination thereof. In some embodiments, non-passivated metal-containing material is configured to participate in, catalyze, promote, and/or facilitate dehydrogenation of the natural oil and/or the metathesized product. In some embodiments, the dehydrogenation suppression agent comprises phosphorous. In some embodiments, the dehydrogenation suppression agent comprises nitrogen. In some embodiments, the dehydrogenation suppression agent comprises a quinone, a hydroquinone, or a combination thereof.

In some embodiments, a method of producing a fuel composition in accordance with the present teachings further comprises isomerizing the fuel composition, such that a fraction of normal-paraffin compounds in the fuel composition are isomerized into iso-paraffin compounds. In some embodiments, the method further comprises separating a C₁₈₊ heavy end stream from the fuel composition, the heavy end stream having a majority of hydrocarbons with carbon numbers of at least 18.

The following examples and representative procedures illustrate features in accordance with the present teachings, and are provided solely by way of illustration. They are not intended to limit the scope of the appended claims or their equivalents.

### EXAMPLES

### Materials and Methods

Unless otherwise indicated, all chemicals were used as received and without drying. Palm oil was obtained from Wilmar International Limited. Soybean oil FAME sold under the tradename Soygold 1100 was purchased from Ag Environmental Products L.L.C. 1-Decene, methyl oleate, toluene, 1,3-cyclohexadiene, n-dodecane, and solid sodium hydroxide, and silica gel (Davisil Grade 633, 60 Å, 200-425 mesh) were purchased from Aldrich. Methyl stearate, methyl palmitate, methyl oleate, methyl linoleate, and methyl linolenate were purchased from Nu-Chek Prep. C827 ruthenium catalyst (Lot No. 01064) was obtained from Materia, Inc. THPS sold under the tradename BRICORR 75 (Lot No. THGS19MI) was obtained from Rhodia Inc. THPS sold under the tradename AQUCAR THPS was obtained from Dow (Lot No. XL25XXXXN1). Deionized water (Type II) was purchased from BDH. Phosphorous acid (spec. 622, neat) and phosphinic acid (spec. 605, 50 wt % in water) were obtained from Special Materials Company. Magnesol Polysorb 30/40 was supplied by Dallas Corporation (SRR 000-60-4).

### Example 1

### General Methodology

A representative and non-limiting process scheme that may be used in accordance with the present teaching is as follows: (a) in a reactor vessel, metathesis catalyst (particularly though not exclusively ruthenium-containing) is mixed with oil containing polyunsaturated fatty acid esters and hydrocarbon olefins under conditions suitable for olefin metathesis; (b) after a desirable conversion of the polyunsaturated fatty acid ester reactants has been achieved, a dehydrogenation suppression agent is added to the reaction mixture to inhibit benzene formation rates; (c) the metathesis product mixture is sent to various unit operations (e.g., liquid-liquid extraction, distillation, crystallization, and/or the like) to separate the products into desirable product streams; and (d) after product separation, the dehydrogenation suppression agent is removed, and some of the product streams are recycled to the process.

As an alternative to (d), in some embodiments—particularly though not exclusively embodiments in which the presence of a dehydrogenation suppression agent does not impede and/or substantially modify the progression of an olefin metathesis reaction—the dehydrogenation suppression agent can be added to the process prior to performing the olefin metathesis reaction, and, after metathesis, removed from the metathesis mixture using liquid-liquid extraction and/or adsorbed onto a material prior to sending the product mixture to various unit operations. While neither desiring to be bound by any particular theory nor intending to limit in any measure the scope of the appended claims or their equivalents, it is presently believed that dehydrogenation suppression agents such as quinones, phosphoesters, and the like, and combinations thereof may be suitable for adding to a reaction mixture prior to performing an metathesis reaction.

An alternative representative and non-limiting process scheme that may be used in accordance with the present teaching is as follows: (a) in a reactor vessel, metathesis catalyst (particularly though not exclusively ruthenium-containing) is mixed with oil containing polyunsaturated fatty acid esters and hydrocarbon olefins under conditions suitable for olefin metathesis; (b) after a desirable conversion of the polyunsaturated fatty acid ester reactants has been achieved, a dehydrogenation suppression agent is added to the reaction mixture to inhibit benzene formation rates (e.g., from 1,4-cyclohexadiene); (c) the metathesis product mixture is sent to various unit operations (e.g., liquid-liquid extraction, adsorption, and/or the like) to remove the dehydrogenation suppression agent and the material capable of participating in, catalyzing and/or otherwise promoting or facilitating a dehydrogenation reaction; (d) the metathesis mixture is sent to various unit operations (e.g. distillation, crystallization, and/or the like) to separate products; (e) after product separation, some of the product streams are recycled to the process.

### Example 2

### Benzene and CHD Formation from Decenolvsis of a Methyl Oleate/Methyl Stearate FAME Mixture

Several experiments to quantify benzene and 1,4-CHD formation rates from the decenolysis of a methyl oleate/methyl stearate FAME mixture are performed under the following targeted conditions:

A FAME mixture with a fatty acid composition containing 9 wt % methyl stearate and 91 wt % methyl oleate) is pretreated at 200° C. for 2 hours with nitrogen sparging. The targeted reactant composition has a 1:1 molar ratio of double bonds in 1-decene to double bonds in FAME mixture. As used herein, the molar ratio of cross agent (e.g., 1-decene) to FAME mixture relates to the molar ratio of double bond content. In the FAME mixture, the double bond content is calculated from the relative ratio of the key fatty acids present (each with its own olefin content), all of which can be readily determined by gas chromatography. Thus, in this example, a 1:1 molar ratio refers to having a 1:1 ratio of cross agent double bonds to the total double bonds of the FAME mixture. The targeted temperature is 60° C. The targeted pressure is 40-100 psig (nitrogen headspace, closed system, temperature dependent). The targeted catalyst loading is 40-80 ppmw catalyst (based on mass of FAME).

The general reaction procedure used is as follows: In a glove box, C827 metathesis catalyst (40.0 mg) is dissolved in toluene (1 mL). The solution (20 pt for experiments 1A and 1B and 40 μL for experiments 2A, 2B, 3A, and 3B in Table 1 below) is sealed into a catalyst addition manifold, removed from a glove box, and attached to a pressure vessel manifold.

The FAME mixture (20 g) at 60° C. is degassed in a pressure vessel for 30 minutes with nitrogen. The outlet to the pressure vessel is closed and catalyst solution is transferred to the vessel using nitrogen. A second pressure vessel containing 1-decene is degassed with nitrogen and pressurized with nitrogen to 100 psig. 1-Decene (11.6 mL) is transferred to the pressure vessel containing FAME through the catalyst addition manifold. The pressure vessel containing the reaction mixture is pressurized to 44-50 psig with nitrogen, and maintained at 60° C. A sample is removed after 120 minutes.

For the experiments summarized in Table 1 below, 1,3-CHD and/or THMP dehydrogenation suppression agent are added as follows: In experiments 1A and 1B, no additional chemicals are added to the reaction mixture. In experiments 2A and 2B, 1,3-CHD is added to the reaction mixture but no dehydrogenation suppression agent is added. In experiment 3A, 1,3-CHD is added into the reaction mixture as in reactions 2A and 2B. In experiment 3B, THMP is added to 3A as a dehydrogenation suppression agent after 2 hours at 60° C.

To accomplish the additions of 1,3-CHD in experiments 2A, 2B and 3A, the catalyst addition manifold is opened under a constant nitrogen purge, and 1,3-CHD (200 μL) is added. The catalyst addition manifold is sealed, and the 1,3-CHD is charged into the vessel using nitrogen pressure. n-Dodecane (200 μL) is added to the catalyst addition manifold as an inert (relative to metathesis activity and isomerization) rinse solvent. The manifold is sealed, and the n-dodecane is transferred into the vessel using nitrogen pressure. The vessel is pressurized to 64 psig through the multiple nitrogen transfers. The reaction is stirred for 15 minutes before another reaction sample is collected. The vessel is re-pressurized to 64 psig using nitrogen after sample is collected.

To accomplish the addition of THMP in experiment 3B, the catalyst addition manifold is opened under constant nitrogen purge, and 1,3-CHD (200 μL) is added. The catalyst addition manifold is sealed, and the 1,3-CHD is charged into the vessel using nitrogen pressure. THMP (60 μL of a solution prepared as described below) is added to the catalyst addition manifold. The manifold is sealed, and THMP is charged into the vessel using nitrogen pressure. n-Dodecane (200 μL) is then used to rinse the catalyst addition manifold in the same fashion. The vessel is pressurized to 70 psig through the multiple nitrogen transfers. The reaction is stirred for 60 minutes before another reaction sample is collected. The vessel is re-pressurized to 70 psig using nitrogen after sample is collected.

The temperature of the reaction vessel is increased to 200° C. and held between 200-210° C. for 60 minutes. After cooling to 60° C. over 60 minutes, a sample is removed.

The neat samples are analyzed within 5 hours of collection for 1,4-CHD and benzene content by GC-FID equipped with an RTX-65TG (Restek). Also, a portion of each sample is diluted with ethyl acetate and analyzed for reactant conversion and product selectivity by GC-FID, using the RTX-65TG described above and an RTX-WAX (Restek).

The general GC analysis procedure is as follows: Retention times for benzene, toluene, 1,4-CHD and 1,3-CHD are identified by authentic samples. Calibrations are created using toluene as an internal standard for all the relevant compounds. Samples of n-dodecane and FAME are injected to verify no contamination or carryover of the identified compounds is present. Toluene concentration is calculated using the density of toluene, the measured mass of the FAME added to the pressure tube, the volume of catalyst solution, and the volume and density of 1-decene transferred to the pressure tube. Additionally, the species are verified by mass, using a separate GC-MS.

A solution of THMP is generated as follows: An aqueous THPS solution (3.3 g, 75 wt % aqueous THPS, Rhodia Inc.) is diluted with a second solution of 5% aqueous sodium hydroxide (4.9 g). The resulting solution is mixed thoroughly, and stored for less than two hours prior to use. Based on P³¹ NMR analysis of similar solutions, the solution from this method primarily contains a mixture of trishydroxymethyl phosphine (THMP ˜−23.5 ppm; phosphoric acid=0 ppm), trishydroxymethyl phosphine oxide (THMPO ˜+49 ppm; phosphoric acid=0 ppm), and tetrakishydroxymethyl phosphonium cation (from THPS ˜+27 ppm; phosphoric acid=0 ppm). For this mixture, the mixture has a distribution of THMP, phosphonium cations from THPS, and THMPO.

Table 1 summarizes the results of benzene formation in the described metathesis reactions.

TABLE 1
Benzene and CHD Formation from Decenolysis of a Methyl Oleate/Methyl
Stearate FAME Mixture

| | Dehydrogenation | | | | | | |
|---|---|---|---|---|---|---|---|
| | Suppression | 1,3- | | Benzene | 1,3- | 1,4- | Benzene/1,3- |
| Expt. | Agent (THMP) | CHD | Sample | Concentration | CHD | CHD | CHD (ppmw/ |
| No. | Added? | Added? | Condition | (ppmw) | (ppmw) | (ppmw) | ppmw) |
| 1A | no | no | 2 h at 60° C. | BDL^{†} | BDL | BDL | NA |
| 1B | no | no | 1 h at 200° C. | BDL | BDL | BDL | NA |
| 2A | no | yes | 2 h at 60° C. | 37 | 6265 | | 0.00590 |
| 2B | no | yes | 1 h at 205° C. | 294 | 3812 | | 0.07714 |
| 3A | no | yes | 2 h at 60° C. | 37 | 7441 | | 0.00491 |
| 3B | yes | yes | 1 h at 205° C. | 56 | 6021 | | 0.00927 |

^{†}BDL = below detectable limits

The data for experiments 1A and 1B in Table 1 indicate that the cross metathesis of a methyl oleate/methyl stearate FAME mixture and 1-decene does not generate 1,3-cyclohexadiene, 1,4-cyclohexadiene, or benzene under experimental conditions. While neither desiring to be bound by any particular theory nor intending to limit in any measure the scope of the appended claims or their equivalents, it is presently believed that this result can be explained by the fact that neither methyl oleate (18:1) nor methyl stearate (18:0) contains at least one —CH═CH—CH₂—CH═CH— substructure that can result in CHD formation by, for example, one of the mechanistic pathways shown in FIGS. 1 and 2.

Moreover, in contrast to the data observed for experiments 1A and 1B in which no benzene forms in the absence of 1,3-CHD, the data for experiments 2A and 2B show when 1,3-CHD is added to the reaction mixture, benzene is formed. Thus, while neither desiring to be bound by any particular theory nor intending to limit in any measure the scope of the appended claims or their equivalents, it is presently believed that 1,4-CHD formed according to the mechanistic pathways shown in FIGS. 1 and 2 may, in the presence of residual metathesis catalyst (or another metal-containing material) initially undergo olefin isomerization to form 1,3-CHD, which can then undergo dehydrogenation to form benzene analogously to the dehydrogenations that are observed in experiments 2A and 2B.

Finally, a comparison of the data from experiments 2B and 3B reveals that the benzene concentration relative to cyclohexadiene concentration is reduced by a factor of about 8 when THMP solution is added to the reaction mixture as a dehydrogenation suppression agent under the conditions studied.

### Example 3

### Benzene Formation from Surrogate Palm Oil FAME Decenolvsis

Several experiments to quantify benzene and 1,4-CHD formation rates from a decenolysis of a surrogate palm FAME mixture (palm FAME) are performed under the following targeted conditions:

A FAME mixture with a fatty acid composition similar to palm oil is pretreated at 200° C. for 2 hours with nitrogen sparging. The palm FAME mixture contains 43.2 wt % methyl palmitate, 4.1 wt % methyl stearate, 41.5 wt % methyl oleate, 10.9 wt % methyl linoleate, and 0.2 wt % methyl linolenate. The targeted reactant composition has a 1:1 molar ratio of double bonds in 1-decene to double bonds in palm FAME mixture. The targeted temperature is 60° C. The targeted pressure is 60 to 110 psig (nitrogen headspace, closed system). The targeted catalyst loading is 40 ppmw (based on mass of palm FAME).

The general reaction procedure used is as follows: In a glove box, C827 metathesis catalyst (40.0 mg) is dissolved in toluene (1 mL). The solution (20 μL) is sealed into a catalyst addition manifold, removed from the glove box, and attached to a pressure vessel manifold.

The surrogate palm FAME (20 g) at 60° C. is degassed in a pressure vessel for 30 minutes with nitrogen. The outlet to the pressure vessel is closed and catalyst solution is transferred to the pressure vessel using nitrogen. A second pressure vessel containing 1-decene is degassed with nitrogen and pressured with nitrogen to 100 psig. 1-Decene (8.23 mL) is transferred to the pressure vessel containing surrogate palm FAME through the catalyst addition manifold, and residual catalyst solution is washed into the vessel. The pressure vessel containing the reaction mixture is pressurized to 60 psig with nitrogen. A sample is removed after 120 minutes.

For the experiments summarized in Table 2 below, dehydrogenation suppression agents are added as follows: In experiments 4A, 4B, 5A, 5B 6A, and 7A, no additional chemicals are added. In experiment 6B, THMP is added. In experiments 7B, THPS is added.

To accomplish the addition of dehydrogenation suppression agent in experiment 6B, the catalyst addition manifold is opened under constant nitrogen purge, and THMP (50 μL of an aqueous solution, prepared as described below) is added. The catalyst addition manifold is sealed, and THMP is charged into the vessel using nitrogen pressure. The manifold is again opened under constant nitrogen purge, and rinsed with deionized water (150 μL) in the same manner. The reaction is stirred for 30 minutes before another reaction sample is collected.

To accomplish the addition of dehydrogenation suppression agent in experiment 7B, the sample loop is opened under constant nitrogen purge, and THPS (10 μL, 75 wt % aqueous THPS, Dow) is added. The catalyst addition manifold is sealed, and THPS is charged into the vessel using nitrogen pressure. The manifold is again opened under constant nitrogen purge, and rinsed with deionized water (3×50 4) in the same manner. The reaction is stirred for 30 minutes before another reaction sample is collected.

The temperature of the reaction vessel is increased to 225° C. over 45 minutes and held between 225-230° C. for 60 minutes. After cooling to 65° C. over 60 minutes, a sample is removed.

The neat samples are analyzed for 1,4-CHD and benzene content within 5 hours of collection by GCMS equipped with a quadrupole mass spectrometer using an RTX-65TG (Restek). Also, a portion of each sample is diluted with ethyl acetate and analyzed for reactant conversion and product selectivity by GCFID using a RTX-WAX (Restek).

The general GCMS analysis procedure is as follows: Retention times for benzene and 1,4-CHD are identified by authentic samples. Calibration curves are created using toluene as an internal standard for relevant compounds. Samples of n-dodecane and palm FAME are injected to verify no contamination or carryover of the identified compounds is present. The ion fragmentation patterns are extracted using the main ion fragment in a window containing the anticipated retention times. Toluene concentration is calculated using the density of toluene, the measured mass of the surrogate palm FAME added to the pressure tube, the volume of catalyst solution, and the volume and density of 1-decene transferred to the pressure tube.

A THPS solution is generated as follows: An aqueous THPS solution (10.1 g, 75 wt % aqueous THPS, Dow) is diluted with deionized water (28.0 g). The mixture is stirred and degassed with nitrogen for 30 minutes. A pH of 3.17 is measured. The THPS solution is treated with a solution of 50% aqueous sodium hydroxide (1.6 g) and mixed for 15 minutes. A pH of 7.27 is measured. Additional sodium hydroxide solution (1.32 g) is added and mixed for 15 minutes. A pH of 11.72 is measured. After an additional 30 minutes of mixing, a pH of 11.44 is measured. Based on P³¹ NMR of this solution, the solution primarily contains a mixture of trishydroxymethyl phosphine (THMP ˜−23.5 ppm; phosphoric acid=0 ppm), trishydroxymethyl phosphine oxide (THMPO ˜+49 ppm; phosphoric acid=0 ppm), and tetrakishydroxymethyl phosphonium cation (from THPS ˜+27 ppm; phosphoric acid=0 ppm). For this mixture, the predominant P-containing species in the mixture is THMPO.

Table 2 below summarizes the results of benzene formation in the described metathesis reactions.

TABLE 2
Benzene and CHD Formation from Decenolysis of a
Surrogate Palm FAME Mixture

| | Dehydro- | | | | |
|---|---|---|---|---|---|
| | genation | | | | |
| | Suppression | | Benzene | | Benzene/ |
| | Agent | | Concen- | 1,3- | 1,3-CHD |
| Expt. | (THMP) | Sample | tration | CHD | (ppmw/ |
| No. | Added? | Condition | (ppmw) | (ppmw) | ppmw) |
| 4A | no | 2 h at 60° C. | 0.34 | 2083 | 0.00016 |
| 4B | no | 1 h at 225° C. | 31.51 | 2470 | 0.01276 |
| 5A | no | 2 h at 60° C. | 0.97 | 2817 | 0.00034 |
| 5B | no | 1 h at 225° C. | 32.86 | 3955 | 0.00831 |
| 6A | no | 2 h at 60° C. | BDL^{†} | 1506 | 0.00000 |
| 6B | yes | 1 h at 225° C. | 4.96 | 1695 | 0.00293 |
| 7A | no | 2 h at 60° C. | BDL | 1716 | 0.00000 |
| 7B | yes | 1 h at 225° C. | 5.92 | 1728 | 0.00343 |

^{†}BDL = below detectable limits (<0.1)

The data shown in Table 2 indicate that the benzene concentration relative to cyclohexadiene concentration is reduced by a factor of at least 2 when adding THMP or THPS solutions to the reaction mixture under the conditions studied.

### Example 4

### Benzene and CHD Formation from Decenolysis of Soybean Oil FAME

Several experiments to quantify benzene and 1,4-CHD formation rates from the decenolysis of a soybean oil FAME mixture (soy FAME) are performed under the following targeted conditions:

Soygold 1100 is pretreated at 200° C. for 2 hours with nitrogen sparging. The targeted reactant composition has a 1:1 molar ratio of double bonds in 1-decene to double bonds in soy FAME mixture. The targeted temperature is 60° C. The targeted pressure is 70-132 psig (nitrogen headspace, closed system). The targeted catalyst loading is 80 ppmw (based on mass of soy FAME).

The general reaction procedure used is as follows: In a glove box, C827 metathesis catalyst (40.0 mg) is dissolved in toluene (1 mL). The solution (40 μL) is sealed into a catalyst addition manifold, removed from the glove box, and attached to a pressure vessel manifold.

Soy FAME (20.0 g) at 60° C. is degassed in a pressure vessel for 30 minutes with nitrogen. The outlet to the pressure vessel is closed and catalyst solution is transferred to the vessel using nitrogen. A second pressure vessel containing 1-decene is degassed with nitrogen and pressurized with nitrogen to 100 psig. 1-Decene (19.9 mL) is transferred to the pressure vessel containing soy FAME through the catalyst addition manifold, and residual catalyst solution is washed into the vessel. The pressure vessel containing the reaction mixture is pressurized to 70 psig with nitrogen. A sample is removed after 120 minutes.

For the experiments summarized in Table 3 below, dehydrogenation suppression agent is added as follows: In experiments 8A, 8B, 9A, and 10A, no additional chemicals are added. In experiments 9B and 9C, THMP dehydrogenation suppression agent is added. In experiments 10B and 10C, phosphorous acid dehydrogenation suppression agent is added.

To accomplish the addition of dehydrogenation suppression agent in experiments 9B and 9C, the catalyst addition manifold is opened under a constant nitrogen purge, and THMP (60 μL of an aqueous solution, prepared as described below) is added. The catalyst addition manifold is sealed, and the THMP is charged into the vessel using nitrogen pressure. The manifold is again opened under constant nitrogen purge, and rinsed with deionized water (100 μL) in the same manner. This process is repeated two additional times to give a total of 300 μL. The reaction is stirred for 60 minutes before another reaction sample is collected.

To accomplish the addition of dehydrogenation suppression agent in experiments 10B and 10C, the sample loop is opened under constant nitrogen purge, and phosphorous acid (80 μL of a 10 wt % aqueous solution, prepared as described below) is added. The catalyst addition manifold is sealed, and the phosphorous acid is charged into the vessel using nitrogen pressure. The manifold is again opened under constant nitrogen purge, and rinsed with deionized water (100 pt) in the same manner. This process is repeated two additional times to give a total of 300 μL. The reaction is stirred for 60 minutes before another reaction sample is collected.

The temperature of the reaction vessel is increased to 225° C. over 45 minutes and held between 225-230° C. for 60 minutes. After cooling to 60° C. over 60 minutes, a sample is removed.

The neat samples are analyzed for 1,4-CHD and benzene content within 5 hours of collection by GC-FID equipped with an RTX-65TG (Restek). Also, a portion of each sample is diluted with ethyl acetate and analyzed for reactant conversion and product selectivity by GC-FID using the RTX-65TG above and an RTX-WAX (Restek).

The general GC analysis procedure is as follows: Retention times for benzene, toluene and 1,4-CHD are identified by authentic samples. Calibration curves are created using toluene as an internal standard for relevant compounds. Samples of n-dodecane and soy FAME are injected to verify no contamination or carryover of the identified compounds is present. Gas chromatographs are analyzed for benzene, 1,4-CHD, and toluene. Toluene concentration is calculated using the density of toluene, the measured mass of the soy FAME added to the pressure tube, the volume of catalyst solution, and the volume and density of 1-decene transferred to the pressure tube. Additionally, the species are verified by mass, using a separate GC-MS.

A solution of THMP is generated as follows: An aqueous THPS solution (4.0 g, target, 75 wt %) is diluted with a second solution of 5% aqueous sodium hydroxide (5.9 g). The resulting solution is mixed thoroughly, and stored for less than two hours prior to use. Based on P³¹ NMR analysis of similar solutions, the solution from this method primarily contains a mixture of trishydroxymethyl phosphine (THMP ˜−23.5 ppm; phosphoric acid=0 ppm), trishydroxymethyl phosphine oxide (THMPO ˜+49 ppm; phosphoric acid=0 ppm), and tetrakishydroxymethyl phosphonium cation (from THPS ˜+27 ppm; phosphoric acid=0 ppm). For this mixture, the mixture has a distribution of THMP, phosphonium cations from THPS, and THMPO. A 10 wt % phosphorous acid solution is prepared by diluting solid phosphorous acid (1.0 g) with deionized water (9.0 g).

Table 3 summarizes the results of benzene formation in the described metathesis reactions.

TABLE 3
Benzene and CHD Formation from Decenolysis
of a Soy FAME Mixture

| | Dehydro- | | | Cyclo- | Benzene/ |
|---|---|---|---|---|---|
| | genation | | Benzene | hexadiene | Cyclo- |
| | Suppression | | Concen- | Concen- | hexadiene |
| Expt. | Agent | Sample | tration | tration | (ppmw/ |
| No. | Added? | Condition | (ppmw) | (ppmw) | ppmw) |
| 8A | no | 2 h at 60° C. | 14 | 5808 | 0.00241 |
| 8B | no | 1 h at 225° C. | 184 | 5689 | 0.03234 |
| 9A | no | 2 h at 60° C. | 16 | 7117 | 0.00225 |
| 9B | THMP | 1 h at 60° C. | 19 | 7665 | 0.00248 |
| 9C | THMP | 1 h at 225° C. | 19 | 7909 | 0.00240 |
| 10A | no | 2 h at 60° C. | 17 | 7563 | 0.00225 |
| 10B | phosphorous | 1 h at 60° C. | 20 | 8814 | 0.00227 |
| | acid | | | | |
| 10C | phosphorous | 1 h at 225° C. | 17 | 8633 | 0.00197 |
| | acid | | | | |

The data in Table 3 indicate that the benzene concentration relative to cyclohexadiene concentration is reduced by a factor of about 13 when adding THMP or phosphorous acid solutions to the reaction mixture under the conditions studied.

### Example 5

### Benzene and CHD Formation from Decenolysis of a Soybean Oil FAME

Several experiments to quantify benzene and 1,4-CHD formation rates from the decenolysis of a soybean oil FAME mixture (soy FAME) are performed under the following targeted conditions:

Soygold 1100 is pretreated at 200° C. for 2 hours with nitrogen sparging. The targeted reactant composition comprises a 1:1 molar ratio of double bonds in 1-decene to double bonds in soy FAME mixture. The targeted temperature is 60° C. The targeted pressure is 70-132 psig (nitrogen headspace, closed system, temperature dependent). The targeted catalyst loading is 80 ppmw catalyst (based on mass of soy FAME).

The general reaction procedure applied is as follows: In a glove box, C827 metathesis catalyst (40.0 mg) is dissolved in toluene (1 mL). The solution (40 μL) is sealed into a catalyst addition manifold, removed from a glove box, and attached to a pressure vessel manifold.

Soy FAME (20 g) at 60° C. is degassed in a pressure vessel for 30 minutes with nitrogen. The outlet to the pressure vessel is closed and catalyst solution is transferred to the vessel using nitrogen. A second pressure vessel containing 1-decene is degassed with nitrogen and is pressurized with nitrogen to 100 psig. 1-Decene (19.9 mL) is transferred to the pressure vessel containing soy FAME through the catalyst addition manifold, and residual catalyst solution is washed into the vessel. The pressure vessel containing the reaction mixture is pressurized to 70 psig with nitrogen. A sample is removed after 120 minutes.

Dehydrogenation suppression agent is added as follows: In experiment 11A, phosphinic acid dehydrogenation suppression agent is added. In experiment 11B, diethyl phosphite dehydrogenation suppression agent is added. In experiment 110, 2-amino-2-hydroxymethyl-propane-1,3-diol dehydrogenation suppression agent is added. In experiment 11D, a tetraethylammonium sulfate dehydrogenation suppression agent is added. In experiment 11E, nitric acid dehydrogenation suppression agent is added. In experiment 11F, trishydroxymethyl phosphine oxide dehydrogenation suppression agent is added. In experiment 11G, trishydroxymethyl phosphine dehydrogenation suppression agent is added.

To accomplish the addition of dehydrogenation suppression agent in experiments, the catalyst addition manifold is opened under a constant nitrogen purge, and the dehydrogenation suppression agent is added at a 50:1 molar equivalent ratio of dehydrogenation suppression agent to catalyst in the mixture; the suppression agent is added as a 10 mass % aqueous solution. The catalyst addition manifold is sealed, and the suppression agent is charged into the vessel using nitrogen pressure. The manifold is again opened under constant nitrogen purge, and is rinsed with deionized water (100 μL) in the same manner. This process is repeated two additional times to give a total of 300 μL. The reaction is stirred for 60 minutes and another reaction sample is collected.

The temperature of the reaction vessel is increased to 225° C. over 45 minutes, is held between 225-230° C. for 60 minutes, and is cooled to 60° C. over 60 minutes. When the mixture reaches 60° C., a sample is removed.

### Example 6

### Benzene and CHD Formation from Decenolysis of a Soybean Oil FAME

Several experiments to quantify benzene and 1,4-CHD formation rates from the decenolysis of a soybean oil FAME mixture (soy FAME) are performed under the following targeted conditions:

Soygold 1100 is pretreated at 200° C. for 2 hours with nitrogen sparging. The targeted reactant composition comprises a 1:1 molar ratio of double bonds in 1-decene to double bonds in soy FAME mixture. The targeted temperature is 60° C. The targeted pressure is 70-132 psig (nitrogen headspace, closed system, temperature dependent). The targeted catalyst loading is 80 ppmw catalyst (based on mass of soy FAME).

The general reaction procedure applied is as follows: In a glove box, C827 metathesis catalyst (40.0 mg) is dissolved in toluene (1 mL). The solution (40 μL) is sealed into a catalyst addition manifold, removed from a glove box, and attached to a pressure vessel manifold.

Soy FAME (20 g) at 60° C. is degassed in a pressure vessel for 30 minutes with nitrogen. The outlet to the pressure vessel is closed, and the catalyst solution is transferred to the vessel using nitrogen. A second pressure vessel containing 1-decene is degassed with nitrogen and is pressurized with nitrogen to 100 psig. 1-Decene (19.9 mL) is transferred to the pressure vessel containing soy FAME through the catalyst addition manifold, and residual catalyst solution is washed into the vessel. The pressure vessel containing the reaction mixture is pressurized to 70 psig with nitrogen. A sample is removed after 120 minutes.

Dehydrogenation suppression agent is added as follows: In experiment 12A, phosphinic acid dehydrogenation suppression agent is added. In experiment 12B, diethyl phosphite dehydrogenation suppression agent is added. In experiment 12C, 2-amino-2-hydroxymethyl-propane-1,3-diol dehydrogenation suppression agent is added. In experiment 12D, a tetraethylammonium sulfate dehydrogenation suppression agent is added. In experiment 12E, nitric acid dehydrogenation suppression agent is added. In experiment 12F, trishydroxymethyl phosphine oxide dehydrogenation suppression agent is added. In experiment 12G, trishydroxymethyl phosphine dehydrogenation suppression agent is added.

To accomplish the addition of dehydrogenation suppression agent in experiments, the catalyst addition manifold is opened under a constant nitrogen purge, and the dehydrogenation suppression agent is added as a 10 mass % aqueous solution at a 50:1 molar equivalent ratio of dehydrogenation suppression agent to catalyst in the mixture. The catalyst addition manifold is sealed, and the suppression agent is charged into the vessel using nitrogen pressure. The manifold is again opened under constant nitrogen purge, and is rinsed with deionized water (100 μL) in the same manner. This process is repeated two additional times to give a total of 300 μL. The reaction is stirred for 60 minutes and another reaction sample is collected.

Water is added to the vessel at a ratio of 1 g of water to 5 g of metathesized oil mixture. The water-oil mixture is heated to 90° C. and is stirred for 60 minutes. The stirring is stopped, and the mixture settles under gravity for 60 minutes at 90° C. into two phases, a water phase (upper layer) and an organic oil phase (lower layer). The water phase is removed from the vessel.

The temperature of the reaction vessel is increased to 225° C. over 45 minutes, is held between 225-230° C. for 60 minutes, and is cooled to 60° C. over 60 minutes. When the mixture reaches 60° C., a sample is removed.

### Example 7

### Benzene and CHD Formation from Decenolvsis of a Soybean Oil FAME

Several experiments to quantify benzene and 1,4-CHD formation rates from the decenolysis of a soybean oil FAME mixture (soy FAME) are performed under the following targeted conditions:

Soygold 1100 is pretreated at 200° C. for 2 hours with nitrogen sparging. The targeted reactant composition comprises a 1:1 molar ratio of double bonds in 1-decene to double bonds in soy FAME mixture. The targeted temperature is 60° C. The targeted pressure is 70-132 psig (nitrogen headspace, closed system, temperature dependent). The targeted catalyst loading is 80 ppmw catalyst (based on mass of soy FAME).

The general reaction procedure applied is as follows: In a glove box, C827 metathesis catalyst (40.0 mg) is dissolved in toluene (1 mL). The solution (40 μL) is sealed into a catalyst addition manifold, removed from the glove box, and attached to a pressure vessel manifold.

Soy FAME (20 g) and dehydrogenation suppression agent are charged to the pressure vessel. Dehydrogenation suppression agent is added as follows: In experiment 13A, 1,4-benzoquinone dehydrogenation suppression agent is added. In experiment 13B, monophenyl phosphoester dehydrogenation suppression agent is added to reaction pressure vessel. The dehydrogenation suppression agent is added as a neat material at a 0.5 mass %, based on mass of oil in the mixture. The mixture is heated to 60° C. and is degassed for 30 minutes with nitrogen.

The outlet to the pressure vessel is closed and catalyst solution is transferred to the vessel using nitrogen. A second pressure vessel containing 1-decene is degassed with nitrogen and is pressurized with nitrogen to 100 psig. 1-Decene (19.9 mL) is transferred to the pressure vessel containing soy FAME through the catalyst addition manifold, and residual catalyst solution is washed into the vessel. The pressure vessel containing the reaction mixture is pressurized to 70 psig with nitrogen. A sample is removed after 120 minutes through pressurized nitrogen transfer.

The temperature of the reaction vessel is increased to 225° C. over 45 minutes, is held between 225-230° C. for 60 minutes, and is cooled to 60° C. over 60 minutes. When the mixture reaches 60° C., a sample is removed through pressurized nitrogen transfer.

The entire contents of every document cited hereinabove are hereby incorporated by reference, except that in the event of any inconsistent disclosure or definition from the present specification, the disclosure or definition herein shall be deemed to prevail.

In addition, each of the following patent applications—assigned to the assignee of the present invention—is also incorporated herein by reference in its entirety, except that in the event of any inconsistent disclosure or definition from the present specification, the disclosure or definition herein shall be deemed to prevail: U.S. patent application Ser. No. 13/335,466 filed Dec. 22, 2011 (Attorney Docket No. 13687/283; ERS-004); U.S. patent application Ser. No. 13/335,495 filed Dec. 22, 2011 (Attorney Docket No. 13687/284; ERS-005); U.S. patent application Ser. No. 13/335,517 filed Dec. 22, 2011 (Attorney Docket No. 13687/285; ERS-006); U.S. patent application Ser. No. 13/335,538 filed Dec. 22, 2011 (Attorney Docket No. 13687/296; ERS-009); U.S. patent application Ser. No. 13/335,584 filed Dec. 22, 2011 (Attorney Docket No. 13687/297; ERS-010); U.S. patent application Ser. No. 13/335,601 filed Dec. 22, 2011 (Attorney Docket No. 13687/298; ERS-011); and U.S. provisional patent application Ser. No. 61/250,743, filed Oct. 12, 2009 (Attorney Docket No. 13687/145).

The foregoing detailed description and accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. A method for suppressing dehydrogenation comprising:
reacting an optionally functionalized olefin reactant in a metathesis reaction to form an olefin metathesis product; and
providing a dehydrogenation suppression agent in admixture with (a) the olefin metathesis product and/or the optionally functionalized olefin reactant, and (b) a metal-containing material selected from the group consisting of residual metathesis catalyst, a hydrogen transfer agent, and a combination thereof, under conditions that are sufficient to passivate at least a portion of the metal-containing material;
wherein non-passivated metal-containing material is configured to participate in, catalyze, promote, and/or facilitate dehydrogenation of the optionally functionalized olefin reactant and/or the olefin metathesis product.

2. The invention of claim 1 wherein the optionally functionalized olefin reactant and/or the olefin metathesis product comprises one or a plurality of substructures having a formula —CH═CH—CH₂—CH═CH—.

3. The invention of claim 1 wherein the optionally functionalized olefin reactant is selected from the group consisting of a polyunsaturated fatty acid, a derivative of a polyunsaturated fatty acid, a natural oil, a low-molecular weight olefin, or a combination thereof.

4. The invention of claim 1 wherein the metathesis reaction comprises self-metathesis of the optionally functionalized olefin reactant.

5. The invention of claim 1 wherein the metathesis reaction comprises cross-metathesis between the optionally functionalized olefin reactant and an optionally functionalized olefin co-reactant.

6. The invention of claim 5 wherein the optionally functionalized olefin reactant comprises a natural oil, and wherein the optionally functionalized olefin co-reactant comprises a low-molecular weight olefin.

7. The invention of claim 1 wherein the dehydrogenation suppression agent suppresses isomerization and dehydrogenation of the olefin metathesis product.

8. The invention of claim 1 wherein the hydrogen transfer agent comprises a hydrogenation catalyst.

9. The invention of claim 1 wherein the dehydrogenation suppression agent comprises a hydrogen transfer inhibitor.

10. The invention of claim 1 wherein the dehydrogenation suppression agent comprises phosphorous.

11. The invention of claim 10 wherein the dehydrogenation suppression agent comprises a material selected from the group consisting of PH₃, a phosphine, a phosphonium salt, a phosphine oxide, a phosphorous oxo acid, a salt of a phosphorous oxo acid, an ester of a phosphorous oxo acid, a derivative of a phosphorous oxo acid in which at least one P—H bond has been replaced by a P—C bond, a salt of the derivative, an ester of the derivative, and combinations thereof.

12. The invention of claim 11 wherein the phosphine comprises a structure P(R¹)(R²)(R³), wherein R¹, R², and R³ are alike or different and are each independently selected from the group consisting of hydrogen, substituted or unsubstituted optionally functionalized C₁-C₁₀₀ alkyl, substituted or unsubstituted optionally functionalized aryl, and combinations thereof; wherein two of R¹, R², and R³ taken together may optionally form a ring with phosphorous; and wherein covalent bonds may optionally exist between two or more of R¹, R², and R³.

13. The invention of claim 11 wherein the phosphonium salt comprises a structure selected from the group consisting of [⁺(R¹)(R²)(R³)(R⁴)]X⁻, [⁺P(R¹)(R²)(R³)(R⁴)]₂X²⁻, and a combination thereof, wherein R¹, R², R³, and R⁴ are alike or different and are each independently selected from the group consisting of hydrogen, substituted or unsubstituted optionally functionalized C₁-C₁₀₀ alkyl, substituted or unsubstituted optionally functionalized aryl, and combinations thereof; wherein X represents an anion; wherein two of R¹, R², R³, and R⁴ taken together may optionally form a ring with phosphorous; and wherein covalent bonds may optionally exist between two or more of R¹, R², R³, and R⁴.

14. The invention of claim 11 wherein the ester of the derivative comprises a structure selected from the group consisting of R¹HP(O)OR², R³R⁴P(O)OR⁵, and a combination thereof; wherein R¹, R², R³, R⁴, and R⁵ are alike or different and are each independently selected from the group consisting of substituted or unsubstituted optionally functionalized C₁-C₁₀₀ alkyl, substituted or unsubstituted optionally functionalized aryl, and combinations thereof; wherein R¹ and OR² taken together may optionally form a bond with phosphorous; wherein a covalent bond may optionally exist between R¹ and R²; wherein two of R³, R⁴, and OR⁵ taken together may optionally form a ring with phosphorous; and wherein covalent bonds may optionally exist between two or more of R³, R⁴, and R⁵.

15. The invention of claim 10 wherein the dehydrogenation suppression agent is selected from the group consisting of phosphoric acid, phosphorous acid, phosphinic acid, phosphonic acid, phosphinic acid, phosphinous acid, and combinations thereof.

16. The invention of claim 10 wherein the dehydrogenation suppression agent comprises phosphorous acid, which is provided in an aqueous solution having a concentration of between about 0.1 wt % and about 70 wt %.

17. The invention of claim 10 wherein the dehydrogenation suppression agent comprises phosphinic acid, which is provided in an aqueous solution having a concentration of between about 0.1 wt % and about 50 wt %.

18. The invention of claim 10 wherein the dehydrogenation suppression agent comprises a phosphite ester having a structure P(OR¹)(OR²)(OR³), wherein R¹, R², and R³ are alike or different and are each independently selected from the group consisting of substituted or unsubstituted optionally functionalized C₁-C₁₀₀ alkyl, substituted or unsubstituted optionally functionalized aryl, and combinations thereof; wherein two of OR¹, OR², and OR³ taken together may optionally form a ring with phosphorous; and wherein covalent bonds may optionally exist between two or more of R¹, R², and R³.

19. The invention of claim 1 wherein the dehydrogenation suppression agent comprises nitrogen.

20. The invention of claim 19 wherein the dehydrogenation suppression agent is a material selected from the group consisting of ammonia, primary amines, secondary amines, tertiary amines, ammonium salts, polyamines, nitric acid, and combinations thereof.

21. The invention of claim 20 wherein the primary amines are selected from the group consisting of optionally functionalized alkyl amines, optionally functionalized aryl amines, and combinations thereof; wherein the secondary amines and tertiary amines are each independently selected from the group consisting of optionally functionalized alkyl amines, optionally functionalized aryl amines, optionally functionalized mixed alkyl-aryl amines, and combinations thereof; and wherein the ammonium salts comprise ammonium cations selected from the group consisting of optionally functionalized tetraalkylammoniums, optionally functionalized tetraarylammoniums, optionally functionalized mixed alkyl-aryl ammoniums, and combinations thereof.

22. The invention of claim 20 wherein the primary amines comprise a structure having a formula NH₂R, wherein R is selected from the group consisting of substituted or unsubstituted optionally functionalized C₁-C₁₀₀ alkyl, substituted or unsubstituted optionally functionalized aryl, and combinations thereof.

23. The invention of claim 20 wherein the secondary amines comprise a structure having a formula NHR¹R², wherein R¹ and R² are alike or different and are each independently selected from the group consisting of substituted or unsubstituted optionally functionalized C₁-C₁₀₀ alkyl, substituted or unsubstituted optionally functionalized aryl, and combinations thereof; wherein R¹ and R² taken together may optionally form a ring with nitrogen; and wherein covalent bonds may optionally exist between R¹ and R².

24. The invention of claim 20 wherein the tertiary amines comprise a structure having a formula NR¹R²R³, wherein R², and R³ are alike or different and are each independently selected from the group consisting of substituted or unsubstituted optionally functionalized C₁-C₁₀₀ alkyl, substituted or unsubstituted optionally functionalized aryl, and combinations thereof; wherein two of R¹, R², and R³ taken together may optionally form a ring with nitrogen; and wherein covalent bonds may optionally exist between two or more of R², and R³.

25. The invention of claim 20 wherein the ammonium salts comprise a structure selected from the group consisting of [⁺N(R¹)(R²)(R³)(R⁴)]X⁻, [⁺N(R¹)(R²)(R³)(R⁴)]₂X²⁻, and a combination thereof, wherein R¹, R², R³, and R⁴ are alike or different and are each independently selected from the group consisting of hydrogen, substituted or unsubstituted optionally functionalized C₁-C₁₀₀ alkyl, substituted or unsubstituted optionally functionalized aryl, and combinations thereof; wherein X represents an anion; wherein two of R¹, R², R³, and R⁴ taken together may optionally form a ring with nitrogen; and wherein covalent bonds may optionally exist between two or more of R¹, R², R³, and R⁴.

26. The invention of claim 20 wherein the polyamines comprise a structure R⁶R⁷N-L-NR⁸R⁹, wherein R⁶, R⁷, R⁸, and R⁹ are alike or different and are each independently selected from the group consisting of hydrogen, substituted or unsubstituted optionally functionalized C₁-C₁₀₀ alkyl, substituted or unsubstituted optionally functionalized aryl, and combinations thereof; and wherein L is a linker selected from the group consisting of (i) substituted or unsubstituted, optionally functionalized aryl groups, (ii) cyclic or acyclic, substituted or unsubstituted, optionally functionalized alkyl groups, and (iii) combinations thereof.

27. The invention of claim 20 wherein the nitric acid is selected from the group consisting of anhydrous nitric acid, fuming nitric acid, concentrated nitric acid, solid hydrates of nitric acid, solutions of nitric acid, and combinations thereof.

28. The invention of claim 1 wherein the dehydrogenation suppression agent comprises a quinone, a hydroquinone, or a combination thereof.

29. The invention of claim 28 wherein the quinone is an electron-deficient quinone.

30. The invention of claim 28 wherein the quinone is selected from the group consisting of optionally functionalized benzoquinones, optionally functionalized naphthoquinones, optionally functionalized anthraquinones, and combinations thereof.

31. The invention of claim 28 wherein the quinone is selected from the group consisting of 1,2-benzoquinone, 1,4-benzoquinone, tetrachloro-p-benzoquinone, 2-chloro-1,4-benzoquinone, 2,6-dichloro-1,4-benzoquinone, difluoro-1,4-benzoquinone, trifluoro-1,4-benzoquinone, tetrafluoro-1,4-benzoquinone, 2,5-dichlorobenzoquinone, 2,3-dichloro-5,6-dicyano-1,4-benzoquinone, 1,2-naphthoquinone, 1,4-naphthoquinone, 2,6-naphthoquinone, 9,10-anthraquinone, 2-hydroxy-1,4-naphthoquinone, 2-chloro-1,4-naphthoquinone, 2,3-dichloro-1,4-naphthoquinone, 2-bromo-1,4-naphthoquinone, 2,3-dibromo-1,4-naphthoquinone, plastoquinone, phylloquinone, ubiquinone, 2,3-dihydroxy-9,10-anthraquinone, 2,6-dichloro-1,4-benzoquinone, tetrachloro-1,4-benzoquinone, 2,6-dimethoxy-1,4-benzoquinone, 2,6-di-tert-butyl-1,4-benzoquinone, and combinations thereof.
